(19) United States
(12) Patent Application Publication
(10) Pub. No.: US 2023/0183766 A1
(43) Pub. Date: Jun. 15, 2023

(54) BACTERIAL STRAINS AND METHOD FOR PRODUCING OLIGOSACCHARIDES

(71) Applicants: Institut National des Sciences Appliquées de Toulouse, Toulouse (FR); Institut national de recherche pour l’agriculture, l’alimentation et l’environnement, Paris (FR); Centre National de la Recherche Scientifique, Paris (FR); Université Toulouse III - Paul Sabatier, Toulouse (FR)

(72) Inventors: Julien Durand, Toulouse (FR); Fabien Letisse, Toulouse (FR); Pietro Tedesco, Toulouse (FR); Gabrielle Veronese, Toulouse (FR)

(21) Appl. No.: 17/924,593

(22) PCT Filed: May 11, 2021
(86) PCT No.: PCT/FR2021/050827

§ 371 Date: Nov. 10, 2022

(30) Foreign Application Priority Data
May 12, 2020 (FR) FR2004669

(51) Int. Cl. C12P 19/12; C12N 15/52; C12N 15/70; C12N 9/12; C12N 1/20

(52) U.S. Cl. CPC C12P 19/12; C12N 15/52; C12N 15/70; C12N 9/12; C12N 1/20; C12N 2500/34

(57) ABSTRACT

The present invention relates to a strain deposited with the CNCM (Collection Nationale de Culture de Microorganismes, Institut Pasteur, 25 rue du Docteur Roux, 75724 Paris Cedex 15, France), under number CNCM I-5499. The present invention also relates to the in vitro use of strains for producing oligosaccharides and/or in a process for producing oligosaccharides.

The present invention finds an application, in particular in the bioproduction field, for example the production of compounds, for example of bio-compounds.

### TECHNICAL FIELD

The present invention relates to bacterial strains and also to the in vitro use of bacterial strains for the production of oligosaccharides and/or in a process for producing oligosaccharides.

The present invention also relates to an in vitro, in particular in cellulo, process for producing oligosaccharides.

The present invention finds an application, especially in the bioproduction field, for example the production of compounds, for example of bio-compounds.

In the following description, references enclosed in brackets ([ ]) refer to the list of references presented at the end of the text.

### STATE OF THE ART

Oligosaccharides represent a very diverse class of biomolecules made of linear or branched chains of glycosylated units linked together by different types of a or β-osidic bonds. Their structural diversity results from the nature of the monomers, from the position and from the anomerism of their covalent bonds (α or β) at the same time. These molecules play diverse and important roles in a multitude of cellular processes, including cell signaling and differentiation, modulation of the immune response and of the inflammation, mediation of microbial interactions, and host-microbe interactions. Due to their very diversified structures and biological functions, oligosaccharides are used in a wide range of applications in the food industry (functional foods, prebiotics or additives) and in the health industry (as basic elements for the chemical synthesis of drugs, screening kits, pharmaceutical carriers or vaccine intermediates).

Therefore, the interest in the production of oligosaccharides is increasing very rapidly. The challenge is to develop synthesis routes in accordance with the principles of green chemistry (Anastas and Warner, 2000 Anastas, P T, Warner, J C, 2000. Green chemistry: theory and practice, 1. paperback. ed. Oxford Univ. Press, Oxford. [15]), based on the use of inexpensive and versatile bioresources, to produce a wide diversity of oligosaccharides.

There is therefore a real need to find a means and/or process allowing to control the production of oligosaccharides while reducing the production costs of various oligosaccharides and/or to improve the production yields, while avoiding and/or reducing and/or eliminating the use of compounds and/or solutions environmentally harmful.

There are currently different processes for the production of oligosaccharides.

Some oligosaccharides are currently produced by chemical synthesis. This approach requires multiple reaction and purification steps to protect and deprotect the functional groups and to perform regio- and stereospecific syntheses. Despite significant progress in this field (Rudroff, F., Mihovilovic, M. D., Gröger, H., Snajdrova, R., Iding, H., Bornscheuer, U. T., 2018. Opportunities and challenges for combining chemo- and biocatalysis. Nature Catalysis 1, 12-22. https://doi.org/10.1038/s41929-017-0010-4 [18]), these production routes, in most cases, lead to a low level of production (Ager, 2012 Ager, D., 2012. Final Analysis: Biological or Chemical Catalysis Platinum Metals Review 56 [19]), and require the use of polluting solvents. In addition, the diversity of the oligosaccharide structures which can be obtained by chemical synthesis is limited.

Also, there is therefore a real need to find a means and/or process allowing to control the production of oligosaccharides while reducing the costs, especially of production, to improve the production yields while avoiding and/or reducing and/or eliminating the use of compounds and/or solutions environmentally harmful.

Chemoenzymatic and enzymatic oligosaccharide production routes have been developed over the past twenty years in order to take advantage of the natural diversity and selectivity of enzymes, and thus broaden the panel of oligosaccharide structures that can be produced by biocatalysis (Combes, Monsan, 2009. Biocatalyse ou catalyse enzymatique. Procédés chimie-bio-agro I Chimie verte, Techniques de l'ingénieur 1-18. [13]; Reetz, M. T., 2013. Biocatalysis in Organic Chemistry and Biotechnology: Past, Present, and Future. Journal of the American Chemical Society 135, 12480-12496. https://doi.org/10.1021/ja405051f [20]; Rudroff, F., Mihovilovic, M. D., Gröger, H., Snajdrova, R., Iding, H., Bornscheuer, U. T., 2018. Opportunities and challenges for combining chemo- and biocatalysis. Nature Catalysis 1, 12-22. [14]). However, the use of enzymes in in vitro processes is limited due to the enzyme production and purification cost, the loss of activity of enzymes during their storage, and their implementation, and furthermore the cost of the substrates/raw materials required.

There is therefore a real need to find a means and/or process and/or new processes allowing to control the production of oligosaccharides on a large scale, having economically viable production costs and with a low environmental impact.

Five main types of enzymes have been described as being able to be used to effectively catalyze the production of oligosaccharides: glycoside-hydrolases (GFI), glycosynthases (GS), transglycosylases (TG), glycoside-transferases (GT) and glycoside-phosphorylases (GP). However, each presents drawbacks and limitations.

GH allow to hydrolyze more or less specifically the glycans to obtain mixtures of oligosaccharides. Their use is nevertheless limited to the conversion of very abundant polymers, such as those resulting from plant biomass, and, in the majority of cases, their product specificity is not sufficient to give fractions of monodispersed oligosaccharides.

GS are GH mutants produced by enzymatic engineering. They require expensive synthetic substrates.

TG can perform the synthesis using inexpensive substrates (such as sucrose or starch) but their natural structural and functional diversity is limited to the transfer of a very limited type of glycosyl units.

GT allow the synthesis of oligosaccharides from very expensive activated substrates (glycosyl-mono- or diphosphonucleotides, and glycosyl-polyprenol mono- or diphosphates), which makes their in vitro use not very profitable.

GP can catalyze oligosaccharides synthesis reactions during the “reverse phosphorolysis” reaction

The so-called “reverse phosphorolysis” reaction (Puchart, V., 2015. Glycoside phosphorylases: Structure, catalytic properties and biotechnological potential. Biotechnology Advances 33, 261-276. https://doi.org/10.1016/j.biotechadv.2015.02.002 [21]), forming an osidic bond between the glycosidic unit derived from a glycosyl-phosphate, which acts as a glycosyl “donor”, and a hydroxylated “acceptor”, is shown in the diagram below. However, without continuous removal of the reverse phosphorolysis product or the released inorganic phosphate to alter the reaction equilibrium, the synthesis yields of oligosaccharides are low and the high cost of glycosyl-phosphates remains a significant hindrance to the use of these enzymes in industrial in vitro processes.

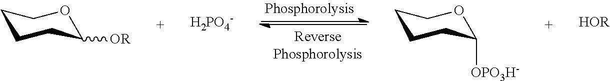

There is therefore a real need to find a means and/or process allowing to control the production of oligosaccharides, in particular oligosaccharides on a large scale, having economically viable production costs and with a low environmental impact.

In vitro synthetic processes based on the use of GS have been described (Cobucci-Ponzano, B., Moracci, M., 2012. Glycosynthases as tools for the production of glycan analogs of natural products. Natural Product Reports 29, 697. [22]). They require chemically modified glycosyl donors which are difficult to produce in high yield at reasonable cost. GT are used for the production of oligosaccharides, in particular using recombinant strains of Escherichia coli (Samain & Priem (2001) Procédé de production d'oligosaccharides WO0104341 [23]; WO2008040717 Samain (2008) High yield production of sialic acid (Neu5ac) by fermentation [24]; WO2007101862 Samain (2007) Method of producing sialylated oligosaccharides [54]; WO2007023348, SAMAIN et al. (2007) Production of globoside oligosaccharides using metabolically engineered microorganisms [55]; Fort, S., Birikaki, L., Dubois, M. P., Antoine, T., Samain, E., & Driguez, H. (2005). Biosynthesis of conjugatable saccharidic moieties of GM 2 and GM 3 gangliosides by engineered E. coli. Chemical communications, (20), 2558-2560 [56]; Fierfort, N., & Samain, E. (2008). Genetic engineering of Escherichia coli for the economical production of sialylated oligosaccharides. Journal of biotechnology, 134β-4), 261-265) [57].

Some of these known processes use GP in recombinant strains in the direction of the phosphorolysis, to increase the intracellular concentration of phosphorylated monosaccharides.

There is therefore a real need to find a means and/or product and/or process that is simple and/or does not comprise steps that are complex and/or can only be implemented by an expert, for the production of oligosaccharides especially in cellulo via a suitable microorganism.

There is therefore a real need to find a means and/or product and/or process allowing to control the production of oligosaccharides, in particular the type of produced oligosaccharides, and also to reduce the costs, especially of production, while improving the yields and amount of produced oligosaccharides.

### DESCRIPTION OF THE INVENTION

The present invention makes it possible to resolve and overcome the obstacles and drawbacks of the aforementioned prior art by providing a strain filed on Feb. 26, 2020 with the CNCM (Collection Nationale de Culture de Microorganismes, Institut Pasteur, 25 rue du Docteur Roux, 75724 Paris Cedex 15, France), under number CNCM I-5499.

The present invention makes it possible to resolve and overcome the obstacles and drawbacks of the aforementioned prior art by providing strain filed with the CNCM (Collection Nationale de Culture de Microorganismes, Institut Pasteur, 25 rue du Docteur Roux, 75724 Paris Cedex 15, France), under numbers CNCM I-5499, CNCM I-5681 and CNCM I-5682.

In particular, the inventors have advantageously developed a new means of production of oligosaccharides. In particular, the strain according to the invention allows the production of oligosaccharides via glycoside-phosphorylases and also advantageously both an internalization of precursors for the synthesis, in particular of non-phosphorylated carbohydrates, and an intracellular accumulation of phosphorylated carbohydrates.

In particular, the inventors have surprisingly and unexpectedly demonstrated that the strain according to the invention advantageously allows to accumulate especially non-phosphorylated carbohydrates required for the synthesis of oligosaccharides by the glycoside-phosphorylases.

The inventors have also surprisingly demonstrated that the strain according to the invention advantageously allows, during the production of oligosaccharides, the excretion of said oligosaccharides in the culture medium. Advantageously, the inventors have demonstrated that the strain according to the invention allows recovery and facilitated purification of the produced oligosaccharides due to their excretion in the culture medium.

In addition, advantageously the diffusion or excretion of the oligosaccharides in the extracellular medium could allow to obtain a reaction catalyzed by the glycoside-phosphorylases favored in the direction of reverse phosphorolysis.

The present invention advantageously allows, especially with respect to the chemical syntheses of known oligosaccharides, a production of oligosaccharides which enormously reduces the environmental impact.

The inventors have thus demonstrated that the invention advantageously allows to provide a new process and/or a new route for the synthesis of oligosaccharides which overcomes the drawbacks encountered in the known processes for the synthesis of oligosaccharides, especially in in vitro enzymatic syntheses without using a living biological support.

The present invention allows as mentioned above and in a synergistic manner to considerably reduce the production costs, the number of synthesis and purification steps with respect to the synthetic processes, especially in vitro chemical and enzymatic synthetic processes, without using a living biological support.

The present invention relates to an Escherichia coli strain whose recA1, gyrA96, thi-1, glnV44 relA1 hsdR17, endA1, lacZ, nanKETA, lacA, melA, wcaJ, mdoH, ptsG, manX, manY, and pfkA genes are inactivated.

According to the invention, it may be, for example, the strain filed with the CNCM (Collection Nationale de Culture de Microorganismes, Institut Pasteur, 25 rue du Docteur Roux, 75724 Paris Cedex 15, France), under number CNCM I-5499 which is a strain derived from Escherichia coli in that the recA1, gyrA96, thi-1, glnV44 relA1 hsdR17, endA1, lacZ, nanKETA, lacA, melA, wcaJ, mdoH, ptsG, manX, manY, and pfkA genes are inactivated

According to the invention, the strain filed with the CNCM (Collection Nationale de Culture de Microorganismes, Institut Pasteur, 25 rue du Docteur Roux, 75724 Paris Cedex 15, France), under number CNCM I-5499 is a strain derived from Escherichia coli whose recA1, gyrA96, thi-1, glnV44 relA1 hsdR17, endA1, lacZ, nanKETA, lacA, melA, wcaJ, mdoH, ptsG, manX, manY, and pfkA genes are inactivated

The inventors have also surprisingly demonstrated that the mutation, namely the additional inactivation of at least one of the maa and manA genes, preferably the inactivation of the maa and manA genes advantageously allows an increase in the production yield of oligosaccharides and to avoid the production of any parasitic product and/or considered to be a contaminant, for example such as acetylated sugars.

Also, the strain according to the invention may further comprise the Δmaa and ΔmanA mutations.

In the present, by Δmaa it is meant the inactivation of the maa gene.

In the present, by ΔmanA it is meant the inactivation of the manA gene.

In the present, the gene inactivation can be performed by any suitable process known to the person skilled in the art. This may involve, for example, the process described in Kirill A. Datsenko, Barry L. Wanner. One-step inactivation of chromosomal genes in Escherichia coli K-12 using PCR products. Proceedings of the National Academy of Sciences June 2000, 97 (12) 6640-6645; DOI: 10.1073/pnas.120163297 [62].

The present invention also relates to the strain filed on May 7, 2021 with the CNCM (Collection Nationale de Culture de Microorganismes, Institut Pasteur, 25 rue du Docteur Roux, 75724 Paris Cedex 15, France), under number CNCM I-5681 which is an Escherichia coli strain whose recA1, gyrA96, thi-1, glnV44 relA1 hsdR17, endA1, lacZ, nanKETA, lacA, melA, wcaJ, mdoH, ptsG, manX, manY, pfkA, maa and manA genes are inactivated.

In the present invention, by recA1 it is meant the gene encoding the RecA 1 protein (NCBI Gene ID: 947170 UniProtKB-P0A7G6)

Herein by gyrA96 it is meant the gene encoding the A subunit of the DNA gyrase (NCBI GeneID:946614 UniProtKB-P0AES4)

In the present invention, by thi-1 it is meant the gene encoding the Thiamine thiazole synthase, chloroplastic (NCBI GeneID:948491 UniProtKB P30137)

In the present invention, by glnV44 it is meant the gene encoding a glutamine tRNA (NCBI GeneID:945255).

In the present invention, by relA1 it is meant the gene encoding the GTP pyrophosphokinase (NCBI GeneID:947244 UniProtKB-P0AG20).

In the present invention, by hsdR17 it is meant a mutant of the hsdR gene encoding an endonuclease component of the type I EcoR12411 R protein restriction enzyme (NCBI GeneID: 948878 UniProtKB-P10486)

In the present invention, by endA1 it is meant the gene encoding the EndA1 endonuclease (NCBI GeneID:949092 UniProtKB-P25736)

In the present invention, by lacZ it is meant the gene encoding for the β-galactosidase (NCBI GeneID: 945006 UniProtKB-P00722)

In the present invention, by nanKETA it is meant the genes encoding the operon allowing an internalization and the metabolization of sialic acid (Neu5Ac), in particular genes: nanA encoding an N-acetylneuraminate lyase (NCBI GeneID:947742 UniProtKB-P0A6L4); nanT encoding the N-acetylneuraminate:H+ symporter (NCBI GeneID:947740; UniProtKB-P41036); nanE encoding the N-acetylmannosamine-6-phosphate epimerase (NCBI GeneID:947745 UniProtKB-P0A761); nanK encoding the N-acetylmannosamine kinase (NCBI GeneID:947757 UniProtKB-P45425).

In the present invention, by lacA it is meant the gene encoding for the galactoside O-acetyltransferase (NCBI GeneID: 945674 UniProtKB-P07464).

In the present invention, by melA it is meant the gene encoding the α-galactosidase (NCBI eneID:948636 UniProtKB-P06720).

In the present invention, by wcaJ it is meant the gene encoding the UDP-glucose:undecaprenyl-phosphate glucose-1-phosphate transferase (NCBI GeneID:946583 UniProtKB-P71241).

In the present invention, by mdoH it is meant the gene encoding the osmoregulated periplasmic glucans (OPGs) biosynthesis protein H (NCBI GeneID:945624 UniProtKB-P62517).

In the present invention, by ptsG it is meant the gene encoding the glucose-specific PTS enzyme IIBC component (NCBI GeneID: 945651 UniProtKB-P69786).

In the present invention, by manX it is meant the gene encoding the mannose-specific PTS enzyme IIAB component (NCBI GeneID: 946334 UniProtKB-P69797).

In the present invention, by manY it is meant the gene encoding the mannose-specific PTS enzyme IIC component (NCBI GeneID: 946332 UniProtKB-P69801)

In the present invention, by pfkA it is meant the gene encoding for the ATP-dependent 6-phosphofructokinase isozyme 1 (NCBI GeneID: 948412 UniProtKB-P0A796).

In the present invention, by maa it is meant the gene encoding for the Maltose 0-acetyltrasferase (NCBI GeneID: 414992 UniProtKB-P77791);

In the present invention, by manA it is meant the gene encoding for the Mannose-6-phosphate isomerase (NCBI GeneID: 416130 UniProtKB-P00946);

According to the invention, the strain can further be genetically modified, by any suitable process known to the person skilled in the art. It may be, for example, at least one mutation, deletion and/or substitution.

According to the invention, the strain can for example be genetically modified. It may be, for example, a genetic modification of the expression of a gene. For example the strain may comprise the substitution of a promoter. For example, the strain may comprise the substitution of the promoter of the galP gene by a constitutive promoter. It may be, for example, any suitable constitutive promoter known to the person skilled in the art. It may be, for example, the promoter of the gene encoding IHF, also referred to as HIF promoter, for example as described in Zhou, K., Zhou, L., Lim, Q. 'En, Zou, R., Stephanopoulos, G., and Too, H.-P. (2011) Novel reference genes for quantifying transcriptional responses of Escherichia coli to protein overexpression by quantitative PCR. BMC Mol Biol 12: 18 [65]. It may be, for example, the promoter of the gene encoding IHF (“Integration host factor”) NCBI Grendel 415432, UniProtKB-P0A6Y1). It may be, for example, the IHF promoter of sequence

(SEQ ID NO: 66)
TCTGAAACAGTGCAAAGCTAACCCGTGGCAGCAGTTCGCGGAAACCCAC
AACAAGGGCGACCGTGTTGAAGGTAAAATCAAGTCTATCACTGACTTCG
GTATCTTCATCGGCTTGGACGGCGGCATCGACGGCCTGGTTCACCTGTC
TGACATCTCCTGGAACGTTGCAGGCGAAGAAGCAGTTCGTGAATACAAA
AAAGGCGACGAAATCGCTGCAGTTGTTCTGCAGGTTGACGCAGAACGTG
AACGTATCTCCCTGGGCGTTAAACAGCTCGCAGAAGATCCGTTCAACAA
CTGGGTTGCTCTGAACAAGAAAGGCGCTATCGTAACCGGTAAAGTAACT
GCAGTTGACGCTAAAGGCGCAACCGTAGAACTGGCTGACGGCGTTGAAG
GTTACCTGCGTGCTTCTGAAGCATCCCGTGACCGCGTTGAAGACGCTAC
CCTGGTTCTGAGCGTTGGCGACGAAGTTGAAGCTAAATTCACCGGCGTT
GATCGTAAAAACCGCGCAATCAGCCTGTCTGTTCGTGCGAAAGACGAAG
CTGACGAGAAAGATGCAATCGCAACTGTTAACAAACAGGAAGATGCAAA
CTTCTCCAACAACGCAATGGCTGAAGCTTTCAAAGCAGCTAAAGGCGAG
TAATTCTCTGACTCTTCGGGATTTTTATTCCGAAGTTTGTTGAGTTTAC
TTGACAGATTGCAGGTTTCGTCCTGTAATCAAGCACTAAGGGCGGCTAC
GGCCGCCCTTAATCAATGCAGCAACAGCAGCCGCTTAATTTGCCTTTAA
GGAACCGGAGGAATC.

Advantageously, when the strain comprises a constitutive promoter for the galP gene, the inventors have demonstrated a possible increase in the production yield of oligosaccharides.

In the present, the substitution of a DNA sequence can be carried out by any suitable process known to the person skilled in the art. It may be, for example, the Datsenko and Wanner protocol (Kirill A. Datsenko, Barry L. Wanner. One-step inactivation of chromosomal genes in Escherichia coli K-12 using PCR products. Proceedings of the National Academy of Sciences June 2000, 97 (12) 6640-6645; DOI: 10.1073/pnas. 120163297 [62]).

The present invention also relates to the strain filed on May 7, 2021 with the CNCM (Collection Nationale de Culture de Microorganismes, Institut Pasteur, 25 rue du Docteur Roux, 75724 Paris Cedex 15, France), under number CNCM I-5682 which is an Escherichia coli strain whose recA1, gyrA96, thi-1, glnV44 relA1 hsdR17, endA1, lacZ, nanKETA, lacA, melA, wcaJ, mdoH, ptsG, manX, manY, pfkA, maa and manA genes are inactivated, and comprising a constitutive promoter, preferably the IHF promoter of sequence SEQ ID NO: 66, for the galP gene.

According to the invention, the strain may comprise at least one expression vector.

In the present invention, by expression vector it is meant any suitable expression vector known to a person skilled in the art, especially, for the expression of genes encoding proteins in microorganisms. It may be any suitable expression vector known to the person skilled in the art for the expression of genes encoding proteins in a bacterium. It may be, for example, any vector selected from the vectors listed in the catalog https://france.promega.com/products/vectors/protein-expression-vectors [35] or in the catalog https://www.thermofisher.com/search/browse/category/fr/fr/85801/Bacterial %20Expression %20 Vectors [36]. It may be, for example, the expression vector described in document WO 83/004261 [37]. The vector can be, for example a plasmid, a yeast artificial chromosome (YAC) or a bacterial artificial chromosome (BAC). It may be, for example, any suitable plasmid known to the person skilled in the art for the expression of genes encoding proteins in bacteria and/or commercially available. It may be, for example, the pWKS130 plasmid described in the document Rong Fu Wang, Kushner, S R, 1991. Construction of versatile low-copy-number vectors for cloning, sequencing and gene expression in Escherichia coli. Gene 100, 195-199. [10], the plasmid commercialized by the Thermofisher company under the commercial reference pBAD-HisA, the plasmid commercialized by the Thermofisher company under the commercial reference pTRC. It may be, for example, a plasmid selected from the group comprising pBAD-HisA, pTRC, pMX, pUC19, pHP45-CmR, pcDNA3.1(+), pcDNA3.3-TOPO, pcDNA3.4-TOPO, pFastBac1, pET100/D-TOPO, pET151/D-TOPO, pRSETA, pYes2.1V5-His TOPO, pDONR221, pGEX-3X; pBluescript, pET, pGEX, pLys, pRARE, pDEST, pETG, preferably the pBAD-HisA plasmid. It may be, for example, any expression vector mentioned, for example in the internet pages https://www.embl.de/pepcore/pepcore_services/strains_vectors/vectors/bacterial_expression_vec tors/popup_bacterial_expression_vectors/index.html [38], http://babel.ucmp.umu.se/cpep/web_content/Pages/CPEP_09_vectors.html [39] and/or https://www.embl.de/pepcore/pepcore_services/strains_vectors/vectors/gateway_vectors/index.ht m [40]

The vector can be any suitable yeast artificial chromosome (YAC) for expression in bacteria, known to the person skilled in the art and/or commercially available. It may be, for example, a yeast artificial chromosome selected from the group comprising pYAC-RC, pYAC3.

The vector can be any suitable bacterial artificial chromosome (BAC) for expression in bacteria, known to the person skilled in the art and/or commercially available. It may be, for example, a bacterial artificial chromosome selected from the group comprising pUvBBAC, pCC1BAC, pBAC 108L.

According to the invention, at least one expression vector can allow the expression of at least one glycoside-phosphorylase. For example, the expression vector can allow the expression of a glycoside-phosphorylase selected from β-glycoside- or α-glycoside-phosphorylases, for examples α-1,3-glucopyranosyl-L-rhamnose-phosphorylases, α-1,2-glucosyl-glycerol-phosphorylases, trehalose-phosphorylases, laminaribiose-phosphorylases, D-galactosyl-β-1,4-L-rhamnose phosphorylases, β-1,4-mannosyl-glucose-phosphorylases, β-1,2-oligomannan-phosphorylases, β-1,2-mannobiose-phosphorylases, β-1,4-mannopyranosyl-[N-glycane]-phosphorylases/β-1,4-mannopyranosyl-chitobiose-phosphorylases, β-1,4-mannooligosaccharide-phosphorylases, β-1,3-mannooligosaccharide-phosphorylases, β-1,3-mannosyl-glucose phosphorylases, β-1,4-mannosyl-glucuronate phosphorylases.

For example, the expression vector can allow the expression of a glycoside phosphorylase as listed in Table 1 below.

Table 1 describes examples of glycoside-phosphorylases and mentions their GenBank or Gigabase identifier.

TABLE 1
GLYCOSIDE-PHOSPHORYLASES AND GENBANK
OR GIGABASE ACCESSION NUMBERS

| Glycoside-phosphorylase | GenBank |
|---|---|
| 3-O-α-glucopyranosyl-L-rhamnose phosphorylase | ABX41399.1 |
| (Cphy_1019) | |
| 1,2-alpha-glucosylglycerol phosphorylase (Bsel_2816) | ADI00307.1 |
| trehalose phosphorylase (TreP) | BAB97299.1 |
| trehalose phosphorylase (TPase) | BAC20640.1 |
| D-galactosyl-1,4-L-rhamnose phosphorylase | ABX42289.1 |
| (Cphy_1920) | |
| D-galactosyl-β-1,4-L-rhamnose phosphorylase | ACB74662.1 |
| (GalRhaP;Oter_1377) | |
| β-1,4-mannosylglucose phosphorylase (Unk1) | AAS 19693.1 |
| β-1,2-oligomannan phosphorylase (Teth514_1788) | ABY93073.1 |
| β-1,2-mannobiose phosphorylase (Teth514_1789) | ABY93074.1 |
| β-1,4-mannopyranosyl-[N-glycane] phosphorylase/β- | ADD61463.1 |
| 1,4-mannopyranosyl-chitobiose phosphorylase | |
| (Uhgb_MP) | |
| β-1,4-mannooligosaccharide phosphorylase | ADU20661.1 |
| (MOP;RaMP2;Rumal_0099) | |
| β-1,4-mannosylglucose phosphorylase | ADU21379.1 |
| (RaMPl;RaMGP;Rumal_0852) | |
| β-1,2-mannobiose phosphorylase (Lin0857) | CAC96089.1 |
| β-1,4-mannosylglucose phosphorylase (MGP;BF0772) | CAH06518.1 |
| β-1,3-mannooligosaccharide phosphorylase | CAZ94304.1 |
| (zobellia_231) | |
| CalpoDRAFT_0075 | WP_026485574.1 |
| CalpoDRAFT_1209 | WP_026486530.1 |
| Trehalose phosphorylase | AAF22230.1 |
| Trehalose phosphorylase | ABC84380.1 |
| Trehalose phosphorylase | BAA31350.1 |
| β-1,4-mannooligosaccharide phosphorylase | VCV21229.1 |
| (RIL182_01100;ROSINTL182_05474) | |
| mannosylglucose phosphorylase | VCV21228.1 |
| (RIL182_01099;ROSINTL182_07685) | |
| β-1,2-oligomannan phosphorylase MTP3 | XP_003872966.1 |
| (LMXM_10_1250) | CBZ24448.1 |
| β-1,2-oligomannan phosphorylase MTP4 | CBZ24449.1 |
| (LMXM_10_1260) | XP_003 872967.1 |
| β-1,2-oligomannan phosphorylase MTP6 | CBZ24451.1 |
| (LMXM_10_1280) | XP_003872969.1 |
| β-1,2-oligomannan phosphorylase MTP7 | CBZ24452.1 |
| (LMXM_10_1290) | XP_003872970.1 |
| laminaribiose phosphorylase (ACL_0729) | ABX81345.1 |
| laminaribiose phosphorylase (LbpA) | BAJ10826.1 |
| Glycoside-phosphorylase | GigaDB, DOI: |
| | 10.5524/100064 |
| β-1,4-mannosyl-glucuronate phosphorylase (“β-1,4- | MH0431_GL0150624 |
| mannosyl-glucuronic acid phosphorylase”) | |
| β-1,4-mannooligosaccharide phosphorylase | MH0373_GL0093988 |
| β-1,3-mannosyl-glucose phosphorylase β-1,3- | 340101.Vvad_PD3074 |
| mannooligosaccharide phosphorylase | |

An expression vector may comprise a polynucleotide sequence, for example a genetic cassette (“expression cassette”), comprising the following elements in the order 5′ to 3′: a promoter sequence; a nucleic acid sequence encoding an enzyme, preferably a glycoside phosphorylase.

In the present document, “polynucleotide(s)”, “oligonucleotide(s)”, “nucleic acid(s)”, “nucleotide(s)”, “polynucleic acid(s)” or any used grammatical equivalent refers to a polymeric form of nucleotides or nucleic acids of any length, either ribonucleotides or deoxyribonucleotides. This term refers to the primary structure of the molecule. Thus, this term comprises double and single stranded DNA, triplex DNA, as well as double and single stranded RNA. It also comprises the modified forms, for example by methylation and/or capping, and the unmodified forms of the polynucleotide. The term is also intended to comprise molecules which comprise unnatural or synthetic nucleotides as well as nucleotide analogs. The nucleic acid sequences and vectors disclosed or contemplated herein can be introduced into a cell, for example by transfection, transformation or transduction.

The glycoside-phosphorylase can be a glycoside-phosphorylase as defined above.

The promoter can be any suitable promoter known to the person skilled in the art for expression in microorganisms, preferably in bacteria. It may be, for example, a constitutive promoter or an inducible promoter. It may be, for example, a constitutive promoter selected from the group comprising the CMV, EF1A, or SV40 promoter. It may be, for example, a constitutive promoter mentioned on the internet page http://parts.igem.org/Promoters/Catalog/Constitutive [41], of any promoter of the E. coli K-12 strain known to the person skilled in the art, for example a promoter selected from the 3908 promoters of the E. coli K-12 strain listed on the biocyc.org site: https://biocyc.org/group?id=:ALL-PROMOTERS&orgid=ECOLI [42]. It may be, for example, an inducible promoter, for example pBAD or pLac. It may be, for example, an inducible promoter described and/or mentioned on the page: http://parts.igem.org/Promoters/Catalog [43].

The person skilled in the art, from its general knowledge, will know how to choose the promoter based on the nucleic sequence to be expressed and/or the host cell, preferably the strain according to the invention, for example the one filed with the CNCM (Collection Nationale de Culture de Microorganismes, Institut Pasteur, 25 rue du Docteur Roux, 75724 Paris Cedex 15, France), under number CNCM I-5499, or under number CNCM I-5681, or under number CNCM I-5682.

In the present invention, the insertion of the coding nucleic sequence can be carried out by any suitable process known to the person skilled in the art. it may be, for example, any method for cloning genes known to the person skilled in the art. It may be, for example, a process described and/or available on the internet page https://www.addgene.org/mol-bio-reference/cloning [31]. The person skilled in the art, from its general knowledge, will know how to adapt the processes and/or methods of insertion and/or cloning according to the vector and/or the gene.

In the present invention, the strain may further comprise at least one expression vector allowing the expression of at least one transport system, preferably of monosaccharides or of oligosaccharides.

According to the invention, the expression vector may be as defined above.

In the present invention, by transport system it is meant any suitable protein and/or set of transport proteins and/or protein transporter to transport monosaccharides known to the person skilled in the art. It may be for example a transport protein or permease of non-phosphorylated carbohydrates, preferably of monosaccharides, selected from the families of transporters comprising the “phosphotransferase system” (PTS). It may be, for example, a non-PTS transporter selected from the transporters of the “Major Facilitor Superfamily” (MFS) family, of the sodium-glucose transporter (SGLT) family, of the dependent Ton-B family and/or of the super family of “ATP-Binding Cassette” (ABC) transporters.

It may be, for example, a transporter of the “Major Facilitor Superfamily” (MFS) family selected from the group comprising Galactose permease (GalP), for example the Galactose permease (GalP) from E. coli or Salmonella sp, the Glucose permease (GIcP), for example the Glucose permease (GIcP) from Streptomyces coelicolor or the glucose permease (Gif) of Zymomonas mobilis.

It may be, for example, a transporter of the sodium-glucose transporter (SGLT) family selected from the group comprising the SgIS transporter, for example the SgIS transporter of the Vibrio parahemoliticus strain.

It may be, for example, a transporter of the superfamily of “ATP-Binding Cassette” (ABC) transporters selected from the group comprising the MglABC transporter, for example the MglABC transporter of E. coli, the malEFG transporter, for example the malEFG transporter of Thermos termophilus, the Teth514_1792 transporter, for example the Teth514_1792 transporter of Thermoanaerobacter sp, or the Teth514_1796 transporter, for example the Teth514_1796 transporter of Thermoanaerobacter sp X-514.

In the present invention, an expression vector may allow the production of at least one transport system as mentioned above.

For example, the expression vector may allow the expression of a permease or transport protein as mentioned in Table 2 below.

Table 2 describes examples of a permease or transport protein and mentions their family.

TABLE 2
PERMEASE OR TRANSPORT PROTEIN AND
CORRESPONDING TRANSPORTER FAMILY

| Permease or transport protein | Family |
|---|---|
| Galactose permease (GalP) E. coli K12 | MFS |
| Galactose permease (GalP) Salmonella sp | MFS |
| Glucose permease (GlcP) Streptomyces coelicolor | MFS |
| Glucose facilitated factor permease (glf) Z. mobilis | MFS |
| Sodium-glucose transporter SglS of Vibrio parahemoliticus | SGLT |
| MglABC of E. coli | ABC |
| malEFG of Thermos termophilus | ABC |
| Teth514_1792 | ABC |
| Teth514_1796 | ABC |

In the present invention, an expression vector allowing the expression of at least one gene encoding a transport protein or permease may comprise a polynucleotide sequence, for example a genetic cassette (“expression cassette”), comprising the following elements in the order 5′ to 3′:

a promoter sequence;

a nucleic acid sequence encoding a protein, preferably a transport protein or permease.

The transport protein or permease can be a transport protein or permease as defined above.

According to the invention, the promoter may be as defined above.

The person skilled in the art, from its general knowledge, will know how to select the promoter based on the nucleic sequence to be expressed and/or the host cell, preferably the strain according to the invention, for example the one filed with the CNCM (Collection Nationale de Culture de Microorganismes, Institut Pasteur, 25 rue du Docteur Roux, 75724 Paris Cedex 15, France), under number CNCM I-5499, or under number CNCM I-5681, or under number CNCM I-5682.

In the present invention, the insertion of the nucleic acid sequence encoding the transport protein or permease can be carried out by any process as described above.

Advantageously, when the strain further comprises an expression vector allowing the expression of at least one gene encoding a transport protein or permease, the strain according to the invention can advantageously internalize non-phosphorylated sugars which can be present in the culture medium.

Advantageously, as mentioned above, the inventors have surprisingly demonstrated that the strain according to the invention advantageously allows to accumulate oligosaccharide synthesis precursors, and can advantageously be used as a support and/or means for the production of oligosaccharides.

As mentioned above, the inventors have surprisingly demonstrated that the strain according to the invention can advantageously allow after production of oligosaccharides a diffusion or excretion of said produced oligosaccharides in the culture medium.

In addition, advantageously the diffusion or excretion of the oligosaccharides in the extracellular medium could allow to obtain a reaction catalyzed by the glycoside-phosphorylases in the direction of the reverse phosphorolysis.

The present invention therefore also relates to the in vitro use of a strain according to the invention for producing oligosaccharides and/or in a process for producing oligosaccharides.

In the present invention, the strain can be used in any in vitro, in particular in cellulo, process, for the production of oligosaccharides, known to the person skilled in the art. It may be any process for producing oligosaccharides comprising an enzymatic synthesis and/or using at least one microorganism. It may be any known process for producing oligosaccharides comprising an enzymatic synthesis and/or using at least one microorganism in which the enzyme or the microorganism can be replaced by the strain according to the invention. It may be, for example, a process for producing oligosaccharides comprising an enzymatic synthesis and/or using at least one microorganism, for example as described in the document: Tathiana Souza Martins Meyer, Ângelo Samir Melim Miguel, Daniel Ernesto Rodríguez Fernańdez and Gisela Maria Dellamora Ortiz “Biotechnological Production of Oligosaccharides Applications in the Food Industry” Oct. 22nd 2015 [17], Erju Tang, Xialin Shen, Jia Wang, Xinxiao Sun, Qipeng Yuan, “Synergetic utilization of glucose and glycerol for efficient myo-inositol biosynthesis” Biotechnology and Bioengineering 2020 [32], Constantin Ruprecht, Friedericke Bönisch, Nele Ilmbergera, Tanja V. Heyera, Erhard T. K. Haupt, Wolfgang R. Streit, Ulrich Rabausch “High level production of flavonoid rhamnosides by metagenome-derived Glycosyltransferase C in Escherichia coli utilizing dextrins of starch as a single carbon source”, Metabolic Engineering Volume 55, September 2019, Pages 212-219 [33], Tatiana Antoine, Alain Heyraud Dr., Claude Bosso Dr., Eric Samain Dr “Highly Efficient Biosynthesis of the Oligosaccharide Moiety of the GD3 Ganglioside by Using Metabolically Engineered Escherichia coli”, Angewandte Chemie, Volume117, Issue9, Feb. 18, 2005 Pages 1374-1376 [34].

The inventors have also surprisingly demonstrated that the strain according to the invention advantageously allows to produce oligosaccharides and to excrete them in the culture medium. In addition, the inventors have demonstrated that the strain according to the invention can allow continuous production/bioproduction of oligosaccharides.

Advantageously, the present invention can allow the production of oligosaccharides, it may be, for example, the oligosaccharides mentioned in Table 3 below. For example, the present invention can allow the production of oligosaccharides based on the glycoside-phosphorylase used as mentioned in Table 3 below.

TABLE 3
PRODUCED OLIGOSACCHARIDES BASED ON THE GLYCOSIDE-
PHOSPHORYLASE

| GenBank | Glycoside-phosphorylase | Product |
|---|---|---|
| ABX41399.1 | 3-O-α-glucopyranosyl-L-rhamnose | 3-O-α- |
| | phosphorylase (Cphy_1019) | glucopyranosyl-L- |
| | | rhamnose |
| ABX42289.1 | D-galactosyl-1,4-L-rhamnose | D-galactosyl-1,4-L- |
| | phosphorylase (Cphy_1920) | rhamnose |
| ACB74662.1 | D-galactosyl-β-1,4-L-rhamnose | D-galactosyl-β-1,4- |
| | phosphorylase (GalRhaP;Oter 1377) | L-rhamnose |
| ADI00307.1 | 1,2-alpha-glucosylglycerol | 1,2-alpha- |
| | phosphorylase(Bsel_2816) | glucosylglycerol |
| BAB97299.1 | trehalose phosphorylase (TreP) | trehalose |
| BAC20640.1 | trehalose phosphorylase (TPase) | trehalose |
| AAF22230.1 | trehalose phosphorylase | trehalose |
| ABC84380.1 | trehalose phosphorylase | trehalose |
| BAA31350.1 | trehalose phosphorylase | trehalose |
| AAS 19693.1 | β-1,4-mannosyl-glucose | β-1,4-mannosyl- |
| | phosphorylase (Unk1) | glucose |
| ABY93073.1 | β-1,2-oligomannan phosphorylase | β-1,2-oligomannan |
| | (Teth514_1788) | |
| ABY93074.1 | β-1,2-mannobiose phosphorylase | β-1,2-mannobiose |
| | (Teth514_1789) | |
| ADD61463.1 | β-1,4-mannopyranosyl-[N-glycan] | β-1,4- |
| | phosphorylase (Uhgb_MP) | mannooligosaccharide |
| ADU20661.1 | β-1,4-mannooligosaccharide | β-1,4- |
| | phosphorylase | mannooligosaccharide |
| | (MOP;RaMP2;Rumal_0099) | |
| ADU21379.1 | β-1,4-mannosyl-glucose | β-1,4-mannosyl- |
| | phosphorylase | glucose |
| | (RaMP1;RaMGP;Rumal_0852) | |
| CAC96089.1 | β-1,2-mannobiose phosphorylase | β-1,2-mannobiose |
| | (Lin0857) | |
| CAH06518.1 | β-1,4-mannosyl-glucose | β-1,4-mannosyl- |
| | phosphorylase (MGP;BF0772) | glucose |
| CAZ94304.1 | β-1,3-mannooligo saccharide | β-1,3- |
| | phosphorylase (zobellia_231) | mannooligosaccharide |
| WP_026485574.1 | β-1,4-mannooligosaccharide | β-1,4- |
| | phosphorylase (CalpoDRAFT_0075) | mannooligosaccharide |
| WP_026486530.1 | β-1,4-mannooligosaccharide | β-1,4- |
| | phosphorylase (CalpoDRAFT_1209) | mannooligosaccharide |
| VCV21229.1 | β-1,4-mannooligosaccharide | β-1,4- |
| | phosphorylase | mannooligosaccharide |
| | (RIL182_01100;ROSINTL182_ | |
| | 05474) | |
| VCV21228.1 | Mannosyl-glucose phosphorylase | β-1,4-mannosyl- |
| | (RIL182_01099;ROSINTL182_ | glucose |
| | 07685) | |
| XP_003872966.1 | β-1,2-oligomannan phosphorylase | β-1,2-oligomannan |
| CBZ24448.1 | MTP3 (LMXM_10_1250) | |
| CBZ24449.1 | β-1,2-oligomannan phosphorylase | β-1,2-oligomannan |
| XP_003872967.1 | MTP4 (LMXM_10_1260) | |
| CBZ24451.1 | β-1,2-oligomannan phosphorylase | β-1,2-oligomannan |
| XP_003872969.1 | MTP6 (LMXM_10_1280) | |
| CBZ24452.1 | β-1,2-oligomannan phosphorylase | β-1,2-oligomannan |
| XP_003872970.1 | MTP7 (LMXM_10_1290) | |
| ABX81345.1 | laminaribiose phosphorylase | laminaribiose |
| | (ACL_0729) | |
| BAJ10826.1 | laminaribiose phosphorylase (LbpA) | laminaribiose |
| GigaDB accession | Glycoside-phosphorylase | Product |
| number (GigaDB, DOI: | | |
| 10.5524/100064) | | |
| MH0373_GL0093988 | β-1,4-mannooligosaccharide | β-1,4- |
| | phosphorylase | mannooligosaccharide |
| 340101.Vvad_PD3074 | β-1,3-mannosyl-glucose | β-1,3-mannosyl- |
| | phosphorylase/ | glucose |
| | β-1,3-mannooligo saccharide | |
| | phosphorylase | |
| 340101.Vvad_PD3074 | β-1,3-mannosyl-glucose | β-1,3- |
| | phosphorylase/β-1,3- | mannooligosaccharide |
| | mannooligosaccharide phosphorylase | |
| MH0431_GL0150624 | β-1,4-mannosyl-glucuronate | β-1,4-mannosyl- |
| | phosphorylase | glucuronate |

The present invention therefore also relates to an in vitro, in particular in cellulo, process for producing oligosaccharides comprising the steps of
a) transformation of a strain according to the invention with an expression vector of an enzyme, preferably a glycoside-phosphorylase,
b) culture of the transformed strain in a culture medium,
c) recovery of the produced oligosaccharides.

The present invention also relates to an in vitro, in particular in cellulo, process for producing oligosaccharides comprising the steps of

a) transformation of the strain filed with the CNCM (Collection Nationale de Culture de Microorganismes, Institut Pasteur, 25 rue du Docteur Roux, 75724 Paris Cedex 15, France), under number CNCM I-5499 with an expression vector of an enzyme, preferably a glycoside-phosphorylase,

b) culture of the transformed strain in a culture medium,

c) recovery of the produced oligosaccharides.

The present invention further relates to an in vitro, in particular in cellulo, process for producing oligosaccharides comprising the steps of

a) transformation of the strain filed with the CNCM (Collection Nationale de Culture de Microorganismes, Institut Pasteur, 25 rue du Docteur Roux, 75724 Paris Cedex 15, France), under number CNCM I-5499 further comprising the Δmaa and ΔmanA mutations, with an expression vector of an enzyme, preferably a glycoside-phosphorylase,

b) culture of the transformed strain in a culture medium,

c) recovery of the produced oligosaccharides.

The present invention also further relates to an in vitro, in particular in cellulo, process for producing oligosaccharides comprising the steps of

a) transformation of the strain filed with the CNCM (Collection Nationale de Culture de Microorganismes, Institut Pasteur, 25 rue du Docteur Roux, 75724 Paris Cedex 15, France), under number CNCM I-5681 with an expression vector of an enzyme, preferably a glycoside-phosphorylase,

b) culture of the transformed strain in a culture medium,

c) recovery of the produced oligosaccharides.

The present invention also further relates to an in vitro, in particular in cellulo, process for producing oligosaccharides comprising the steps of

a) transformation of the strain filed with the CNCM (Collection Nationale de Culture de Microorganismes, Institut Pasteur, 25 rue du Docteur Roux, 75724 Paris Cedex 15, France), under number CNCM I-5682 with an expression vector of an enzyme, preferably a glycoside-phosphorylase,

b) culture of the transformed strain in a culture medium,

c) recovery of the produced oligosaccharides.

In the present invention, “transfection”, “transformation” or “transduction” denotes the introduction of one or more exogenous polynucleotides into a host cell by physical or chemical methods. It may be any suitable transfection and/or transformation and/or transduction method known to the person skilled in the art. It may be, for example, a process described in Murray E. J. (ed.), Methods in Molecular Biology, Vol. 7, Gene Transfer and Expression Protocols, Humana Press (1991) [25]) namely a coprecipitation of DNA calcium phosphate, DEAE-dextran; electroporation; cationic liposomes transfection; liposome transfection; facilitated microparticle bombardment of tungsten particles (Johnston, Nature, 346: 776-777 (1990) [26]); and the DNA co-precipitation of strontium phosphate (Brash et al., Mol. Cell Biol. 7: 2031-2034 (1987) [28]). In some cases, lipofection, nucleofection, or temporary membrane disruption (for example electroporation or distortion) can be used to introduce one or more exogenous polynucleotides into the host cell.

In the present invention, the expression vector may be as described above.

In the present invention, the enzyme may be as described above. For example, it can be a glycoside-phosphorylase as described above.

According to the invention, the process may further comprise a step a′) of transformation of a strain according to the invention with an expression vector of at least one transport protein or permease.

According to the invention, the process may also further comprise a step a′) of transformation of the strain filed with the CNCM (Collection Nationale de Culture de Microorganismes, Institut Pasteur, 25 rue du Docteur Roux, 75724 Paris Cedex 15, France), under number CNCM I-5499 with an expression vector of at least one transport protein or permease.

According to the invention, when the strain used in the process is the strain filed with the CNCM under the number CNCM I-5499 further comprising the Δmaa and ΔmanA mutations, the process may further comprise a step a′) of transformation of the strain filed with the CNCM (Collection Nationale de Culture de Microorganismes, Institut Pasteur, 25 rue du Docteur Roux, 75724 Paris Cedex 15, France), under number CNCM I-5499 further comprising the Δmaa and ΔmanA mutations with an expression vector of at least one transport protein or permease.

According to the invention, when the strain used in the process is the strain filed with the CNCM under the number CNCM I-5681, the process may also further comprise a step a′) of transformation of the strain filed with the CNCM (Collection Nationale de Culture de Microorganismes, Institut Pasteur, 25 rue du Docteur Roux, 75724 Paris Cedex 15, France), under number CNCM I-5681 with an expression vector of at least one transport protein or permease.

According to the invention, when the strain used in the process is the strain filed with the CNCM under the number CNCM I-5682, the process may also further comprise a step a′) of transformation of the strain filed with the CNCM (Collection Nationale de Culture de Microorganismes, Institut Pasteur, 25 rue du Docteur Roux, 75724 Paris Cedex 15, France), under number CNCM I-5682 with an expression vector of at least one transport protein or permease.

In the present invention, the expression vector may be as described above.

In the present invention, the transport protein or permease may be as described above. For example, it may be the galactose permease as described above.

According to the invention steps a) and a′) can be successive or concomitant.

In the present invention, the strain culture step can be carried out by any suitable process and/or method known to the person skilled in the art.

It may be, for example, a continuous (“continuous fermentation”), fed-batch (“fed-batch fermentation”) or batch (“batch fermentation”) culture process.

In the present invention, the culture step may be a continuous culture or a culture for a period of time. For example, the culture can be carried out over a period of 1 to 12 hours, 1 to 24 hours, 1 to 36 hours, 1 to 48 hours, 1 to 240 hours. For example, the culture step can be carried out until confluence of the strain in the medium.

In the present invention, the strain culture step can be carried out in any suitable container and/or device for the culture of microorganisms, preferably bacteria, known to the person skilled in the art. It may be, for example, a commercially available culture reactor, for example a Multifors bioreactor (Infors, Switzerland).

In the present invention, the culture medium can be inoculated from a preculture of the strain, said preculture having an optical density (OD) at 600 nm of between 0.01 and 1, preferably of 0.1.

In the present invention, the culture medium can be inoculated with a strain concentration of 2.107 to 2.1010 bacteria per mL, preferably 2.108 bacteria per mL of culture medium.

According to the invention, the culture medium can be any suitable culture medium known to the person skilled in the art. It may be, for example, a liquid or solid medium, preferably a liquid medium. It may be any rich medium known to the person skilled in the art. It may be, for example, the LB (Lysogeny Broth), Superbroth, TB (Terrific Broth), YPD (Yeast Extract-Peptone Dextrose) medium. It may be, for example, any suitable minimum medium known to the person skilled in the art, for example an A medium, the M9 or M63 medium supplemented with an adequate carbon source, for example glucose, galactose and/or mannose. It may be, for example, suitable selective media known to the person skilled in the art, for example the YNB (Yeast Nitrogen Base) medium.

It may preferably be a liquid bacteria culture medium. It may be, for example, a commercially available liquid culture medium, for example the M9 medium commercialized by the Thermofischer company, the Terrific Broth medium commercialized by the Thermofischer company, the “MagicMedia (trade mark) E. coli Expression Medium” commercialized by the Thermofischer company, the S.O.C. medium. commercialized by the Thermofischer company.

It may be a medium described in the document Karen L. Elbing Roger Brent, Recipes and Tools for Culture of Escherichia coli, Current Tools, 9 Nov. 2018 [52]

It may preferably be a liquid medium comprising the following components: Na2HPO4 12H2O 17.4 g/L, KH2PO4 3.02 g/L, NaCl 0.51 g/L, NH4C1 2.04 g/L, Na2EDTA 2 H2O 15 mg/L, ZnSO4 7 H2O 4.5 mg/L, CoCl2 6H2O 0.3 mg/L, MnCl2 4H2O 1 mg/L, H3BO3 1 mg/L, Na2MoO4 2 H2O 0.4 mg/L, FeSO4 7 H2O 3 mg/L, CuSO4 5 H2O 0.3 mg/L. MgSO4 0.5 g/L CaCl2 4.38 mg/L, Thiamine hypochloride 0.1 g/L, or a liquid medium comprising the following components: KH2PO4 3.02 g/L, NaCl 0.51 g/L, NH4C1 2.04 g/L, (NH4)2SO4 5 g/L, Na2EDTA 2 H2O15 mg/L, ZnSO4 7 H2O4.5 mg/L, CoCl2 6H2O0.3 mg/L, MnCl2 4H2O1 mg/L, H3BO3 1 mg/L, Na2MoO4 2 H2O0.4 mg/L, FeSO4 7 H2O3 mg/L, CuSO4 5 H2O0.3 mg/L. MgSO4 0.5 g/L CaCl₂) 4.38 mg/L, Thiamine hypochloride 0.1 g/L.

In the present invention, the culture medium may further comprise at least one phosphorylated monosaccharide. For example, the culture medium may comprise at least one phosphorylated monosaccharide selected from the group comprising glucose 1-Phosphate, N-acetyl-α-d-glucosamine 1-Phosphate, Galactose 1-Phosphate, mannose 1-Phosphate; or any mixture thereof.

In the present invention, the phosphorylated monosaccharide can be present at a concentration ranging from 1 to 200 g·L−1, preferably from 5 to 200 g·L−1 of culture medium.

In the present invention, the culture medium may further comprise at least one phosphorylated monosaccharide based on the glycoside-phosphorylase expressed by the expression vector as presented in Table 4 below:

TABLE 4
PHOSPHORYLATED MONOSACCHARIDE BASED ON THE
GLYCOSIDE-PHOSPHORYLASE USED

| | | Phosphorylated |
|---|---|---|
| GenBank | Glycoside-phosphorylase | monosaccharide |
| ABX41399.1 | 3-O-α-glucopyranosyl-L-rhamnose | Glucose 1-P |
| | phosphorylase (Cphy_1019) | |
| ABX42289.1 | D-galactosyl-1,4-L-rhamnose | Galactose 1-P |
| | phosphorylase (Cphy_1920) | |
| ACB74662.1 | D-galactosyl-β-1,4-L-rhamnose | Galactose 1-P |
| | phosphorylase (GalRhaP;Oter 1377) | |
| ADI00307.1 | 1,2-alpha-glucosylglycerol | Glucose 1-P |
| | phosphorylase(Bsel_2816) | |
| BAB97299.1 | trehalose phosphorylase (TreP) | Glucose 1-P |
| BAC20640.1 | trehalose phosphorylase (TPase) | Glucose 1-P |
| AAF22230.1 | trehalose phosphorylase | Glucose 1-P |
| ABC84380.1 | trehalose phosphorylase | Glucose 1-P |
| BAA31350.1 | trehalose phosphorylase | Glucose 1-P |
| ABX81345.1 | laminaribiose phosphorylase | Glucose 1-P |
| | (ACL_0729) | |
| BAJ10826.1 | laminaribiose phosphorylase (LbpA) | Glucose 1-P |
| AAS19693.1 | β-1,4-mannosyl-glucose | Mannose 1-P |
| | phosphorylase (Unk1) | |
| ABY93073.1 | β-1,2-oligomannan phosphorylase | Mannose 1-P |
| | (Teth514_1788) | |
| ABY93074.1 | β-1,2-mannobiose phosphorylase | Mannose 1-P |
| | (Teth514_1789) | |
| ADD61463.1 | β-1,4-mannopyranosyl-[N-glycan] | Mannose 1-P |
| | phosphorylase (Uhgb_MP) | |
| ADU20661.1 | β-1,4-mannooligosaccharide | Mannose 1-P |
| | phosphorylase | |
| | (MOP;RaMP2;Rumal_0099) | |
| ADU21379.1 | β-1,4-mannosyl-glucose | Mannose 1-P |
| | phosphorylase | |
| | (RaMP1;RaMGP;Rumal_0852) | |
| CAC96089.1 | β-1,2-mannobiose phosphorylase | Mannose 1-P |
| | (Lin0857) | |
| CAH06518.1 | β-1,4-mannosyl-glucose | Mannose 1-P |
| | phosphorylase (MGP;BF0772) | |
| CAZ94304.1 | β-1,3-mannooligosaccharide | Mannose 1-P |
| | phosphorylase (zobellia_231) | |
| WP_026485574.1 | β-1,4-mannooligosaccharide | Mannose 1-P |
| | phosphorylase (CalpoDRAFT_0075) | |
| WP_026486530.1 | β-1,4-mannooligosaccharide | Mannose 1-P |
| | phosphorylase (CalpoDRAFT_1209) | |
| VCV21229.1 | β-1,4-mannooligosaccharide | Mannose 1-P |
| | phosphorylase | |
| | (RIL182_01100;ROSINTL182_ | |
| | 05474) | |
| VCV21228.1 | Mannosyl-glucose phosphorylase | Mannose 1-P |
| | (RIL182_01099;ROSINTL182_ | |
| | 07685) | |
| XP_003872966.1 | β-1,2-oligomannan phosphorylase | Mannose 1-P |
| CBZ24448.1 | MTP3 (LMXM_10_1250) | |
| CBZ24449.1 | β-1,2-oligomannan phosphorylase | Mannose 1-P |
| XP_003 872967.1 | MTP4 (LMXM_10_1260) | |
| CBZ24451.1 | β-1,2-oligomannan phosphorylase | Mannose 1-P |
| XP_003872969.1 | MTP6 (LMXM_10_1280) | |
| CBZ24452.1 | β-1,2-oligomannan phosphorylase | Mannose 1-P |
| XP_003 872970.1 | MTP7 (LMXM_10_1290) | |
| GigaBase accession | Glycoside-phosphorylase | Phosphorylated |
| number (GigaDB, DOI: | | monosaccharide |
| 10.5524/100064) | | |
| MH0373_GL0093988 | β-1,4-mannooligosaccharide | Mannose 1-P |
| | phosphorylase | |
| 340101.Vvad_PD3074 | β-1,3-mannosyl-glucose | Mannose 1-P |
| | phosphorylase/β-1,3- | |
| | mannooligosaccharide phosphorylase | |
| 340101.Vvad_PD3074 | β-1,3-mannosyl-glucose | Mannose 1-P |
| | phosphorylase/β-1,3- | |
| | mannooligosaccharide phosphorylase | |
| MH0431_GL0150624 | β-1,4-mannosyl-glucuronate | Mannose 1-P |
| | phosphorylase (“β-1,4-mannosyl- | |
| | glucuronic acid phosphorylase”) | |

Advantageously, the inventors have demonstrated that when the strain further comprises an expression vector allowing the expression of at least one gene encoding a non-PTS transport protein or permease, the strain according to the invention can advantageously internalize non-phosphorylated carbohydrates which can be present in the culture medium.

Also, the process according to the invention can advantageously allow to use non-phosphorylated carbohydrates and to break free from the costly use of usual phosphorylated monosaccharides and/or phosphorylated monosaccharides as mentioned in table 4 above.

According to the invention, the culture medium may further comprise at least one non-phosphorylated carbohydrate, preferably one monosaccharide. For example, the culture medium may comprise at least one non-phosphorylated carbohydrate selected from the group comprising d-glucose, L-rhamnose, glycerol, d-mannose or any mixture thereof.

In the present invention, the concentration in the culture medium of said at least one non-phosphorylated carbohydrate can be from 1 to 100 g·L−1, preferably from 2 to 200 g·L−1, preferably from 1 to 5 g·L−1 of culture medium, in one or more successive additions.

In the present invention, the culture medium may further comprise at least one non-phosphorylated monosaccharide based on the glycoside-phosphorylase expressed by the expression vector, for example, as presented in Table 5 below:

TABLE 5
NON-PHOSPHORYLATED CARBOHYDRATES BASED ON
THE GLYCOSIDE-PHOSPHORYLASE

| | | Non- |
|---|---|---|
| | | phosphorylated |
| GenBank | Glycoside-phosphorylase | carbohydrates |
| ABX41399.1 | 3-O-α-glucopyranosyl-L-rhamnose | L-rhamnose |
| | phosphorylase (Cphy_1019) | |
| ABX42289.1 | D-galactosyl-1,4-L-rhamnose | L-rhamnose |
| | phosphorylase (Cphy_1920) | |
| ACB74662.1 | D-galactosyl-β-1,4-L-rhamnose | L-rhamnose |
| | phosphorylase (GalRhaP;Oter_1377) | |
| ADI00307.1 | 1,2-alpha-glucosylglycerol | glycerol |
| | phosphorylase(Bsel_2816) | |
| BAB97299.1 | trehalose phosphorylase (TreP) | D-glucose |
| BAC20640.1 | trehalose phosphorylase (TPase) | D-glucose |
| AAF22230.1 | trehalose phosphorylase | D-glucose |
| ABC84380.1 | trehalose phosphorylase | D-glucose |
| BAA31350.1 | trehalose phosphorylase | D-glucose |
| AAS19693.1 | β-1,4-mannosyl-glucose | D-glucose |
| | phosphorylase (Unk1) | |
| ABX81345.1 | laminaribiose phosphorylase | D-glucose |
| | (ACL_0729) | |
| BAJ10826.1 | laminaribiose phosphorylase (LbpA) | D-glucose |
| ABY93073.1 | β-1,2-oligomannan phosphorylase | D-mannose |
| | (Teth514_1788) | |
| ABY93074.1 | β-1,2-mannobiose phosphorylase | D-mannose |
| | (Teth514_1789) | |
| ADD61463.1 | β-1,4-mannopyranosyl-[N-glycan] | D-mannose |
| | phosphorylase (Uhgb_MP) | |
| ADU20661.1 | 3-1,4-mannooligosaccharide | D-mannose |
| | phosphorylase | |
| | (MOP;RaMP2;Rumal_0099) | |
| ADU21379.1 | β-1,4-mannosyl-glucose | D-glucose |
| | phosphorylase | |
| | (RaMP1;RaMGP;Rumal_0852) | |
| CAC96089.1 | β-1,2-mannobiose phosphorylase | D-mannose |
| | (Lin0857) | |
| CAH06518.1 | β-1,4-mannosyl-glucose | D-glucose |
| | phosphorylase (MGP;BF0772) | |
| CAZ94304.1 | β-1,3-mannooligo saccharide | D-mannose |
| | phosphorylase (zobellia_231) | |
| WP_026485574.1 | β-1,4-mannooligosaccharide | D-mannose |
| | phosphorylase (CalpoDRAFT_0075) | |
| WP_026486530.1 | β-1,4-mannooligosaccharide | D-mannose |
| | phosphorylase (CalpoDRAFT_1209) | |
| VCV21229.1 | β-1,4-mannooligosaccharide | D-mannose |
| | phosphorylase | |
| | (RIL182_01100;ROSINTL182_ | |
| | 05474) | |
| VCV21228.1 | Mannosyl-glucose phosphorylase | D-glucose |
| | (RIL182_01099;ROSINTL182_ | |
| | 07685) | |
| XP_003872966.1 | β-1,2-oligomannan phosphorylase | D-mannose |
| CBZ24448.1 | MTP3 (LMXM_10_1250) | |
| CBZ24449.1 | β-1,2-oligomannan phosphorylase | D-mannose |
| XP_003872967.1 | MTP4 (LMXM_10_1260) | |
| CBZ24451.1 | β-1,2-oligomannan phosphorylase | D-mannose |
| XP_003872969.1 | MTP6 (LMXM_10_1280) | |
| CBZ24452.1 | β-1,2-oligomannan phosphorylase | D-mannose |
| XP_003872970.1 | MTP7 (LMXM_10_1290) | |
| GigaDB accession | Glycoside-phosphorylase | Non- |
| number (GigaDB, DOI: | | phosphorylated |
| 10.5524/100064) | | carbohydrates |
| MH0373_GL0093988 | β-1,4-mannooligosaccharide | D-mannose |
| | phosphorylase | |
| 340101.Vvad_PD3074 | β-1,3-mannosyl-glucose | D-glucose |
| | phosphorylase/β-1,3- | |
| | mannooligosaccharide phosphorylase | |
| 340101.Vvad_PD3074 | β-1,3-mannosyl-glucose | D-mannose |
| | phosphorylase/β-1,3- | |
| | mannooligosaccharide phosphorylase | |
| MH0431_GL0150624 | 3-1,4-mannosyl-glucuronate | D-glucuronic acid |
| | phosphorylase (“β-1,4-mannosyl- | |
| | glucuronic acid phosphorylase”) | |

In the present invention, during the culture step, the process may further comprise at least one step of adding at least one non-phosphorylated carbohydrate to the culture medium.

It may be at least one non-phosphorylated carbohydrate as mentioned above.

In the present invention, the culture step can be carried out at a temperature of 25 to 45° C., for example of 35 to 38° C., for example of 37° C.

In the present invention, the recovery of the produced oligosaccharides can be carried out in the culture medium or in the strain, preferably in the culture medium.

In the present invention, the step of recovering oligosaccharides can be carried out by any suitable process known to the person skilled in the art. It may be, for example, a step comprising or not one or more purification steps. It may be, for example, a process described in the document Israel Pedruzzi, Eduardo Borges da Silva, Alirio E Rodrigues “Selection of resins, equilibrium and sorption kinetics of lactobionic acid, fructose, lactose and sorbitol” Separation and Purification Technology 2008 Volume 63, Issue 3, 3 Nov. 2008, Pages 600-611 [44], Clarisse Nobre, José A. Teixeira & Lígia R. Rodrigues (2015) “New Trends and Technological Challenges in the Industrial Production and Purification of Fructo-oligosaccharides”, Critical Reviews in Food Science and Nutrition, 55:10, 1444-1455, 11 Oct. 2013 [45] and/or the internet page https://www.novasep.com/technologies/industrial-technologies-for-evaporation-concentration-and-crystallization.html [46].

It may be, for example, a step comprising a microfiltration step, an ultrafiltration step, a nanofiltration step, a reverse osmosis step, a lyophilization step, an evaporation step, for example on a rotative evaporator, an atomization step, a liquid phase extraction step, a product crystallization step, a chromatography step, a dialysis and/or electrodialysis step.

It may be, for example, a step comprising a filtration of the medium through a filter, for example with pores of 0.3 nm to 10 μm in diameter, for example with pores of 0.1 to 10 μm in diameter for a microfiltration, of 1 to 100 nm in diameter for an ultrafiltration, or of 0.3 to 1 nm in diameter for a nanofiltration.

It may be, for example, a process comprising a chromatography, for example in gas phase, in liquid or ionic phase, an electrophoresis, for example a capillary electrophoresis, a filtration, for example a nanofiltration, a solid phase extraction, a solid phase micro-extraction, a solvent extraction. It may be, for example, a liquid phase chromatography, a high performance liquid chromatography, a liquid-solid chromatography, a liquid-liquid chromatography, a reverse-polarity partition chromatography, an ion exchange chromatography, an ionic chromatography, an ionic interaction chromatography, a hydrophobic interaction chromatography, a size exclusion chromatography, for example with a resin of the Sephadex G15 or Biogel P2 type, a gel permeation chromatography, a gel filtration chromatography, a ligand exchange chromatography, a forced flow fractionation, a planar chromatography, a centrifugal partition chromatography, a counter-current chromatography, a centrifugal liquid-liquid chromatography, a chiral stationary phase chromatography.

The person skilled in the art, from its general knowledge, will know how to adapt and/or select the recovery process based on the medium and/or the produced oligosaccharide.

Advantageously, the process according to the invention can allow the production of oligosaccharides, for example the oligosaccharides mentioned in table 6 below. For example, the present invention can allow the production of oligosaccharides based on the glycoside-phosphorylase used as mentioned in Table 6 below.

TABLE 6
PRODUCED OLIGOSACCHARIDES BASED ON THE GLYCOSIDE-
PHOSPHORYLASE

| GenBank | Glycoside-phosphorylase | Product |
|---|---|---|
| ABX41399.1 | 3-O-α-glucopyranosyl-L- | 3-O-α-glucopyranosyl- |
| | rhamnose phosphorylase | L-rhamnose |
| | (Cphy_1019) | |
| ABX42289.1 | D-galactosyl-1,4-L-rhamnose | D-galactosyl-1,4-L- |
| | phosphorylase (Cphy_1920) | rhamnose |
| ACB74662.1 | D-galactosyl-β-1,4-L-rhamnose | D-galactosyl-β-1,4-L- |
| | phosphorylase | rhamnose |
| | (GalRhaP;Oter_1377) | |
| ADI00307.1 | 1,2-alpha-glucosylglycerol | 1,2-alpha- |
| | phosphorylase(Bsel_2816) | glucosylglycerol |
| BAB97299.1 | trehalose phosphorylase (TreP) | trehalose |
| BAC20640.1 | trehalose phosphorylase (TPase) | trehalose |
| AAF22230.1 | trehalose phosphorylase | trehalose |
| ABC84380.1 | trehalose phosphorylase | trehalose |
| BAA31350.1 | trehalose phosphorylase | trehalose |
| AAS19693.1 | β-1,4-mannosyl-glucose | β-1,4-mannosyl-glucose |
| | phosphorylase (Unk1) | |
| ABY93073.1 | β-1,2-oligomannan | β-1,2-oligomannan |
| | phosphorylase (Teth514_ 1788) | |
| ABY93074.1 | β-1,2-mannobiose | β-1,2-mannobiose |
| | phosphorylase (Teth514_1789) | |
| ADD61463.1 | β-1,4-mannopyranosyl-[N- | β-1,4- |
| | glycan] phosphorylase | mannooligosaccharide |
| | (Uhgb_MP) | |
| ADU20661.1 | β-1,4-mannooligosaccharide | β-1,4- |
| | phosphorylase | mannooligosaccharide |
| | (MOP;RaMP2;Rumal_0099) | |
| ADU21379.1 | β-1,4-mannosyl-glucose | β-1,4-mannosyl-glucose |
| | phosphorylase | |
| | (RaMPl;RaMGP;Rumal_0852) | |
| CAC96089.1 | β-1,2-mannobiose | β-1,2-mannobiose |
| | phosphorylase (Lin0857) | |
| CAH06518.1 | β-1,4-mannosyl-glucose | β-1,4-mannosyl-glucose |
| | phosphorylase (MGP;BF0772) | |
| CAZ94304.1 | β-1,3-mannooligosaccharide | β-1,3- |
| | phosphorylase (zobellia_231) | mannooligosaccharide |
| WP_026485574.1 | β-1,4-mannooligosaccharide | β-1,4- |
| | phosphorylase | mannooligosaccharide |
| | (CalpoDRAFT_0075) | |
| WP_026486530.1 | β-1,4-mannooligosaccharide | β-1,4- |
| | phosphorylase | mannooligosaccharide |
| | (CalpoDRAFT_1209) | |
| VCV21229.1 | β-1,4-mannooligosaccharide | β-1,4- |
| | phosphorylase | mannooligosaccharide |
| | (RIL182_01100;ROSINTL182_ | |
| | 05474) | |
| VCV21228.1 | Mannosyl-glucose | β-1,4-mannosyl-glucose |
| | phosphorylase | |
| | (RIL182_01099;ROSINTL182_ | |
| | 07685) | |
| XP_003872966.1 | β-1,2-oligomannan | β-1,2-oligomannan |
| CBZ24448.1 | phosphorylase MTP3 | |
| | (LMXM_10_1250) | |
| CBZ24449.1 | β-1,2-oligomannan | β-1,2-oligomannan |
| XP_003872967.1 | phosphorylase MTP4 | |
| | (LMXM_10_1260) | |
| CBZ24451.1 | β-1,2-oligomannan | β-1,2-oligomannan |
| XP_003872969.1 | phosphorylase MTP6 | |
| | (LMXM_10_1280) | |
| CBZ24452.1 | β-1,2-oligomannan | β-1,2-oligomannan |
| XP_003872970.1 | phosphorylase MTP7 | |
| | (LMXM_10_1290) | |
| ABX81345.1 | laminaribiose phosphorylase | laminaribiose |
| | (ACL_0729) | |
| BAJ10826.1 | laminaribiose phosphorylase | laminaribiose |
| | (LbpA) | |
| GigaB | Glycoside-phosphorylase | Product |
| (GigaDB, DOI: | | |
| 10.5524/100064) | | |
| MH0373_GL0093988 | β-1,4-mannooligosaccharide | β-1,4- |
| | phosphorylase | mannooligosaccharide |
| 340101.Vvad_PD3074 | β-1,3-mannosyl-glucose | β-1,3-mannosyl-glucose |
| | phosphorylase/β-1,3- | |
| | mannooligosaccharide | |
| | phosphorylase | |
| 340101.Vvad_PD3074 | β-1,3-mannosyl-glucose | β-1,3- |
| | phosphorylase/β-1,3- | mannooligosaccharide |
| | mannooligosaccharide | |
| | phosphorylase | |
| MH0431_GL0150624 | β-1,4-mannosyl-glucuronate | β-1,4-mannosyl- |
| | phosphorylase (“β-1,4- | glucuronate |
| | mannosyl-glucuronic acid | |
| | phosphorylase”) | |

Other advantages may be seen by the person skilled in the art by reading the following examples, shown by the appended figures provided by way of illustration.

### BRIEF DESCRIPTION OF THE FIGURES

FIG. 1 is a picture showing the PCR products of DNA fragments amplified from the genome of the MDO, MGX and PFKA strains, and having migrated on an agarose gel. Agarose gel of PCR products of the MDO, MGX and PFKA strains. Tracks 1 and 5, size marker; tracks 2, 3 and 4, PCR products of the ptsG gene fragment; tracks 6, 7 and 8, PCR products of the manXYG operon fragment.

FIG. 2 is a picture showing the PCR products of DNA fragments amplified from the genome of MDO, MGX and PFKA strains, and having migrated on an agarose gel. Agarose gel of PCR products of the MDO, MGX and PFKA strains. Track 1, size marker; tracks 2, 3 and 4, PCR products of the pfkA gene fragment.

FIG. 3 is a diagram representing the growth curves of the E. coli MDO (diamonds), MGX (light gray squares) and MGX1 (dark gray squares) strains in minimum M9 medium supplemented with 3 g/L of glucose, the ordinate corresponds to the Optical Density measured at 600 nm (OD600) and the abscissa to time in hours (H).

FIG. 4 is a histogram representing a comparison of metabolite concentrations between the E. coli MDO strain and the MGX1 strain (black bars) and between the E. coli MDO strain and the PFKA1 strain (hatched bars). In the figure F6P means Fructose-6-phosphate, FBP means Fructose-1,6-Bisphosphate, G1P/M1P: Glucose-1-Phosphate/Mannose-1-Phosphate, G6P: Glucose-6-Phosphate, Galactose-1P: Galactose-1-Phosphate, GluN-6P:Glucosamine-6-Phosphate, Man-6P: Mannose-6-Phosphate, N-AcGlucoseN-1P: N-acetyl glucose-1-Phosphate, N-AcGlucoseN-6P: N-acetyl glucose-6-Phosphate, Rib-5P/Ribulose-5P: Ribose-5-Phosphate/Ribulose-5-Phosphate, Sed7P sedoheptulose-7-phosphate. The ordinate corresponds to the value of the metabolite concentration ratios expressed in log 2.

FIG. 5 represents pictures of SDS-PAGE electrophoresis gel (FIG. 5 A) and a diagram corresponding to the evolution of the Optical Density in the culture medium as a function of time (FIG. 5 B). FIG. 5 A corresponds to the picture of 7 SDS-PAGE electrophoresis gels carried out at 22 h, 28 h, 46 h, 55 h, 79 h, 101 h and 126 h respectively of culture of the non-transformed PFKA1 strain (control), with the pBAD-Teth1788 or pTRC-Teth-1788 vector. FIG. 5 B is a diagram of the evolution of the Optical Density at 600 nm of the culture medium (ordinate) of the non-transformed PFKA1 strain (control-cross), with the pBAD-Teth1788 (squares) or pTRC-Teth1788 (triangle) vector as a function of time (abscissa).

FIG. 6 corresponds to a diagram of the metabolism of the PFKA1 strain transformed with the pBAD-Teth1788 vector or with the pBAD-Uhgb_MP vector, or with the pBAD-β-1,3-mannoside-phosphorylase vector. In this figure the abbreviations mean: galP: galactose permease, PTS: Phosphotransferases, Glk: Glucokinase, manA: mannose-6-phosphate isomerase, pgi: glucose-6-phosphate isomerase, zwf: Glucose-6-phosphate dehydrogenase, Man-1P: Mannose-1-Phosphate, Man-6P: Mannose-6-Phosphate, Glu-6-P: Glucose-6-Phosphate, Fru-6-P: Fructose-6-Phosphate, FBP: Fructose-1,6-Bisphosphate.

FIG. 7 corresponds to the NMR spectrum of samples of culture medium of PFKA1 strain transformed with the pBAD-Teth1788 vector (upper curve) or of samples of culture medium of non-transformed PFKA1 strain (control).

FIG. 8 corresponds to the chromatogram of a High-Performance Anion Exchange Chromatography with Pulsed Amperometric Detection (HPAEC-PAD) of samples of culture medium of the PFKA1 strain transformed with the pBAD-HisA-Teth1788 vector (bold solid line curve) or samples of culture medium of non-transformed PFKA1 strain (dotted curve) or of purified β-1,2-mannobiose (β-1,2-Man2) (discontinuous dotted curve).

FIG. 9 corresponds to a diagram of the evolution of the concentration in milliMolar (mM) of mannose (triangles), β1,2-mannobiose (circles) and optical density at 600 nm (squares) as a function of time in minutes in the medium culture of PFKA1 strain transformed with pBAD-Teth-1788.

FIG. 10 corresponds to HPAEC-PAD analysis chromatograms of the purification of β-1,2-mannobiose (β-1,2-man2) from a PFKA1-pBAD-Teth-1788 culture supernatant. FIG. 10 A corresponds to the chromatogram obtained by HPAEC-PAD with the PFKA1-pBAD-Teth-1788 culture supernatant before purification. B: Chromatogram of β-1,2-mannobiose after purification. On the chromatograms, the ordinate represents the conductivity in nanoCoulomb (nC) and the abscissa the time in minutes (min).

FIG. 11 is a diagram of the evolution of the optical density at 600 nm of the culture medium (ordinate) of the non-transformed PKA1 strain (control-cross), with the pBAD-UhgbMP (squares) or pTRC-UhgbMP (triangle) vector as a function of time (abscissa).

FIG. 12 is a NMR spectrum of samples of culture medium of PFKA1 strain transformed with the pBAD-UhgbMP vector (intermediate curve) or samples of culture medium of non-transformed PFKA1 strain (control) (lower curve) and a solution of the commercial standard β-1,4-mannobiose (Carbosynth, UK) (upper curve).

FIG. 13 corresponds to the HPAEC-PAD analysis of samples, the thin solid line curve corresponds to the chromatogram of the culture supernatant of PFKA1 strain transformed with pBAD-UhgbMP, the bold solid line curve corresponds to the chromatogram of the culture supernatant of the non-transformed PFKA1 strain, the dotted line curve corresponds to the commercial standard of β-1,4-mannobiose.

FIG. 14 represents the plasmid sequence of the pWKS-galP vector.

FIG. 15 represents the plasmid sequence of the pBAD-Teth1788 vector.

FIG. 16 represents the plasmid sequence of the pBAD-UhgbMP_vector.

FIG. 17 represents a standard curve of the concentration of β-1,2-mannobiose in milligrams per liter (mg·L−1) (abscissa) as a function of the area under the curve in nanoCoulomb (nC) per minute (nC.min), quantified by HPAEC-PAD.

FIG. 18 represents corresponds to the NMR spectrum of samples of commercial δ-1,3-mannobiose, the culture supernatant of PFKA1 strain transformed with the pBAD-β-1,3-mannooligosaccharide phosphorylase plasmid after 3 (t=3 h) or 108 (t=108 h) hours of culture.

FIG. 19 represents the chromatograms of different samples analyzed by HPAEC-PAD, in this figure the solid bold line curve corresponds to the chromatogram of the culture supernatant of the PFKA1 strain transformed with the pBAD-β-1,3-mannooligosaccharide phosphorylase plasmid after 108 hours of culture, the dotted line curve corresponds to the chromatogram of a commercial solution of δ-1,3-mannobiose.

FIG. 20 represents the plasmid sequence of the pBAD-β-1,3-mannooligosaccharide phosphorylase vector.

FIG. 21 represents the plasmid sequence of the pBAD-ACL_0729 vector.

FIG. 22 is a diagram of the evolution of the optical density at 600 nm of the culture medium (ordinate) of the PKA1 strain non-transformed (control-cross) or transformed with the pBAD-ACL0729 vector (circles) as a function of time (abscissa).

FIG. 23 corresponds to the NMR spectra (normalized thanks to the addition of trimethylsilylpropanoic acid) of samples of commercial laminaribiose, the culture supernatant with the PFKA1 strain transformed with the pBAD-ACL0729 plasmid (with or without the addition of commercial laminaribiose) or the culture supernatant with the non-transformed PFKA1 strain after 49 hours of culture.

FIG. 24 represents the chromatograms of various samples analyzed by HPAEC-PAD, the solid line curve corresponds to the chromatogram of the culture supernatant of the PFKA1 strain transformed with pBAD-ACL0729, the dotted line curve corresponds to the chromatogram of the culture supernatant of the non-transformed PFKA1 strain, the dashed line curve corresponds to the commercial standard of laminaribiose.

FIG. 25 corresponds to the NMR spectra of samples of culture medium of the PFKA1 strain (CS0) or samples of culture medium of the CS0Δmaa strain, transformed with the pBAD-Teth1788 vector.

FIG. 26 is a metabolic diagram of the CS1 strain for the production of mannobiose from glycerol and mannose, by decoupling the production of mannobiose from cell growth. In this figure the abbreviations mean: galP: galactose permease, PTS: Phosphotransferases, glk: Glucokinase, maa: maltose acetyltransferase, manA: mannose-6-phosphate isomerase, manB: phosphomannomutase, Man-1P: Mannose-1-Phosphate, Man-6P: Mannose-6-Phosphate, Fru-6-P: Fructose-6-Phosphate, pfkA: ATP-dependent 6-phosphofructokinase isozyme 1, Teth514-1788 (β-1,2-Man_GP): β-1,2-oligomannan phosphorylase, Uhgb_MP (b-1,4-Man_GP): β-1,4-mannobiose-phosphorylase/β-1,4-mannopyranosyl-[N-glycan] phosphorylase, β-1,3-Man_GP: b-1,3-mannobiose-phosphorylase.

FIG. 27 represents the growth curves of the CS0 (diamonds (A)), CS0-galP-Teth1788 (dashes (B)), CS1-galP-Teth1788 (filled circles (C)), CS1 IG-Teth1788 (triangles (D)), CS1-galP-Lin0857 (empty circles (E)) and CS1 IG-galP-Lin0857 (squares (F)) strains. The ordinate corresponds to the Optical Density measured at 600 nm (OD600) and the abscissa to the time in hours (H).

FIG. 28 represents the plasmid sequence of the pBAD-Lin0857 vector.

### EXAMPLES

### Example 1: Manufacture and Characterization of the Strain Filed with the CNCM Under Number CNCM I-5499

In the example below, the strain filed with the CNCM (Collection Nationale de Culture de Microorganismes, Institut Pasteur, 25 rue du Docteur Roux, 75724 Paris Cedex 15, France), under number CNCM I-5499 is also designated PFKA strain.

### 1. Preparation and Production of the PFKA Strain (CNCM N° I-5499)/Phenotypic Validation

The strain filed with the CNCM (Collection Nationale de Culture de Microorganismes, Institut Pasteur, 25 rue du Docteur Roux, 75724 Paris Cedex 15, France), under number CNCM I-5499 was obtained by genetic modification of the Escherichia coli MDO strain.

The E. coli MDO strain, was obtained and characterized in the laboratoire Chimie et Biotechnologie des Oligosaccharides (CBO) (CERMAV-CNRS CS40700, 38041 Grenoble cedex 9, France) from the Escherichia coli ZLKA strain as described in Fierfort and Samain “Genetic engineering of Escherichia coli for the economical production of sialylated oligosaccharides” J Biotechnol. 2008 Apr. 30; 134β-4):261-5. [3].

The E. coli ZLKA strain derives from the E. coli K-12 DH1 (endA1 recA1 gyrA96 thi-1 gLnV44 relA1 hsdR17) strain whose lacZ, nanKETA and lacA genes have been inactivated as described in Fierfort and Samain “Genetic engineering of Escherichia coli for the economical production of sialylated oligosaccharides” J Biotechnol. 2008 Apr. 30; 134β-4):261-5 [3].

The E. coli MDO strain is characterized by the inactivation of the melA, wcaJ and mdoH genes as well as the insertion of the Plac promoter upstream of the gmd gene encoding a GDP-mannose 4,6-dehydratase. As a result, the phosphomannomutase manB gene, which is located downstream of the gmd gene, is inducible by isopropyl β-D-1-thiogalactopyranoside (IPTG). The complete genotype of the MDO strain is as follows: endA1 recA1 gyrA96 thi-1 gLnV44 relA1 hsdR17 lacZ-wcaF, Plac nanKETA lacA melA wcaJ mdoH.

In the present example, kanamycin and ampicillin were used at a final concentration of 50 μg/mL to ensure remaining of the pWKS-GalP and pBAD-Teth-1788 or pBAD-Uhgb_MP plasmids respectively in the cells. The final concentration of IPTG used for the induction of galP and manB was 60 μM. The final concentration of L-arabinose for the induction of the gene encoding the glycoside-phosphorylase (Teth-1788, UhgbMP or δ-1,3-mannooligosaccharide phosphorylase) was 10 mM. The IPTG was added to the culture medium at the time of inoculation and the L-arabinose is added to the culture at the start of the exponential growth phase (DO₆₀₀≈0.4).

### 1.1. Insertion of the Plac Promoter Upstream of the GMD Gene

A DNA fragment of 302 base pairs (bp) including the sequence of the Plac expression promoter was integrated into the chromosome between the stop codon of the wcaF gene and the start codon of the gmd gene. Two DNA sequences, a 0.88 kb segment ending 5 bp downstream of the stop codon of the wcaF gene and a 0.96 kb segment starting 13 bp upstream from the start codon of the gmd gene were amplified by PCR from the genomic DNA sequence of E. Coli K-12, used as a template. The PCR cycle was 1 cycle of 30 seconds (s) at 98° C., 35 cycles of 15 s at 98° C., 15 s at 69° C. and 30 s at 72° C., 1 cycle of 5 minutes at 72° C. The Polymerase used was Phusion, (NEB). The PL1 (5′ CTCGAGAGGCGATATTTTTCCCTGTATCC SEQ ID NO 1) and PL2 (5′GGTTTGCGTATTATTCAGTTTCAACGCGTTCG SEQ ID NO 2) primers were used to amplify the upstream fragment and the PL5 (5′CTGGAGCTCAGAGGAATAATACATGTCAAAAGTCG SEQ ID NO 3) and PL6 (5′ CTCGAGACAACAGCGATAATCACATCACC SEQ ID NO 4) primers were used to amplify the downstream fragment. A 0.3 kb DNA segment including the sequence of the Plac promoter was amplified by PCR using the pBluescript II KS plasmid (Addegen) as template and the following primers: PL3 (5′ GTTGAAACTGAATAATACGCAAACCGCCTCTC SEQ ID NO 5) and PL4 (5′ ATGTATTATTCCTCTGAGCTCCAGCTTTTGTTCC SEQ ID NO 6). The PL2 and PL3 primers have 5′ flanking sequences which make them complementary to each other. The PL4 and PL5 primers were designed in a similar manner. The three amplified DNA fragments were spliced by overlapping using the PL1 and PL6 primers. The thus obtained 2.1 kb DNA fragment was digested with the XhoI restriction enzyme and then cloned in the SalI restriction site of the pKO3 suicide vector. The chromosomal insertion of the Plac promoter was then carried out according to the protocol described in Link, A. J., Phillips, D., Church, G. M., 1997. Methods for generating precise deletions and insertions in the genome of wild-type Escherichia coli: application to open reading frame characterization. J. Bacteriol. 179, 6228-6237. [7]. The pKO3 plasmid was provided by the Church laboratory (Harvard University, USA). Positive clones were screened by PCR (The PCR cycle was 1 cycle of 3 minutes at 95° C., 35 cycles of 30 s at 95° C., 30 s at 53° C. and 1 minute at 68° C., 1 cycle 5 minutes at 68° C. The polymerase used was DNA Taq polymerase (NEB) for the presence of an amplified fragment of 1.396 kb DNA using the PL3 and PL7 primers 5′TCGAGGTCATTAGCCACCA SEQ ID NO 7).

### 1.2. Generation of Deletion Mutants

The selected ptsG, manXYZ and pfkA genes were inactivated using the pKO3 protocol as described in Link, A. J., Phillips, D., Church, G. M., 1997. Methods for generating precise deletions and insertions in the genome of wild-type Escherichia coli: application to open reading frame characterization. J. Bacteriol. 179, 6228-6237, 1997 [7]. To obtain the deletion mutant of the ptsG gene, a 351 bp DNA segment located between nucleotides 568 and 919 of ptsG was deleted and replaced by the 5′AGC sequence as follows: two DNA segments flanking the sequence to be deleted were amplified by PCR. The PCR cycle was 1 cycle of 30 s at 98° C., 35 cycles of 15 s at 98° C., 15 s at 70° C. and 30 s at 72° C., 1 cycle of 5 minutes at 72° C. The polymerase used was Phusion (NEB) using the genomic DNA of the E. coli K-12 strain as a template. The 900 bp upstream fragment was amplified with the (5′CTAGGATCCGCCGAAAATTGGGCGGTGAATAAC SEQ ID NO 8) and (5′GTAAAGCTTCTGGTAAGCAGCCCAGAGAAG SEQ ID NO 9) primers and the 958 bp downstream fragment was amplified with the (5′CTCAAGCTTGCGCCGATCCTGTACATCATCC SEQ ID NO 10) and (5′CGTGTCGACCGTTAATCCTAATCTGCCACGCACC SEQ ID NO 11) primers. The two amplified fragments were ligated at their terminal HindIII restriction site and cloned together at the BamHI and SalI restriction sites of the pKO3 suicide vector. The deletion was then carried out according to the protocol described in Link, A. J., Phillips, D., Church, G. M., 1997. Methods for generating precise deletions and insertions in the genome of wild-type Escherichia coli: application to open reading frame characterization. J. Bacteriol. 179, 6228-6237 [7].

In the genetic background/strain described above, a 1.219 kb fragment located between nucleotides 332 of manX and 522 of manY was deleted to obtain the deletion mutant of the manXYZ genes, as follows: two DNA fragments flanking the sequence to be deleted were amplified by PCR using the genomic DNA of the E. coli K-12 strain as a template. The 915 bp upstream fragment was amplified with the (5′ACTCGAGAATGGCGATGAAGAGAG SEQ ID NO 12) and (5′CCAGTGCCACCAGTTCATC SEQ ID NO 13) primers and the 902 bp downstream fragment was amplified with the (5′CAAGCTTCGATTCCGGAAGTGGTGAC SEQ ID NO 14) and (5′AGGATCCATGCCCCCATGACAAACAG SEQ ID NO 15) primers. The two amplified fragments were ligated at their terminal HindIII restriction site and cloned together at the BamHI and Sal restriction sites of the pKO3 suicide vector. The deletion was then carried out according to the protocol described in Link, A. J., Phillips, D., Church, G. M., 1997. Methods for generating precise deletions and insertions in the genome of wild-type Escherichia coli: application to open reading frame characterization. J. Bacteriol. 179, 6228-6237 [7]. The thus obtained strain was named “MGX”.

To obtain the deletion mutant of the pfkA gene in the genetic background of the MGX strain, a 396 bp DNA segment located between nucleotides 146 and 542 of the pfkA gene was deleted and replaced by the (5′AAGCTT) sequence as follows: two DNA segments flanking the sequence to be deleted were amplified by PCR using the genomic DNA of E. coli K-12 as template. The 890 bp upstream fragment was amplified with the (5′GGATCCAGGCGTCGGGGATATCGTG SEQ ID NO 16) and (5′AAGCTTCGGTCTTCATACAGACCCAGATAGC SEQ ID NO 17) primers and the 931 bp downstream fragment was amplified with the (5′AAGCTTGGCCATTGCCGGGGGCTGTG SEQ ID NO 18) and (5′CTCGAGCCACCGTGTGACTGACGAATC SEQ ID NO 19) primers. The two amplified fragments were ligated at their terminal HindIII restriction site and cloned at the BamHI and Sal restriction sites of the pKO3 suicide vector. The deletion was then carried out according to the protocol described in Link, A. J., Phillips, D., Church, G. M., 1997. Methods for generating precise deletions and insertions in the genome of wild-type Escherichia coli: application to open reading frame characterization. J. Bacteriol. 179, 6228-6237 [7]. The thus obtained strain is named “PFKA”.

### 1.3. Cloning the Galp Gene on a Plasmid (Primers and Plasmid Sequence)

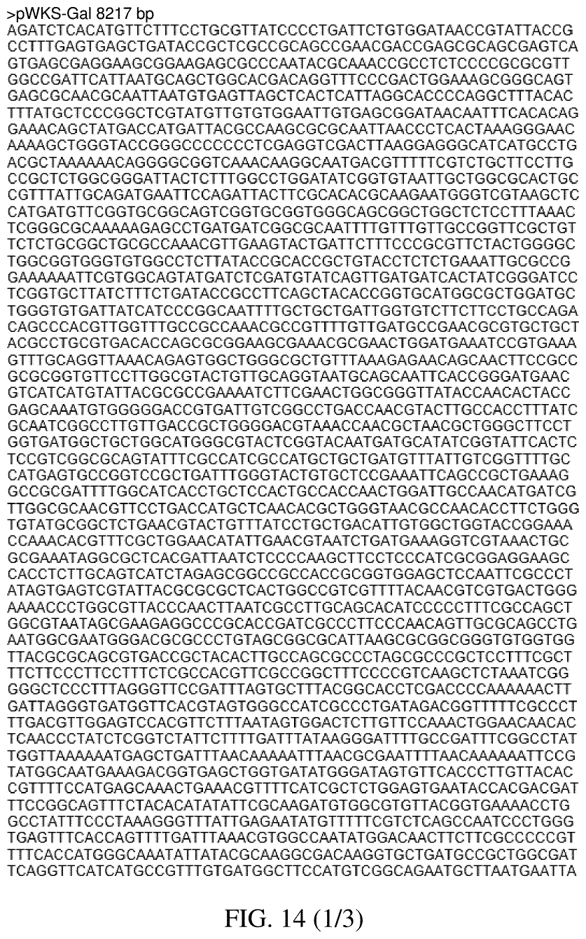

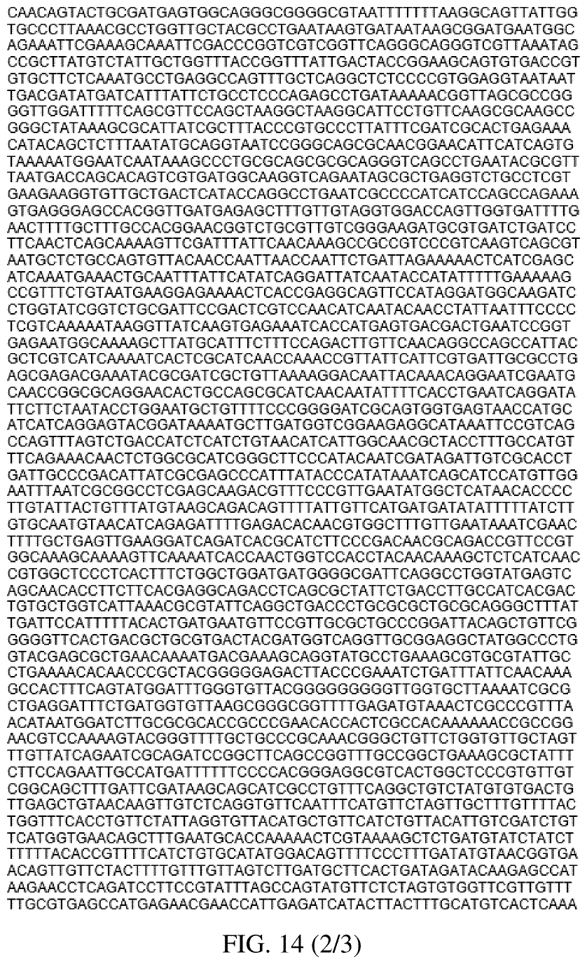

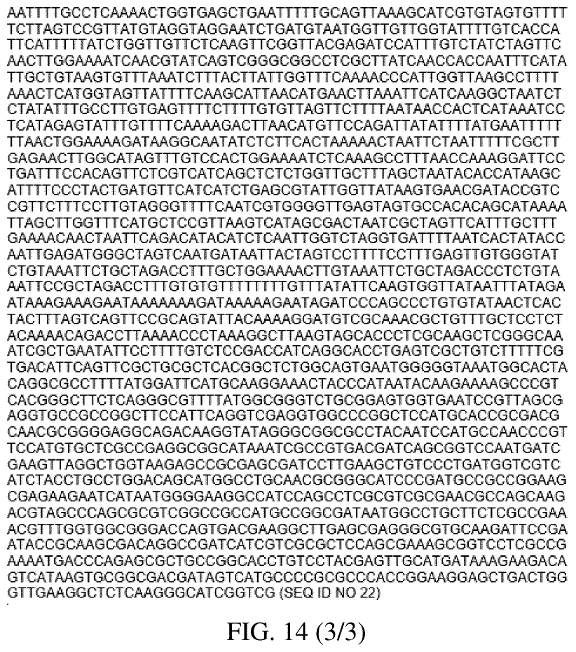

A 1.471 kb DNA fragment containing the sequence of the galP gene was amplified by PCR. The PCR cycle was 1 cycle of 30 s at 98° C., 35 cycles of 15 s at 98° C., 15 s at 68° C. and 30 s at 72° C., 1 cycle of 5 minutes at 72° C. The polymerase used was Phusion (NEB) using the genomic DNA of the E. coli K-12 strain as template with the following primers: (5′TTGTCGACTTAAGGAGGGCATCATGCCTGAC SEQ ID NO 20) and (5′GTTCTAGATGACTGCAAGAGGTGGCTTCC SEQ ID NO 21). The amplified fragment was then cloned at the Sal and Xbal restriction sites of the pWKS130 expression vector as described in Rong Fu Wang, Kushner, S. R., 1991. Construction of versatile low-copy-number vectors for cloning, sequencing and gene expression in Escherichia coli. Gene 100, 195-199 [10] to form the pWKS-GalP plasmid. The resulting plasmid sequence is represented in FIG. 14 and corresponds to the nucleic acid sequence SEQ ID NO 22. This plasmid was transformed in the MGX and PFKA strains to give the “MGX1” and “PFKA1” strains respectively.

Table 7 below groups together the different strains and their genotype.

TABLE 7
GENOTYPE OF THE GENERATED MUTANTS

| Chassis strain | Genotype |
|---|---|
| MDO | endA1 recA1 gyrA96 thi-1 glnV44 relA1 hsdR17 lacZ- |
| | wcaF::Plac nanKETA lacA melA wcaj mdoH |
| MGX1 | MDO + ptsG manXYZ |
| | pKWS-galP |
| PFKAl | MDO + ptsG manXYZ pfkA |
| | pKWS-galP |

### 1.4. Genetic Validation of the Deletion Mutant of the Genes Encoding the PTS Transporters and the PFKA Gene

A verification of the effectiveness of the deletions of the targeted genes was performed via PCR controls on the MDO, MGX and PFKA strains.

### Pts Transport System Gene Deletion Mutants

Mutants in the PTS transport system were constructed by deleting a 350 bp fragment of the ptsG gene and a 1200 bp fragment of the manXYZ operon as described above.

The PCRs were carried out using genomic DNA from each strain as template using the following primers for checking the ptsG gene fragment deletion,

ptsG_FW:
(5′CTTCTCTCAGTGGGCTGCTTACCAG 3′ SEQ ID NO 23),
ptsG_RV:
(5′CCGTTAATCCTAATCTGCCACGCACC 3′ SEQ ID NO 24).

The PCR cycle was 1 cycle of 3 minutes at 95° C., 35 cycles of 30 s at 95° C., 30 s at 55° C. and 1 minute at 68° C., 1 cycle of 5 minutes at 68° C. The Polymerase used was the DNA Taq polymerase (NEB). The expected size of the amplified fragments is 1300 bp for the wild-type (WT) strain and 950 bp for the MGX and PFKA strains if the genetic constructions are correct and comprise the deletion.

The following primers were used for checking the manXYZ operon fragment deletion:

| | manXYZ_FW |
|---|---|
| (SEQ ID NO 25) | |
| | 5′ TGTTAGGCGAGCAGGAAAACGTCG 3′ |
| | manXYZ_RV |
| (SEQ ID NO 26) | |
| | 5′ ACCAATCACACCCAGAGCAACCAG 3′. |

In this case, the expected size of the amplified fragments is 1600 bp for the wild-type (WT) strain and 450 bp for the MGX and PFKA strains if the genetic constructions are correct and comprise the deletion.

The PCR control is carried out by migration of the amplified fragment in a 1% agarose gel in a MUPID-ONE electrophoresis tank (Eurogentec, Liege, Belgium). 5 μL of PCR product are homogenized with 1 μL of 6×″ loading buffer (NEB, Ipswich, Mass.) then deposited on a gel for migration in a 1×TAE buffer (Sigma, Seelze, Germany). 5 μL of 1 kB size marker (NEB) are also deposited in another well. The migration is performed at 100 V for 30 minutes. The gel is then incubated for 10 minutes in a solution containing 0.01% of ethidium bromide (Euromedex, Souffelweyersheim, France). The detection of the amplified fragments is performed via Gel DOC EZ (Biorad, Berkeley, Calif.) according to the manufacturer's instructions.

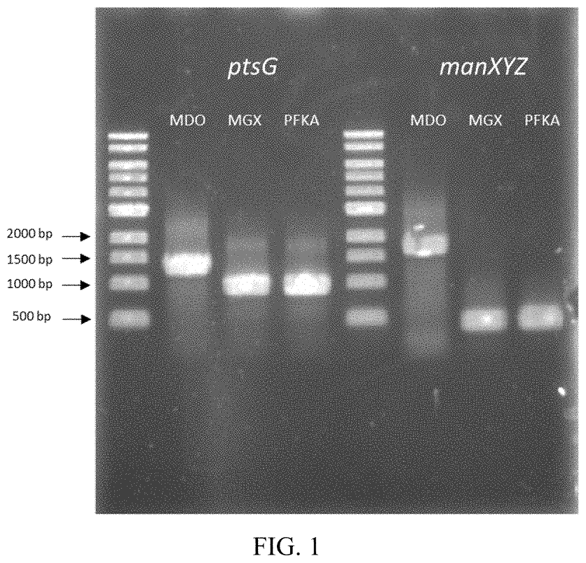

FIG. 1 is a picture showing the PCR products of DNA fragments amplified from the genome of the MDO, MGX and PFKA strains, and having migrated on an agarose gel.

As represented and demonstrated in FIG. 1, the size of the fragments amplified by PCR corresponds to that expected. Indeed, the amplified fragments from the MDO strain (without deletion) have a size of 1300 bp and 1600 bp for the ptsG gene and manXYZ operon fragments respectively, unlike those of the amplified fragments from the MGX and PFKA strains, which correspond to those expected, namely approximately 950 bp and 450 bp respectively, with the carried out deletions.

### PFKA Gene Deletion Mutants

The pfkA gene deletion mutant was constructed by deleting a 400 bp fragment of the pfkA gene as described above. The PCRs were carried out using genomic DNA from each strain as template using the following primers for checking the pfkA gene fragment deletion,

| | pfkA_FW: |
|---|---|
| (SEQ ID NO 27) | |
| | 5′ CATTTTGCATTCCAAAGTTCAGAGG 3′, |
| | pfkA_RV: |
| (SEQ ID NO 28) | |
| | 5′ TCATCGGTTTCAGGGTAAAGGAATCT 3′ |

The PCR cycle was 1 cycle of 3 minutes at 95° C., 35 cycles of 30 s at 95° C., 30 s at 55° C. and 1 s at 68° C., 1 cycle of 5 minutes at 68° C. The polymerase used was the DNA Taq polymerase (NEB). The expected size of the amplified fragments is 1000 bp for the MDO and MGX strains and 600 bp for the PFKA strain if the genetic constructions are correct and comprise the deletion.

The PCR control is carried out by migration of the ADN amplified fragment in a 1% agarose gel in a MUPID-ONE electrophoresis tank (Eurogentec, Liege, Belgium). 5 μL of PCR product are homogenized with 1 μL of 6×″ loading buffer (NEB, Ipswich, Mass.) then deposited on a gel for migration in a 1×TAE buffer (Sigma, Seelze, Germany). 5 μL of 1 kB size marker (NEB) are also deposited in another well. The migration is performed at 100 V for 30 minutes. The gel is then incubated for 10 minutes in a solution containing 0.01% of ethidium bromide (Euromedex, Souffelweyersheim, France). The detection of the amplified DNA fragments is performed via Gel DOC EZ (Biorad, Berkeley, Calif.) according to the manufacturer's instructions.

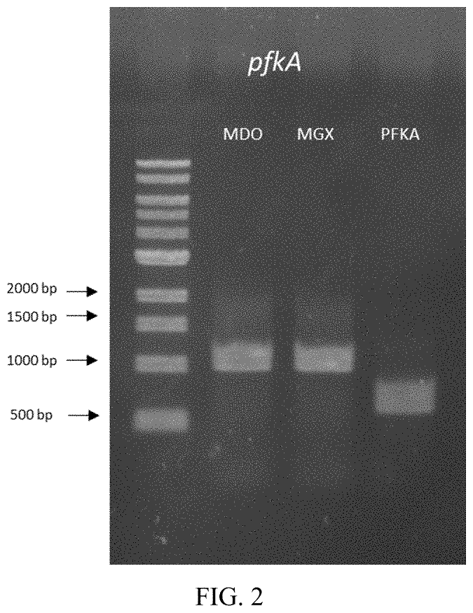

FIG. 2 is a picture showing the PCR products of DNA fragments amplified from the genome of MDO, MGX and PFKA strains, and having migrated on an agarose gel.

As represented and demonstrated in FIG. 2, the size of the fragment amplified from the genome of the PFKA strain was indeed less than that observed for the MDO and MGX strains, and corresponds to the expected size, namely around 600 bp, demonstrating that the deletion is present and correct.

### 1.5. Phenotypic Validation of the Deletion Mutant of Genes Encoding Pts Transporters and Overexpressing the Galp Gene

It is known that the deletion of the ptsG and manXYZ genes decreases the capacity of glucose transport in the cell of E. Coli. (Quanfeng Liang, 1 Fengyu Zhang, 1 Yikui Li, 1 Xu Zhang, 1 Jiaojiao Li, 1 Peng Yang, 1 and Qingsheng Qia, 1 Comparison of individual component deletions in a glucose-specific phosphotransferase system revealed their different applications Sci Rep. 2015; 5: 13200. Published online 2015 Aug. 19. [28], Sonja Steinsiek and Katja Bettenbrock Glucose Transport in Escherichia coli Mutant Strains with Defects in Sugar Transport Systems, J Bacteriol. 2012 November; 194(21): 5897-5908. [29]). To check that the deletion mutants of the genes encoding proteins of the transport systems known as “PTS” (PhosphoTransferase System)—strain named MGX—has the expected phenotype, the MDO and MGX strains were cultured in a minimum M9 medium (minimum M9 medium—M9 salts: Na2HPO₄ 12H₂O 17.4 g/L, KH₂PO₄ 3.02 g/L, NaCl 0.51 g/L, NH₄Cl 2.04 g/L. Trace metals and salts: Na2EDTA 2 H₂O 15 mg/L, ZnSO4 7 H₂O 4.5 mg/L, CoCl₂ 6H₂O 0.3 mg/L, MnCl2 4H₂O 1 mg/L, H₃BO₃ 1 mg/L, Na2MoO₄ 2 H₂O 0.4 mg/L, FeSO4 7 H₂O 3 mg/L, CuSO4 5 H₂O 0.3 mg/L. MgSO₄ 0.5 g/L CaCl₂) 4.38 mg/L, Thiamine hypochloride 0.1 g/L) using glucose as unique carbon source. In addition, to check that the overexpression of the galP gene allows to restore all or part of the glucose transport capacity and therefore the growth rate, the pWKS-GalP plasmid was transformed in the MGX strain. The resulting strain selected on LB+Kanamycin medium was named MGX1 (see above). This MGX1 strain was also cultured in the M9 medium with glucose.

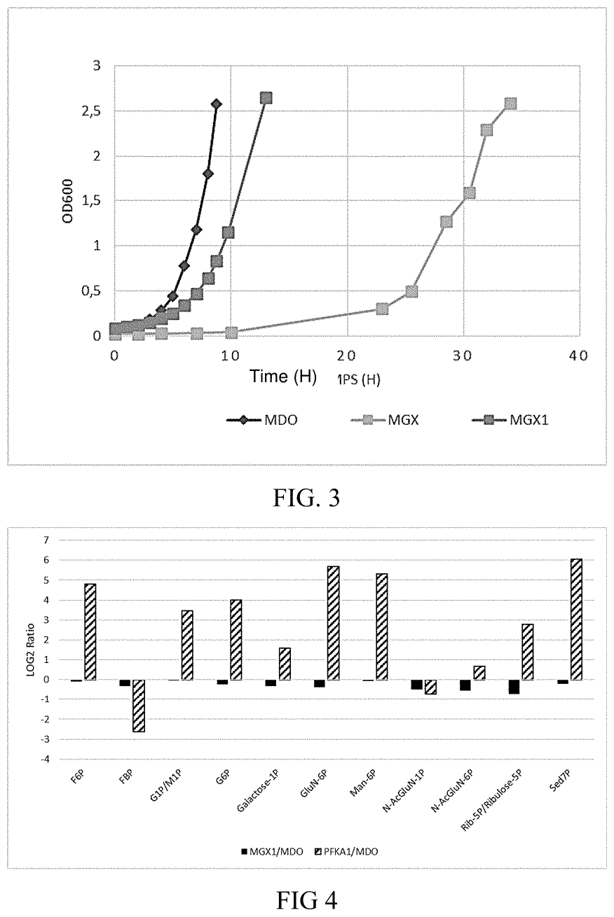

One hundred microliters of culture overnight (10 to 12 hours of culture in LB medium (Tryptone 10 g/L, Yeast extract 5 g/L, NaCl, 10 g/L) of the E. coli MDO, MGX and MGX1 strains were transferred into 250 mL baffled Erlenmeyer flasks containing 50 mL of minimum M9 medium supplemented with D-glucose (3 g/L) as unique carbon source. The thus inoculated Erlenmeyer flasks were incubated for 16 h at 37° C. with an orbital stirring of 220 revolutions per minute. The cells were then collected by centrifugation (Sigma, Seelze, Germany) for 10 min at 2 000 g at room temperature, i.e. 25° C., washed with sterile M9 medium and used to inoculate the 250 ml baffled Erlenmeyer flasks containing 50 mL of M9 medium supplemented with D-glucose (3 g/L) at OD_{600 nm}˜0,1. To trigger the expression of the galP gene carried by the pWKS-GalP plasmid, IPTG was added to the MGX1 strain culture medium at a final concentration of 60 μM. The cultures of the strains were carried out at 37° C. with an orbital stirring of 220 revolutions per minute (220 revolutions per minute). The growth was followed by measuring turbidimetry at an optical density of 600 nm using a Genesys 6 spectrophotometer (Thermo, USA). The results are shown in FIG. 3.

As represented in FIG. 3, unlike the growth of the MDO strain, that of the MGX strain is characterized by a long latency phase (10 h at least) which is a phenotypic feature reported in the literature, of this strain type (Liang, Q., Zhang, F., Li, Y., Zhang, X., Li, J., Yang, P., Qi, Q., 2015. Comparison of individual component deletions in a glucose-specific phosphotransferase system revealed their different applications. Sci. Rep. 5, 13200. [6]). The maximum growth rate (p) of this strain is 0.25 h⁻¹ (calculated between t=23 h and t=34 h), namely approximately half the growth rate of the MDO strain. The expression induction of the galP gene, in part, restores the growth rate. Indeed, the growth profile of the MGX1 strain is very similar to that of the MDO strain, with absence of latency phase and a growth rate (μ=0.32 h⁻¹) reaching 70% of that of the MDO strain (μ=0.45 h⁻¹). The results therefore clearly demonstrate that the MGX1 strain possess superior transport capacities with respect to the MGX strain.

### 1.6. Phenotypic Validation of the PTS-PFKA Deletion Mutant

### A. GROWTH CAPACITY

The growth capacity of the MDO, MGX, MGX1, PFKA1 strains was determined by independent culture of said strains, cultured in the M9 medium supplemented with glucose.

One hundred microliters of culture overnight (10 to 16 hours of culture in LB medium (Tryptone 10 g/L, Yeast extract 5 g/L, NaCl, 10 g/L) of the E. coli MDO, MGX and MGX1, PFKA1 strains were transferred into 250 mL baffled Erlenmeyer flasks containing 50 mL of M9 medium supplemented with D-glucose (3 g/L) as unique carbon source. The Erlenmeyer thus inoculated flasks were incubated for 16 h at 37° C. with an orbital stirring of 220 revolutions per minute (220 rpm). The cells were then collected by centrifugation (Sigma, Seelze, Germany) for 10 min at 2 000 g at room temperature, i.e. 25° C., washed with sterile M9 medium and used to inoculate the 250 ml baffled Erlenmeyer flasks containing 50 mL of M9 medium supplemented with D-glucose (3 g/L) at OD600 nm ˜0.1. To trigger the expression of the galP gene carried by the pWKS-GalP plasmid, IPTG was added to the culture medium of the MGX1 and PFKA1 strains at a final concentration of 60 μM. The cultures of the strains were carried out at 37° C. with an orbital stirring of 220 revolutions per minute (220 rpm). The growth was followed by measuring turbidimetry at an optical density of 600 nm using a Genesys 6 spectrophotometer (Thermo, USA).

The growth capacity of the strains was determined by measuring the growth rate as described in Growth Rates Made Easy Barry G. Hall, Hande Acar, Anna Nandipati, Miriam Barlow Author Notes Molecular Biology and Evolution, Volume 31, Issue 1, January 2014, Pages 232-238, https://doi.org/10.1093/molbev/mst187 28 Oct. 2013 of the different strains.

The obtained results are represented in Table 8 below.

TABLE 8
GROWTH RATE OF THE DIFFERENT STRAINS
CULTURED IN GLUCOSE M9 MEDIUM (3 G/L)

| Chassis strain | Growth rate in glucose M9 medium (h⁻¹) |
|---|---|
| MDO | 0.45 |
| MGX | 0.18 |
| MGX1 | 0.32 |
| PFKA1 | 0.15 |

As indicated above, the pfkA gene, encoding the phosphofructokinase, was deleted from the genome of the E. coli MGX strain, generating the E. coli PFKA strain (see above). The obtained results clearly show that the absence of pfkA caused a decrease in the growth rate in M9 medium with glucose (3 g/L), and this strain is no longer capable of growing in M9 medium supplemented with glucose (data not shown). The introduction of the GalP gene in this genetic background (E. coli PFKA strain) made it possible to obtain the designated E. coli PFKA1 strain. The measured growth rate of the E. coli PFKA1 strain was close to that of the MGX strain showing a “restoration” of the strain growth capacity despite the absence of the pfkA gene (0.15 h⁻¹ or one third of the growth rate of the MDO strain).

### B. Metabolomic Analysis

Another characteristic phenotypic feature of the pfkA gene deletion mutant is the intracellular accumulation of Glucose-6-Phosphate (G6P) and Fructose-6-Phosphate (F6P) (Ishii, N., Nakahigashi, K., Baba, T., Robert, M., Soga, T., Kanai, A., Hirasawa, T., Naba, M., Hirai, K., Hoque, A., Ho, P. Y., Kakazu, Y., Sugawara, K., Igarashi, S., Harada, S., Masuda, T., Sugiyama, N., Togashi, T., Hasegawa, M., Takai, Y., Yugi, K., Arakawa, K., Iwata, N., Toya, Y., Nakayama, Y., Nishioka, T., Shimizu, K., Mori, H., Tomita, M., 2007. Multiple High-Throughput Analyses Monitor the Response of E. coli to Perturbations. Science 316, 593-597. [4]). Checking the accumulation of Glucose-1-Phosphate (G1P) and Mannose-1-Phosphate (M1P), necessary phosphorylated monosaccharides for the synthesis of oligosaccharides by glycoside-phosphorylases (GP), in the PFKA1 strain was carried out by quantitative analysis of the metabolome (intracellular metabolites). In particular, the determination of the concentration of hexose phosphates, in particular of Glucose-1-Phosphate (G1P) and Mannose-1-Phosphate (M1P) was carried out as follows.

### B.1. Sampling and Extraction of Intracellular Metabolites

One hundred microliters of a culture in LB (lysogeny broth) medium (10 g/l of NaCl), overnight (i.e. 12 to 16 hours), of the different strains were used to inoculate 50 ml baffled Erlenmeyer flasks containing 10 mL of M9 medium supplemented with glucose at a final concentration of 3 g/L. The Erlenmeyer flasks were incubated for 24 h at 37° C. with an orbital stirring of 220 revolutions per minute (rpm). The cells were then collected by centrifugation (Sigma, Seelze, Germany) for 10 min at 2 000 g and at room temperature, (25° C.), then washed with M9 medium diluted five times, and used to inoculate, at an optical density of 600 nm of OD_{600 nm}=0.1, 50 mL baffled Erlenmeyer flasks containing 50 mL of diluted M9 medium supplemented with 3 g/L of glucose and incubated at 37° C. with an orbital stirring of 220 revolutions per minute (rpm). The cellular growth was followed by measuring turbidimetry at an optical density of 600 nm using a Genesys 6 spectrophotometer (Thermo, USA). In the exponential growth phase, 120 μL of the culture was sampled and immediately mixed with 1.25 mL of a solution of acetonitrile/methanol/H₂O (4:4:2) at −20° C. to simultaneously block the metabolic activity and extract metabolites; 60 μL of a cell extract containing completely ¹³C-labeled metabolites were added to each sample to be used as internal standards as described in Mashego, M. R., Wu, L., Van Dam, J. C., Ras, C., Vinke, J. L., Van Winden, W. A., Van Gulik, W. M., Heijnen, J. J., 2004. MIRACLE: mass isotopomer ratio analysis of U⁻¹³C-labeled extracts. A new method for accurate quantification of changes in concentrations of intracellular metabolites. Biotechnol. Bioeng. 85, 620-628. [9] and Wu, L., Mashego, M. R., van Dam, J. C., Proell, A. M., Vinke, J. L., Ras, C., van Winden, W. A., van Gulik, W. M., Heijnen, J. J., 2005. Quantitative analysis of the microbial metabolome by isotope dilution mass spectrometry using uniformly 13C-labeled cell extracts as internal standards. Anal. Biochem. 336, 164-171. [12]

The samples were placed at −20° C. for 20 minutes, then centrifuged for 10 minutes at 10,000 g at 4° C. to remove cell debris. The supernatant was then dried under vacuum using a vacuum concentrator (SC110A SpeedVac Plus, ThermoSavant, Waltham, Mass., USA) and stored at −80° C. until analysis.

### B.2. IC-ESI-HRMS Analysis of Metabolites

For the analysis, the samples were taken up in 120 μL of ultrapure water, then analyzed by ion chromatography (Thermo Scientific Dionex ICS-5000+ system, Dionex, Sunnyvale, Calif., USA) coupled to a mass spectrometer (LTQ Orbitrap mass spectrometer, Thermo Fisher Scientific, Waltham, Mass., USA) equipped with an electrospray type ionization source. The ion chromatography method is a variant of that described by Kiefer et al., 2007 (Kiefer, P., Nicolas, C., Letisse, F., Portais, J.-C., 2007. Determination of carbon labeling distribution of intracellular metabolites from single fragment ions by ion chromatography tandem mass spectrometry. Anal. Biochem. 360, 182-188. [5]). The KOH gradient was changed as follows: 0 min, 0.5 mM; 1 min, 0.5 mM; 9.5 min, 4.1 mM; 12.5 min, 30 mM; 24 min, 50 mM; 36 min, 60 mM; 36.1 min, 90 mM; 43 min, 90 mM; 43.5 min, 0.5 mM; 48 min, 0.5 mM. All gradients are linear. KOH gradient related signal deletion was performed by an electrochemical anionic suppressor (AERS 300-2 mm, Dionex, Sunnyvale, Calif., USA). The applied electrolysis current was 87 mA, in external regeneration mode using ultrapure water. The sample injection volume was 35 μL. Mass spectrometry analysis was carried out in negative mode, at a resolution of 30,000 in “fullscan” mode, with the following source parameters: source capillary temperature, 350° C.; source temperature, 300° C.; “sheath gas” flow rate, 50 a.u. (arbitrary unit); auxiliary gas, 5 a.u; S-Lens radio frequency (RF) level, 60%; and ionization spray voltage, 3.5 kV. Data were acquired with the Xcalibure software and processed with the TraceFinder 3.1 software (Thermo Fisher Scientific, Waltham, Mass., USA). Using calibration curves, the intracellular concentrations of each metabolite for the different strains were determined. FIG. 4 shows the concentration ratios of each metabolite between the MGX1 and PFKA1 strains and the MDO strain, in order to better visualize the differences. The ratios were calculated in Log₂. As represented in FIG. 4, the concentrations of the metabolites in the MGX1 strain and those in the MDO strain are very similar and no significant difference was highlighted. The deletion of the PTS transport system therefore has no major effect on this part of the metabolic network.

As demonstrated in FIG. 4, the metabolite accumulation profile in the PFKA1 strain is different from that of the MDO strain. For example, the intracellular concentration of Fructose-1,6-Bisphosphate (FBP) is lower than that measured in the MDO strain. The absence of phosphofructokinase advantageously allows an increase in intracellular concentrations of F6P and G6P (Ishii, N., Nakahigashi, K., Baba, T., Robert, M., Soga, T., Kanai, A., Hirasawa, T., Naba, M., Hirai, K., Hoque, A., Ho, P. Y., Kakazu, Y., Sugawara, K., Igarashi, S., Harada, S., Masuda, T., Sugiyama, N., Togashi, T., Hasegawa, M., Takai, Y., Yugi, K., Arakawa, K., Iwata, N., Toya, Y., Nakayama, Y., Nishioka, T., Shimizu, K., Mori, H., Tomita, M., 2007. Multiple High-Throughput Analyses Monitor the Response of E. coli to Perturbations. Science 316, 593-597. [4]). As represented and demonstrated in FIG. 4, the intracellular concentration of other phosphorylated monosaccharides is advantageously also higher in the PFKA1 strain. For example, the concentrations of Mannose-6-Phosphate (M6P), Galactose-1-Phosphate (Gal1P) and the total concentration of Glucose-1-Phosphate (G1P) and Mannose-1-Phosphate (M1P)—these two phosphorylated monosaccharides, which cannot be separated either by ion chromatography or by mass spectrometry, are significantly increased in the PFKA1 strain. These results therefore clearly demonstrate that the strain according to the invention advantageously allows to accumulate the necessary phosphorylated monosaccharides for the synthesis of oligosaccharides. These results also clearly demonstrate that the strain according to the invention advantageously allows, especially due to the accumulation of the necessary phosphorylated monosaccharides for the synthesis of oligosaccharides, to be an advantageous support and/or means for the production of oligosaccharides.

In addition, the results clearly demonstrate that the strains according to the invention allow the synthesis of oligosaccharides and advantageously their excretion in the culture medium. Thus, the production, recovery and isolation of oligosaccharides do not require any alteration or destruction of the strains, advantageously allowing a continuous production.

### Example 2: Production of Oligosaccharides with the Strain Filed with the CNCM (Collection Nationale De Culture De Microorganismes, Institut Pasteur, 25 Rue Du Docteur Roux, 75724 Paris Cedex 15, France), Under Number CNCM I-5499

In this example the strain filed with the CNCM under number CNCM I-5499, also named PFKA1 strain or E. Coli PFKA1 strain in Example 1 above was used for the production of oligosaccharides. To do this, the strain was transformed with expression vectors as follows.

1. Transformation of the PFKA1 Strain with the Plasmids Carrying the Genes Encoding a GP

### 1.1. Cloning of the Gene Encoding the Teth514-1788 Enzyme in the Pbad-Hisa and Ptrc Plasmids (Primers and Plasmid Sequence)

The gene encoding the Teth514-1788 protein of the Thermoanaerobacter sp. X-514 strain (Teth514-1788, Genbank accession number ABY93073.1) belonging to the GH130 family of the CAZy classification (http://www.cazy.org/) (Lombard, V., Golaconda Ramulu, H., Drula, E., Coutinho, P. M., Henrissat, B., 2014. The carbohydrate-active enzymes database (CAZy) in 2013. Nucleic Acids Res. 42, D490-D495 [30]) was synthesized and cloned in the pBAD HisA vector by the Biomatik company (Biomatik, Ontario, Canada) between the NcoI and XhoI restriction sites.

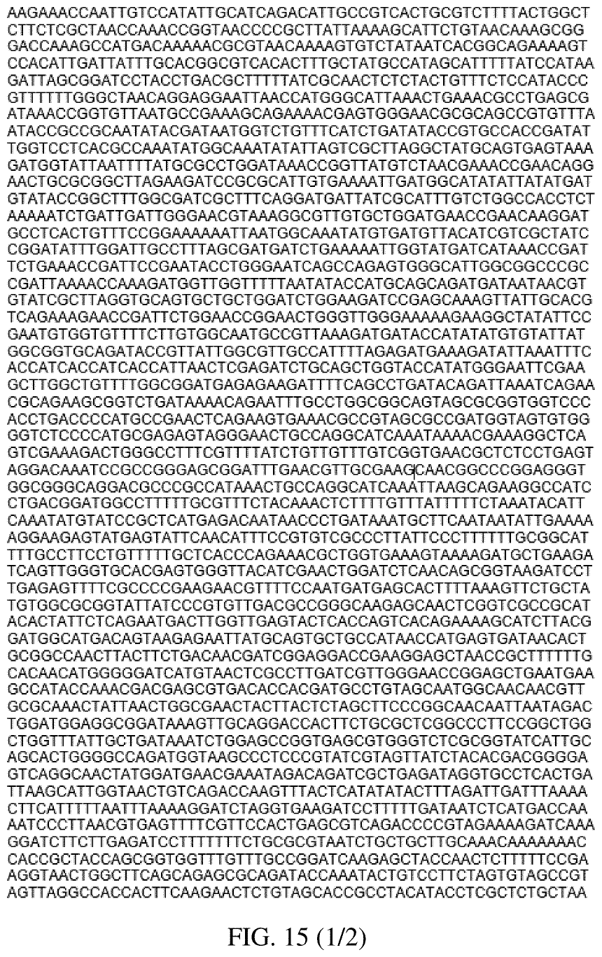

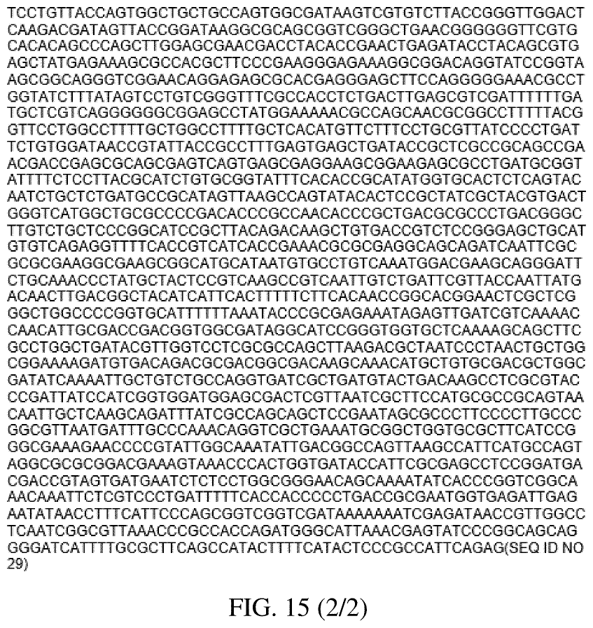

The obtained plasmid sequence corresponds to sequence SED ID NO 29 represented in FIG. 15. The transformed strain was cultured in M9 medium supplemented with mannose (20 g·L⁻¹) at 37° C. with stirring 180 revolutions per minute (180 rpm) for 6 days. The addition of 0.1% L-arabinose in the culture medium when the OD₆₀₀ is ≈0.4 allows the expression of the gene present in the plasmid and the production of a Teth514-1788-(His)₆ fusion protein, of 36.7 kDa.

The gene encoding the Teth514-1788 protein was also cloned in the pTRC-His-A vector between the BamH1 and EcoR1 restriction sites. The pTRC-His-A vector and the insert (coding sequence of the Teth514-1788 enzyme) were digested with the BamH1 and EcoR1 restriction enzymes according to the supplier's protocol (https://international.neb.com/protocols/2014/05/07/double-digest-protocol-with-standard-restriction-enzymes [47]). Beforehand, the BamH1 and EcoR1 restriction sites were added to the ends of the insert by polymerase chain reaction (PCR) using the primers fw: 5′-GAGGAAGGATCCATGGGCATTAAACTG-3′ (SEQ ID NO 31) and rev: 5′-GCTGCAGATGAATTCTTAATGGTG-3′ (SEQ ID NO 32) according to the supplier's protocol: https://international.neb.com/protocols/0001/01/01/pcr-protocol-m0530 [48]. The ligation of the insert and the vector purified on agarose gel is carried out by the T4 DNA ligase according to the supplier's protocol (https://international.neb.com/protocols/0001/01/01/dna-ligation-with-t4-dna-ligase-m0202 [49]).

The transformed strain was cultured in a M9 medium supplemented with mannose (20 g·L⁻¹) at 37° C. with stirring at 180 rpm for 6 days. The addition of 1 mM isopropyl-β-D-thiogalactopyranoside (IPTG) in the culture medium when the OD₆₀₀ is ˜ 0.4 allows the expression of the gene which leads to the synthesis of a (His)₆-Teth514-1788 recombinant protein, of 38.9 kDa.

The E. coli PFKA1 strain was transformed either with the pBAD-His-Teth514-1788 plasmid or with the pTRC-His-Teth514-1788 plasmid.

The transformation process was identical regardless of the plasmid and corresponded to the standard protocol for transforming chemocompetent cells. (https://www.addgene.org/protocols/bacterial-transformation/[50])

The Teth514-1788-(His)6 protein is also named herein Teth-1788.

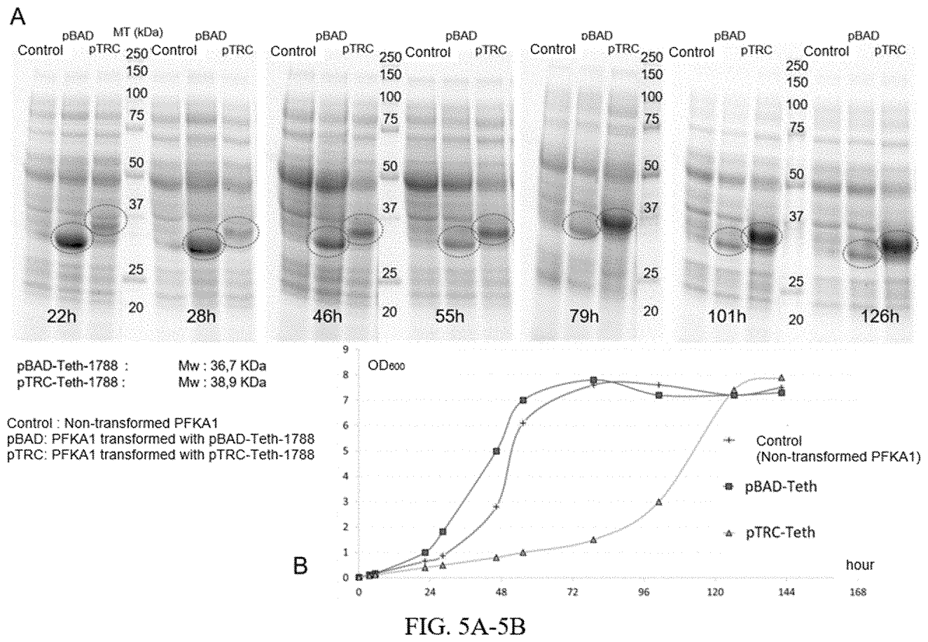

The transformed strains were cultured in Erlenmeyer flask in a M9 medium supplemented with mannose (FIG. 5). The conditions for carrying out these cultures were identical to those in Example 1 above.

Protein expression profiles were determined by SDS-PAGE Any KD electrophoresis gel from Bio-Rad. The cell pellets are lysed in 5 mM Tris HCl buffer containing lysosyme (Euromedex ref 5934-C) at 0.5 mg·mL-1 and DNAse (NEB, ref M0303L) at 50 μL (100 U) in 100 mL of 5 mM Tris HCl buffer. All the pellets were taken up in a buffer volume to be at an OD600 of 45 and incubated for 30 min at 37° C., then frozen at −20° C. The samples were thawed, centrifuged 25 min at 5000 g. 15 μL of each supernatant was taken up with 5 μL of loading dye blue (NEB, ref 50994905) were incubated 10 min at 95° C. before being placed on gel for a migration of 35 min at 100V in an electrophoresis tank in order to check if the Teth-1788 protein was indeed produced in the PFKA1 strain transformed with the pBAD-His-Teth514-1788 or pTRC-His-Teth514-1788 vectors.

As demonstrated in FIG. 5A, the obtained results demonstrate a production of Teth-1788 proteins following induction regardless of the expression vector used. In fact, as demonstrated in FIG. 5 A, after addition to the medium of the inducer, namely arabinose or IPTG, the intensity of the bands corresponding to the molecular weight of the Teth-1788 proteins increases, with a difference concerning the enzyme intracellular production kinetics.

This example clearly demonstrates that the E. coli PFKA1 strain can be i) transformed with expression vectors; and/or ii) used for the expression of genes encoding recombinant proteins

### 1.2 Cloning of the Gene Encoding the Uhgb_Mp Enzyme in the Pbad-Hisa and Ptrca Plasmids (Primers and Plasmid Sequence)

The gene encoding the “Unknown human gut bacteria_Mannoside-Phosphorylase” protein, a β-1,4-mannopyranosyl-chitobiose phosphorylase belonging to the GH130 family (Uhgb_MP, Genbank accession number ADD61463.1) was cloned in the pBAD HisA vector with primers:

| | Uhgb fw: |
|---|---|
| (SEQ ID NO 33) | |
| | 5′-CACCATGAGTATGAGTAGCAAAGTTATT-3′ |
| | and |
| | Uhgb rev: |
| (SEQ ID NO 34) | |
| | 5′-GATGATGCTTGTACGTTTGGTAAATTC-3′. |

The cloning process was that described in the document http://tools.thermofisher.com/content/sfs/manuals/pentr_dtopo_man.pdf [51].

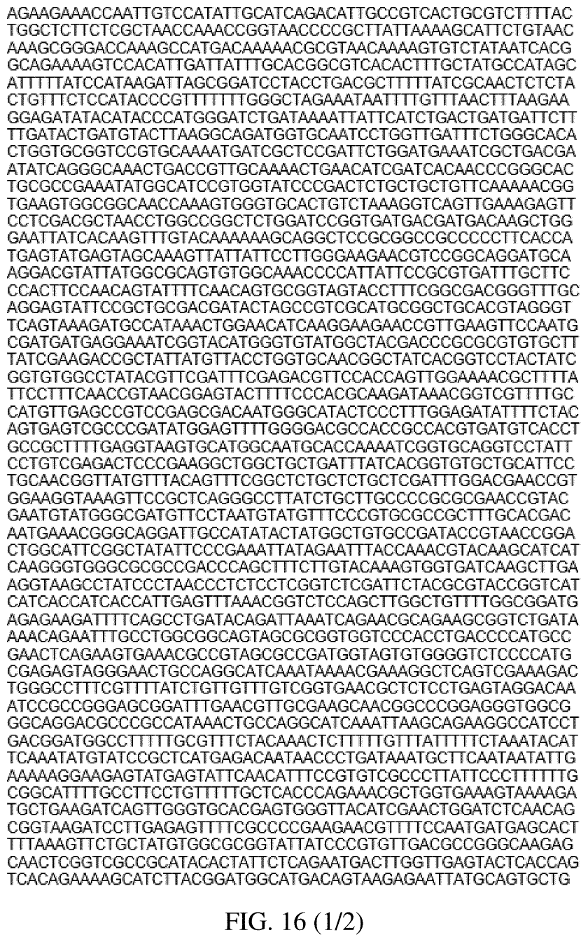

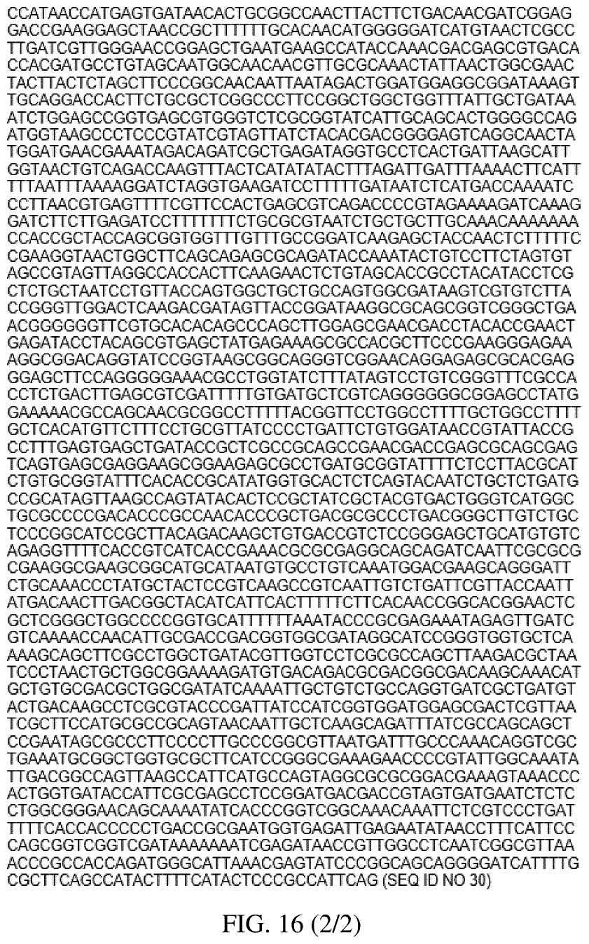

The obtained plasmid sequence corresponds to sequence SED ID NO 30 represented in FIG. 16. The transformed strain was cultured in a M9 medium supplemented with mannose (20 g·L⁻¹) at 37° C. with stirring at 180 rpm for 6 days. The addition of 0.1% L-arabinose in the culture medium when the OD₆₀₀ is ≈0.4 allows the expression of the gene present in the plasmid and the production of a Uhgb_MP-(His)₆ recombining protein, of 56.3 kDa (fused with a thioredoxin).

The gene encoding the Uhgb_MP-(His)₆ fusion protein was also cloned in the pTRC-His-A vector between the BamH1 and EcoR1 restriction sites. The transformed strain was placed in culture in a M9 medium supplemented with mannose (20 g·L⁻¹) at 37° C. with stirring (180 revolutions per minute (rpm), Infors HT Multitron) for 6 days. The addition of 1 mM isopropyl-β-D-thiogalactopyranoside (IPTG) in the culture medium when the OD₆₀₀ is ≈0.4 for the expression of the gene by adding isopropyl-β-D-thiogalactopyranoside (IPTG) which lead to the synthesis of a (His)₆-Uhgb_MP fusion protein, of 43.6 kDa.

The E. coli PFKA1 strain was transformed either with the pBAD-His-Uhgb_MP plasmid or with the pTRC-His-Uhgb_MP plasmid.

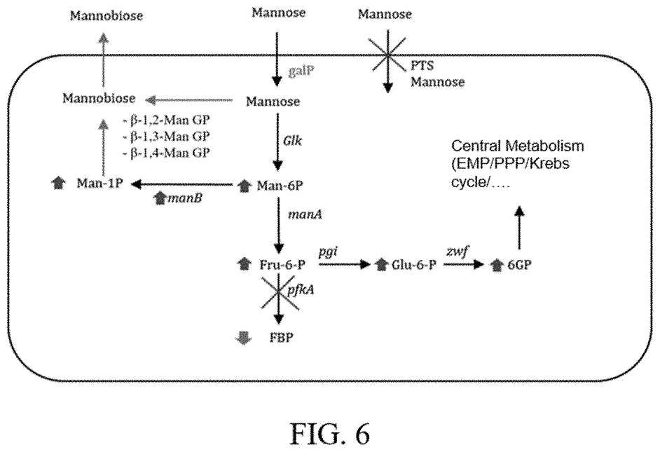

The scheme of FIG. 6 represents the central metabolism of the transformed E. coli PFKA1 strain indicating the modifications of the genetically modified metabolic pathways, the connection with the synthetic reactions of β-1,2-mannobiose and β-1,4-mannobiose by the respective glycoside phosphorylases (GPs) and the effects on the intracellular accumulation of substrates of these enzymes.

### 1.3 Cloning of the Gene Encoding a β-1,3-Mannooligosaccharide Phosphorylase Enzyme in the PBAD-HISA Plasmid (Primers and Plasmid Sequence)

The synthetic gene encoding the protein of a β-1,3-mannooligosaccharide phosphorylase was provided by the Biomatik Limited company (Cambridge, Ontario, Canada) with an optimization of the use of codons for gene expression in E. coli. This enzyme belongs to the GH130 family (accession number GigaBase 340101.Vvad_PD3074). This gene originally in the pET23a(+) plasmid was cloned in the pBAD HisA plasmid by the “In-Fusion cloning” method with primers: fw: 5′—GAGGAATTAACCATGCTGAGCGTGGAAAAACGCTG-3′ (SEQ ID NO 35) and rev: 5′-AGCTGCAGATCTCGATCAGTGGTGGTGGTGGTGGTGCTCGA−3′ (SEQ ID NO 36).

The cloning process was that described on the cloning kit supplier site: https://www.takarabio.com/learning-centers/cloning/in-fusion-cloning-overview [58].

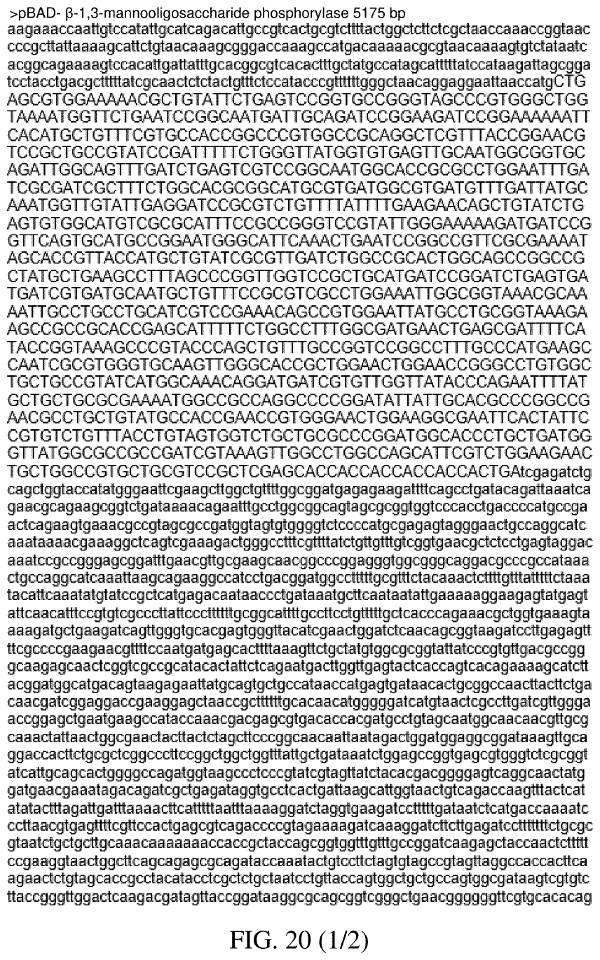

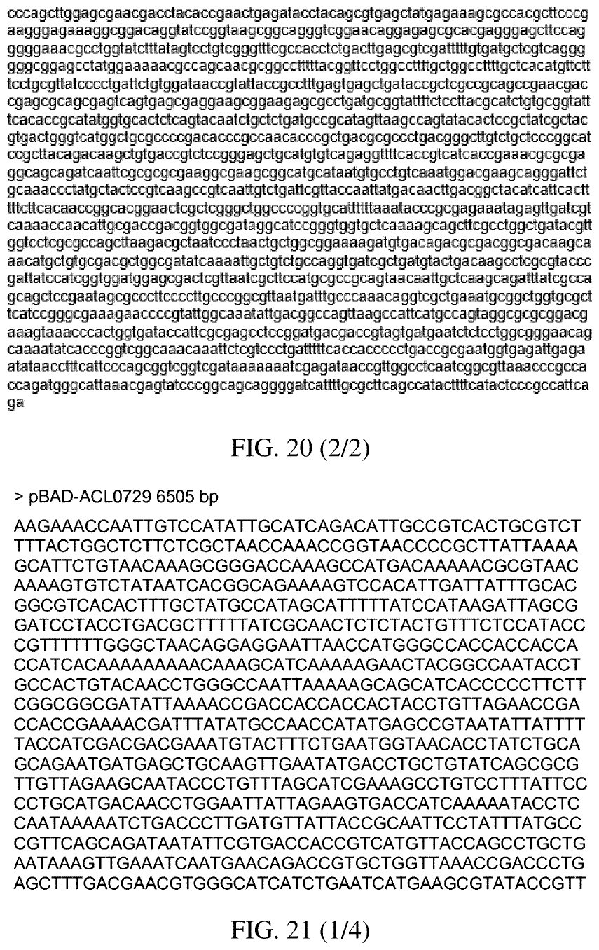

The obtained plasmid sequence corresponds to sequence SED ID NO 37 represented in FIG. 20. The transformed strain was cultured in a M9 medium supplemented with mannose (20 g·L⁻¹) at 37° C. with stirring at 180 rpm for 6 days. The addition of 0.1% L-arabinose in the culture medium when the OD₆₀₀ is ≈0.4 allowed the expression of the gene present in the plasmid and the production of the (His)₆-β-1,3-mannooligosaccharide phosphorylase recombining protein, of molar mass 44.4 kDa.

The transformed strain was cultured in a M9 medium supplemented with mannose (20 g·L⁻¹) at 37° C. with stirring at 180 rpm for 6 days. The E. coli PFKA1 strain was transformed with the pBAD-β-1,3-mannooligosaccharide phosphorylase plasmid.

The scheme of FIG. 6 represents the central metabolism of the transformed E. coli PFKA1 strain indicating the modifications of the genetically modified metabolic pathways, the connection with the synthesis reactions of β-1,2-mannobiose, β-1,3-mannobiose and β-1,4-mannobiose by the respective glycoside phosphorylases (GPs) and the effects on the intracellular accumulation of substrates of these enzymes.

### 1.4 Cloning of the Gene Encoding a Laminaribiose Phosphorylase Enzyme in the PBAD-HISA Plasmid (Primers and Plasmid Sequence)

The gene encoding the ACL0729 protein of the Acholeplasma laidlawii PG-8A strain (ACL0729, Genbank accession number ABX81345.1) belonging to the GH94 family of the CAZy classification (http://www.cazy.org/) (Lombard, V., Golaconda Ramulu, H., Drula, E., Coutinho, P. M., Henrissat, B., 2014. The carbohydrate-active enzymes database (CAZy) in 2013. Nucleic Acids Res. 42, D490-D495 [30]) was synthesized and cloned in the pBAD HisA vector by the GeneCust company (GeneCust, Boynes, France) between the NcoI and XhoI restriction sites.

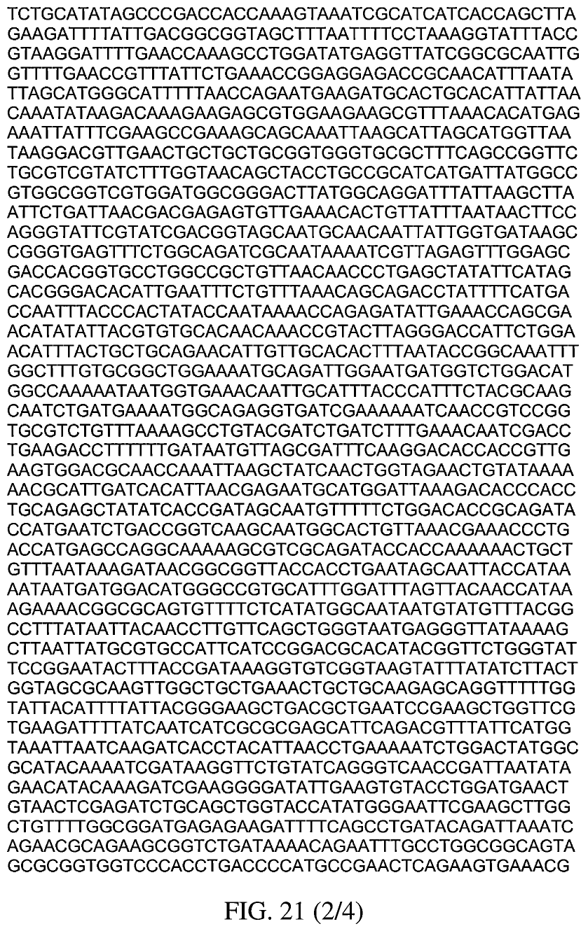

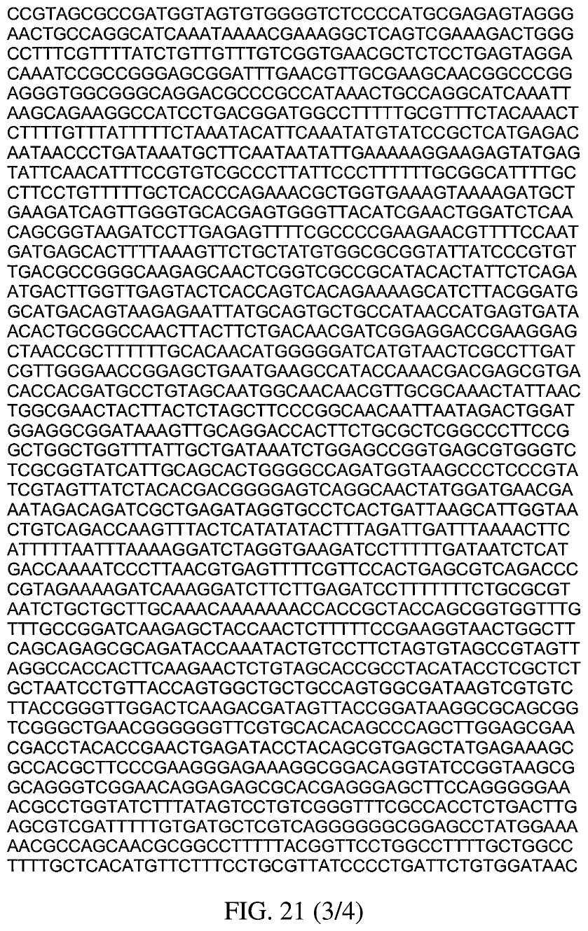

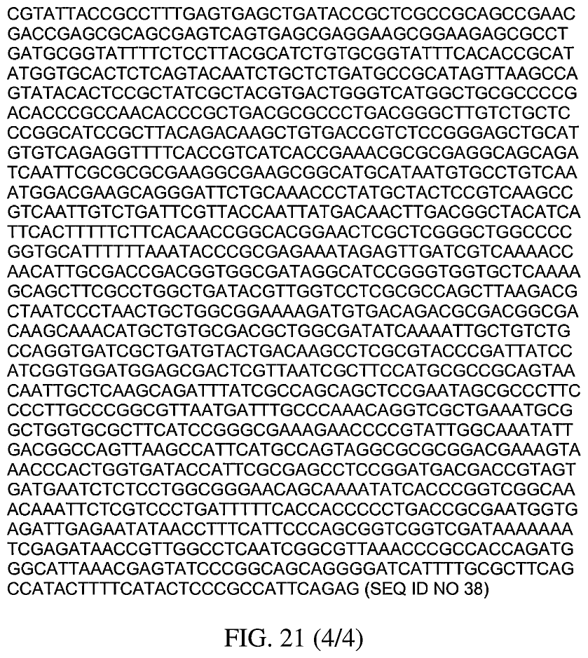

The obtained plasmid sequence corresponds to sequence SED ID NO 38 represented in FIG. 21. The transformed strain was cultured in M9 medium supplemented with glucose (15 g·L-¹) at 37° C. with stirring 180 revolutions per minute (180 rpm) for 3 days. The addition of 0.1% L-arabinose in the culture medium when the OD₆₀₀ is ≈0.4 allowed the expression of the gene present in the plasmid and the production of a ACL0729-(His)₆ fusion protein, of 96.9 kDa.

### 2. Production of β-1,2-Mannobiose, β-1,3-Mannobiose, β-1,4-Mannobiose and Laminaribiose (β-1,3-Glucobiose)

### 2.1. Production of β-1,2-Mannobiose

Overnight cultures (10 to 15 hours) in LB medium of the non-transformed E. coli PFKA1-pBAD-Teth-1788 and PFKA1 strains (control condition) were used to inoculate at a OD₆₀₀≈0.1 a culture of 50 mL of M9 medium supplemented with 3 g/L D-Mannose as the unique carbon source in a 250 mL baffled Erlenmeyer flask. The Erlenmeyer flasks were incubated for 24 h at 37° C. with an orbital stirring of 220 rpm. After 24 hours, the cells were harvested by centrifugation for 10 min at 2 000 g at room temperature, i.e. 24° C., washed by cell suspension in a M9 medium and used to inoculate at OD₆₀₀≈0.1 a 500 mL bioreactor containing 350 mL of “modified” M9 medium (M9 modified salts: KH₂PO₄ 3.02 g/L, NaCl 0.51 g/L, NH₄Cl 2.04 g/L, (NH₄)2SO₄ 5 g/L. Trace metals and salts: Na2EDTA 2 H₂O 15 mg/L, ZnSO₄ 7H₂O 4.5 mg/L, CoCl₂ 6H₂O 0.3 mg/L, MnCl2 4H₂O 1 mg/L, H₃BO₃ 1 mg/L, Na2MoO₄ 2 H₂O 0.4 mg/L, FeSO4 7 H₂O 3 mg/L, CuSO₄ 5 H₂O 0.3 mg/L. MgSO₄ 0.5 g/L CaCl₂) 4.38 mg/L, Thiamine hypochloride 0.1 g/L) supplemented with 10 g/L of D-mannose. The fermentation parameters, namely a pH of 7.0, a temperature of 37° C., a partial pressure of dissolved O₂ (pO2) of 30% or more, a stirring of 500 rpm or more were monitored and controlled by means of a Multifors bioreactor system (Infors, Switzerland). The growth was estimated by measuring the turbidimetry of the culture medium at an optical density of 600 nm using a Genesys 6 spectrophotometer (Thermo, USA). Culture samples of the PFKA1-pBAD-Teth-1788 strain and the PFKA1 control strain were collected at different culture times and analyzed by nuclear magnetic resonance (NMR).

### 2.2. Identification of Products by Nmr

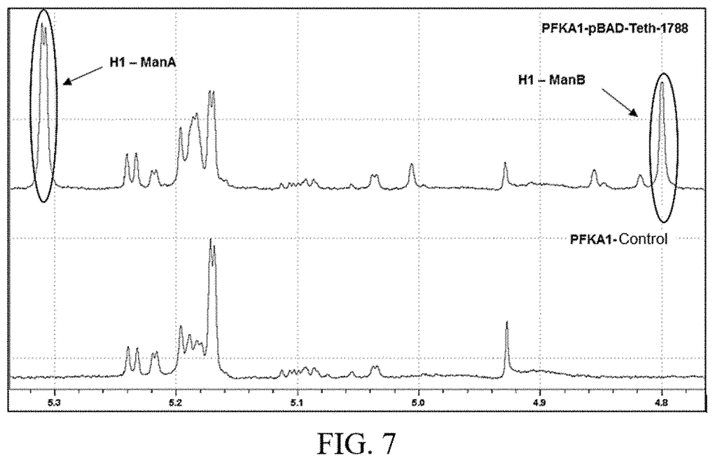

Regular samples of the culture medium (1 mL) were centrifuged for 2 min at 18 000 g and 500 μL of the supernatants were mixed with 100 μL of D₂O containing 2.35 g/L of tetra-deuterated 3-(trimethylsilyl)-1-propanesulfonic acid (TSPd4), compound used as an internal standard for NMR analysis. ¹H-NMR spectra were acquired with an Avance 500 MHz NMR spectroscope equipped with a 5 mm BBI probe (Bruker, Rheinstatten, Germany). The processing of the spectra and the quantification of the metabolites were carried out with the Topspin 3.1 software (Bruker, Rheinstatten, Germany). The culture supernatants obtained with the PFKA1-pBAD-Teth-1788 or PFKA1 strains were thus analyzed throughout the culture. FIG. 7 represents the NMR spectra of supernatants of culture medium obtained with the PFKA1-pBAD-Teth-1788 (upper curve) or non-transformed PFKA1 strains. As represented in FIG. 7, the characteristic peaks of β-1,2-mannobiose in the PFKA1-pBAD-Teth-1788 cultures were detected while none of these peaks was present in the PFKA1 control culture supernatant. In fact, the chemical shifts observed on the spectrum of the PFKA1-pBAD-Teth-1788 culture supernatants corresponded to the peaks of hydrogen atoms bonded to carbon 1 of the reducing part of β-1,2-mannobiose (H1 -ManA) and carbon 1 of mannose from the non-reducing end of β-1,2-mannobiose (H₁-ManB), given at 5.31 and 4.79 ppm respectively (Faille, C., Michalski, J. C., Strecker, G., Mackenzie, D. W., Camus, D., Poulain, D., 1990. Immunoreactivity of neoglycolipids constructed from oligomannosidic residues of the Candida albicans cell wall. Infect. Immun. 58, 3537-3544. [1], Shibata, N., Hisamichi, K., Kikuchi, T., Kobayashi, H., Okawa, Y., Suzuki, S., 1992. Sequential nuclear magnetic resonance assignment of. beta-1,2-linked mannooligosaccharides isolated from the phosphomannan of the pathogenic yeast Candida albicans NIH B-792 strain. Biochemistry 31, 5680-5686 [11]).

The obtained results clearly demonstrate that the strain according to the invention advantageously allows the production of oligosaccharides. In addition, the obtained results clearly demonstrate that the strain according to the invention advantageously and surprisingly and unexpectedly allows the excretion of oligosaccharides produced in the culture medium.

In other words, the results clearly demonstrate that the strain according to the invention allows the synthesis of oligosaccharides and advantageously their excretions in the culture medium.

2.3. Analysis of the Presence of β-1,2-Mannobiose in the Culture Supernatant by High-Performance Anion Exchange Chromatography with Pulsed Amperometry Detection (HPAEC-PAD)

Confirmation of the presence of β-1,2-mannobiose in the extracellular medium was carried out via an orthogonal analysis method. The analyzed samples corresponded to culture supernatants of the PFKA1 strain transformed with pBAD-Teth-1788 or of the PFKA1 strain as described above.

Samples of 1 mL of culture supernatants were subjected to heat shock for 10 min at 95° C., then centrifuged for 10 minutes at 15,000 g and were filtered on a 0.22 μm membrane. A 25-fold dilution of the filtered samples in ultrapure water was carried out before analysis. The separation of the molecules was carried out on a 2×250 mm PA100 Dionex Carbopac column (Thermo Fisher) with an elution gradient at 0.25 mL/min with the eluents A (H₂O), B (150 mM NaOH) and C (150 mM NaOH+500 mM sodium acetate) as follow: 0-3 min, 50% A-50% B; 3 min, 100% B; 3-6 min, 100% B; 6-12 min, 50% B-50% C; 12 min, 5% B-95% C; 12-15 min, 5% B-95% C; 15 min, 50% A-50% B; 15-23 min, 50% A-50% B. The detection was carried out by a Dionex ED40 module having a gold electrode and anAg/AgCl pH reference.

Furthermore, an in vitro production (without the strain) from an enzymatic reaction mixture with Teth-1788 β-1,2-mannobiose phosphorylase was carried out using 300 mM mannose, 15 mM α-D-Mannose 1-Phosphate, 0.16 mg·mL⁻¹ Teth-1788 enzyme in a total volume of 10 mL of 20 mM tris-HCl buffer, pH7. The reaction was carried out for 24 hours in a water bath at 37° C. under stirring. The obtained β-1,2-mannobiose was purified as described in Chiku K, Nihira T, Suzuki E, Nishimoto M, Kitaoka M, et al. (2014) Discovery of Two b-1,2Mannoside Phosphorylases Showing Different Chain-Length Specificities from Thermoanaerobacter sp. X-514. PLoS ONE 9(12): e114882. doi:10.1371/journal.pone.0114882 [61]

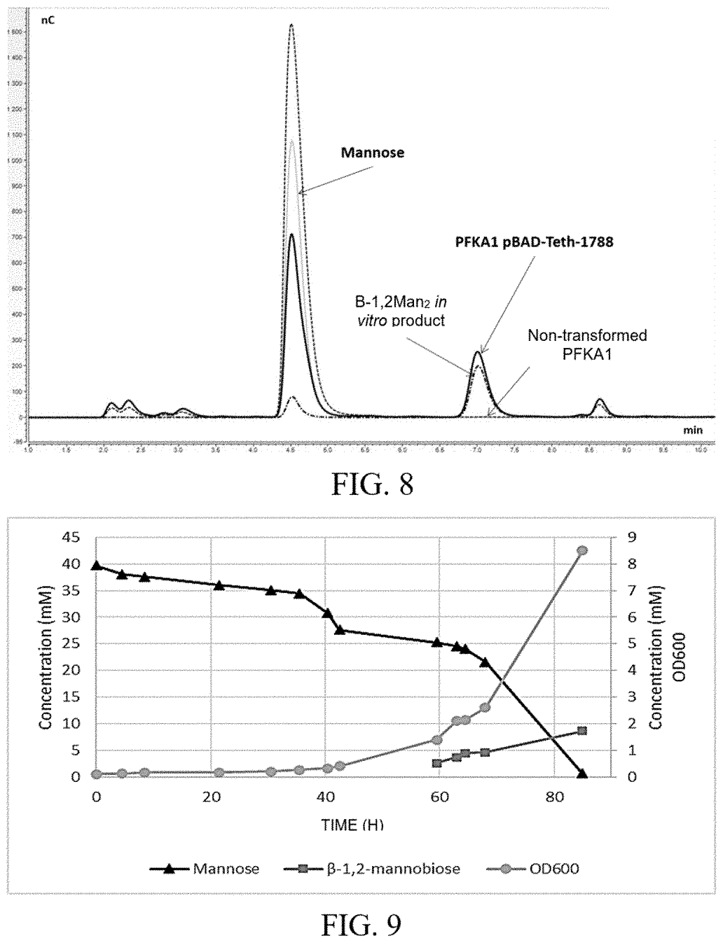

The obtained results, namely the chromatograms are represented in FIG. 8. In particular, in this figure, the bold solid line curve corresponds to the chromatogram of the culture supernatant of the PFKA1 strain transformed with pBAD-Teth-1788, the dotted curve corresponds to the chromatogram of the culture supernatant of the non-transformed PFKA1 strain, the thin solid line curve corresponds to a standard D-mannose (Carbosynth). As demonstrated in FIG. 8, the strain according to the invention advantageously allows the production of oligosaccharides. In addition, the obtained results demonstrate that the produced oligosaccharides are excreted in the medium advantageously allowing a facilitated recovery of the produced oligosaccharide.

### 2.4. Production Profile of β-1,2-Mannobiose in Bioreactor

A study of the production of β-1,2-mannobiose was followed by NMR during cultures in bioreactor with the MDO, MGX, MGX1 and PFKA1 strains.

The strains were transformed beforehand with the pBAD-Teth-1788 plasmid. The culture conditions of each of the strains were identical to those mentioned above. FIG. 9 shows the production profile of β-1,2-mannobiose by the PFKA1 strain transformed with the pBAD-Teth-1788 plasmid. On this FIG. 9 the evolution of the optical density at 600 nm and that of the concentration of mannose are also plotted.

The determination of the concentrations was carried out by NMR from supernatant samples of the culture medium according to the process described in Nord LI, Vaag P, Duus JØ Quantification of organic and amino acids in beer by 1H NMR spectroscopy Anal Chem. 2004 Aug. 15; 76(16):4790-8 [59] or Gloriadel Campo, InakiBerregi, RaúlCaracena, J. IgnacioSantos “Quantitative analysis of malic and citric acids in fruit juices using proton nuclear magnetic resonance spectroscopy” Analytica Chimica Acta, Volume 556, Issue 2, 25 Jan. 2006, Pages 462-468 [60] using 1 mM TSP (Trimethylsilylpropanoic acid) as an internal standard to calculate the concentration of β-1,2-mannobiose.

The final concentration of β-1,2-mannobiose in the culture medium was 1.80 mM and the yield of β-1,2-mannobiose per g of consumed mannose is 9.02% (g/g).

In the same way and by following the same protocol, the production of β-1,2-mannobiose was evaluated in bioreactor for the MDO, MGX and MGX1 strains, transformed with the pBAD-HisA-Teth514-1788 plasmid according to the process described above in order to determine the effect of each mutation on the production of β-1,2-mannobiose. Table 9 groups together the calculated production characteristics for the different strains from the concentrations of β-1,2-mannobiose and of residual mannose determined by NMR.

TABLE 9
TITERS AND YIELDS OF β-1,2-MANNOBIOSE IN THE
CULTURE SUPERNATANTS IN BIOREACTOR OF THE
MDO, MGX, MGX1 AND PFKA1 STRAINS
EXPRESSING THE TETH-1788 ENZYME.
THE CONCENTRATIONS ARE EXPRESSED
IN MM AND THE YIELDS IN A PERCENTAGE
OF MANNOSE CONVERTED INTO β-
1,2-MANNOBIOSE

| | | Man₂ Titer | % Yield (g |
|---|---|---|---|
| Strain | Characteristic | (mM) | Man₂/g Man) |
| MDO-Teth- | Control strain | ND | ND |
| 1788 | | | |
| MGX-Teth- | PTS-, no GalP; non-induced | 0.21 | 1.02 |
| 1788 | manB | | |
| MGX1- | PTS-with induced GalP, | 0.59 | 2.91 |
| Teth-1788 | induced manB | | |
| PFKA1- | PTS-with GalP; deletion of | 1.8 | 9.02 |
| Teth-1788 | pfka; induced manB | | |

ND. Not detected

As demonstrated above, in the culture medium of the MDO strain, the β-1,2-mannobiose is not detected while in the MGX strain, it is present in very low but quantifiable concentration (0.21 mM). Also, it seems that the deletion of the gene encoding the PTS allows the import of the mannose in the cell in a non-phosphorylated form, which is one of the glycoside-phosphorylase substrates. The MGX1 strain shows an increase in the β-1,2-mannobiose production (0.59 mM). This increase may be related to two factors: 1) improvement of the mannose transport by overexpression of GalP and 2) increase of the intracellular concentration of mannose-1-Phosphate (M1P) (in comparison with the MGX strain) due to overexpression of the manB gene, the product of which, the ManB protein, catalyzes the formation of M1P from Mannose-6-Phosphate (M6P). M1P is the second glycoside phosphorylase substrate and the intracellular level of M1P is important for the biosynthesis of β-1,2-mannobiose. Surprisingly and unexpectedly, the production of β-1,2-mannobiose by the PFKA1 strain is tripled with respect to the MGX1 strain (1.8 mM), which represents an increase by approximately a factor of 9 with respect to the MGX strain. Advantageously, the intracellular increase in M1P possibly has an effect in obtaining this result.

As demonstrated above, the strain according to the invention advantageously and surprisingly allows to significantly increase the production of oligosaccharides. In addition, the results obtained clearly demonstrate that the strain according to the invention allows to significantly increase the production yields of oligosaccharides and thus to optimize/reduce the production costs.

### Purification of in Cellulo Synthesized β-1,2-Mannobiose

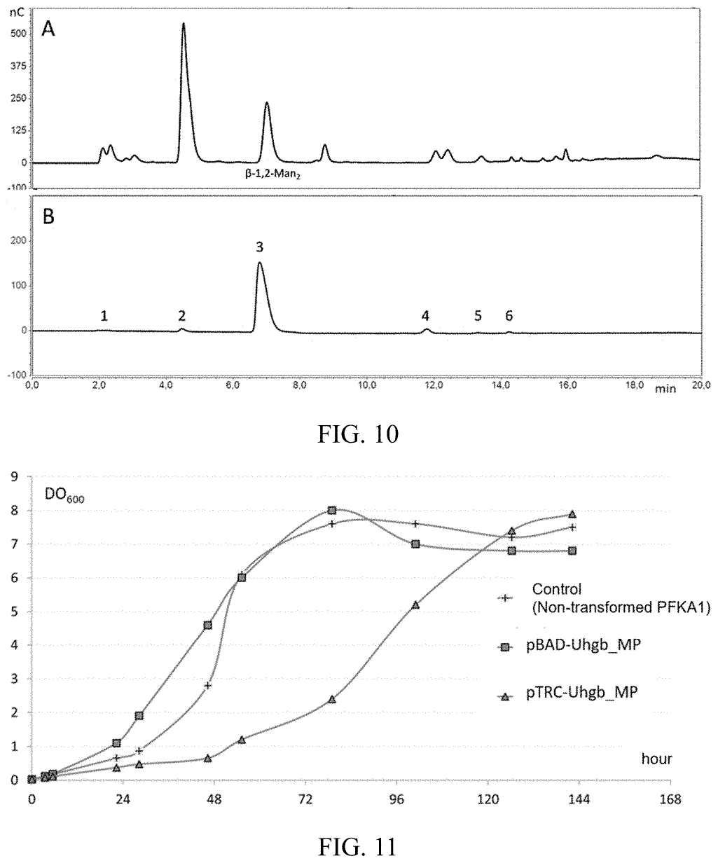

An analysis of the β-1,2-mannobiose produced by the PFKA1-pBAD-Teth1788 strain was carried out. To do this, 12 mL of culture supernatant of the PFKA1-pBAD-Teth1788 strain were subjected to a heat shock for 8 min at 95° C., centrifuged for 10 min at 15 000 g at 15° C. The supernatant was filtered through a membrane (sartorius, Minisart) in cellulose acetate with pores of 0.22 μm before being lyophilized and taken up in 500 μL of H₂O in total, in 2 vials with insert (250 μL each). The purification was carried out with a HPLC 1260 infinity (Agilent) coupled to an UltiMate 3000 automatic fraction collector (Thermo scientific). An asahipak NH2P-50 4E column (Shodex) allows the separation of the compounds via isocratic elution of an acetonitrile/H₂O mixture (70/30 respectively), at a flow rate of 1 mL/min. The compounds were detected by refractive index (RI). The purity of β-1,2-mannobiose was estimated before (FIG. 10A) and after purification (FIG. 10B). Two consecutive purification cycles allowed to obtain a HPLC purity ≥90% (Table 10). The purity was calculated by taking the ratio of the area under the peak of β-1,2-mannobiose and the area under all the peaks of the chromatogram.

TABLE 10
PERCENTAGE OF AREA UNDER THE CURVE

| peak | 1 | 2 | 3 | 4 | 5 | 6 |
|---|---|---|---|---|---|---|
| number | Contaminant | Contaminant | β-1,2-man₂ | Contaminant | Contaminant | Contaminant |
| Relative | 1.04 | 2.10 | 91.47 | 4.07 | 0.67 | 0.65 |
| area (%) | | | | | | |

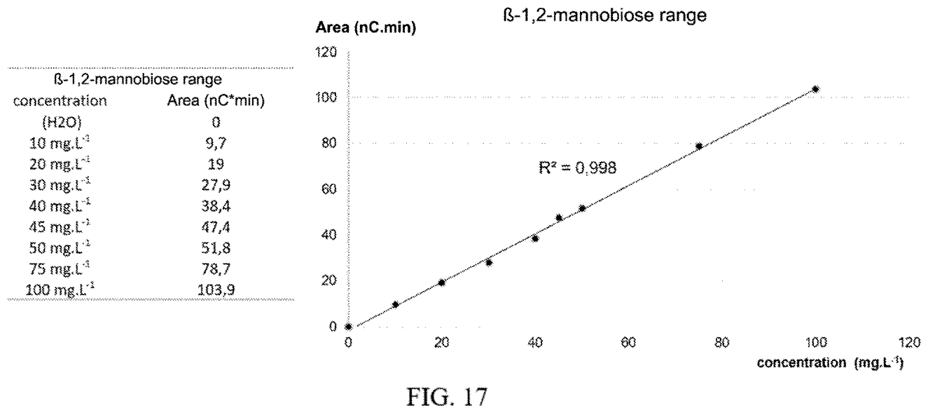

From the chromatograms, the obtained titers and purification yields were calculated from standard curves represented in FIG. 17. The process used corresponds to that described in Quantification of Sugar Compounds and Uronic Acids in Enzymatic Hydrolysates of Lignocellulose Using High-Performance Anion Exchange Chromatography with Pulsed Amperometric Detection Energy Fuels 2012, 26, 5, 2942-2947 [53]

Table 11 below summarizes the obtained yield and purification results.

TABLE 11
PURIFICATION YIELDS OF B-1,2-MANNOBIOSE FROM 12 ML
OF PFKA1-PBAD-TETH-1788 CULTURE SUPERNATANT.

| Culture supernatant volume | β-1,2-mannobiose (purified) mass |
|---|---|
| 12 mL | 6.8 mg |

Titer in the culture supernatant after purification: 567 mg · L⁻¹
Titer in the culture supernatant before purification, calculated from the NMR data,
950 mg · L⁻¹ and from the HPAEC-PAD data,
1260 mg · L⁻¹.
Purification yield: 45% (HPAEC-PAD), 60% (NMR)

As demonstrated above, the strain according to the invention advantageously allows to produce oligosaccharides in high yields. Moreover, as demonstrated above, the strain according to the invention advantageously allows the accumulation of the produced oligosaccharides in the culture medium. In addition, the recovery of the produced oligosaccharide is facilitated from the culture medium.

### 3. Production of β-1,4-Mannobiose

### 3.1. Production of β-1,4-Mannobiose and NMR Identification

Cultures of the non-transformed (control condition), transformed with the pBAD-UhgbMP plasmid or transformed with the pTRC-UhgbMP plasmid E. coli PFKA1 strain were produced in an Erlenmeyer flask according to the protocol described in Example 1. A study of the growth profile of these 3 cultures was carried out. Growth profile analysis was carried out as described above. FIG. 11 shows the growth profiles of the different cultures. As represented in this figure, the growth profiles are similar for the E. coli PFKA1 and PFKA1-pBAD-UhgbMP strains and show a plateau after 72 hours. This plateau is reached after 120 hours of culture as relating to the E. coli PFKA1-pTRC-UhgbMP strain.

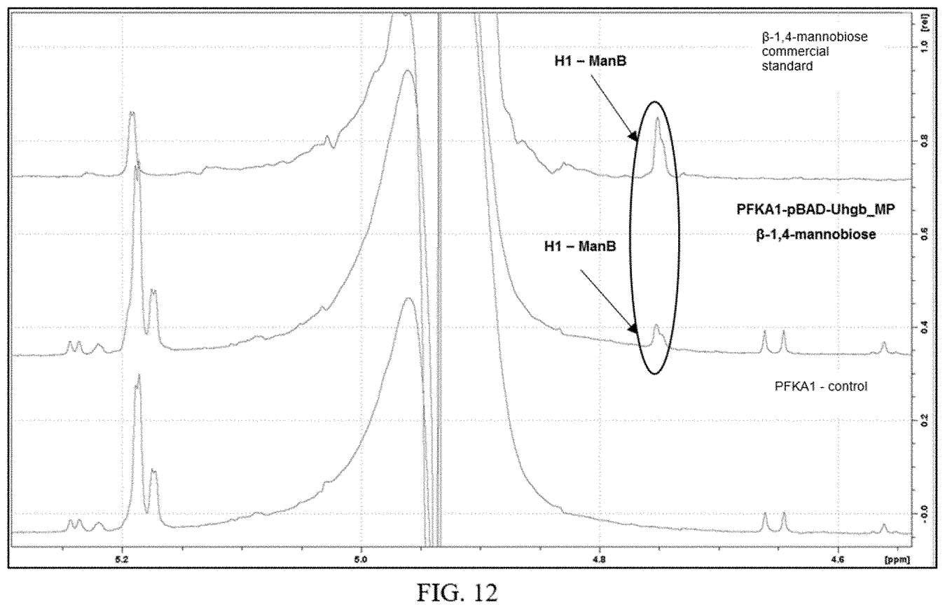

A culture supernatant with the E. coli PFKA1-pBAD-UhgbMP strain was analyzed by NMR by applying the same parameters as those previously described. FIG. 12 shows the characteristic spectral zone of the β-1,4-mannobiose of a supernatant collected from a culture of the E. coli PFKA1 strain without the plasmid (control condition), a culture supernatant of the E. coli PFKA1_pBAD-UhgbMP strain and a solution of the commercial standard β-1,4-mannobiose (Carbosynth, UK). As represented in FIG. 12, the chemical shift at 4.76 ppm is characteristic of the β-1,4-mannobiose. This is the hydrogen atom bonded to carbon 1 of the non-reducing part of β-1,4-mannobiose (H₁-ManB). This peak is also present in the spectrum of the culture supernatant of the E. coli PFKA1-pBAD-UhgbMP strain whereas it is absent in that of the E. coli PFKA1 strain. The obtained results therefore clearly demonstrate that the transformed strain advantageously allows to produce oligosaccharides, in particular the β-1,4-mannobiose. In addition, this example clearly demonstrates that the strain allows the production of oligosaccharides which are excreted in the culture medium.

The obtained results therefore clearly demonstrate that the strain according to the invention advantageously allows the production of oligosaccharides. In addition, the obtained results clearly demonstrate that the strain according to the invention advantageously and surprisingly and unexpectedly allows the excretion of oligosaccharides produced in the culture medium.

In addition, the results clearly demonstrate that the strain according to the invention allows the synthesis of oligosaccharides and advantageously their excretion in the culture medium. Thus the production, recovery and isolation of oligosaccharides do not require any alteration or destruction of the strain, advantageously allowing a continuous production.

### 3.2. Confirmation of the Production β-1,4-Mannobiose by HPAEC-PAD

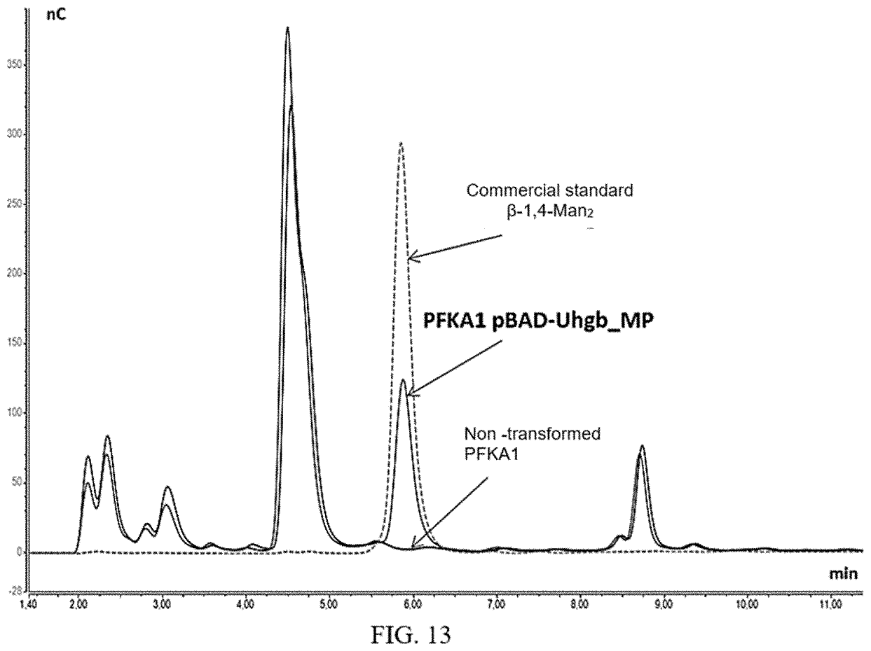

The samples were analyzed by HPAEC-PAD by applying the same protocol as that described previously (see section 3). FIG. 13 presents the chromatograms of the different samples analyzed by this technique. In particular, in this figure, the thin solid line curve corresponds to the chromatogram of the culture supernatant of the PFKA1 strain transformed with pBAD-Teth-1788, the bold solid line curve corresponds to the chromatogram of the culture supernatant of the non-transformed PFKA1 strain, the dotted curve corresponds to a commercial standard β-1,4-mannobiose (Carbosynth).

As demonstrated in FIG. 13, the strain according to the invention advantageously allows the production of oligosaccharides. In addition, the obtained results demonstrate that the produced oligosaccharides are excreted in the medium advantageously allowing a facilitated recovery of the produced oligosaccharide.

As represented in FIG. 13, the β-1,4-mannobiose is present only in the culture supernatant of the PFKA1 strain transformed with pBAD-UhgbMP and therefore confirms the production of the β-1,4-mannobios by the PFKA1-pBAD-UhgbMP strain.

In addition, the obtained results demonstrate that the produced oligosaccharides are excreted in the medium advantageously allowing a conservation of the strain/culture and also advantageously an easier isolation/recovery of the produced oligosaccharide.

### 4. Production of B-1,3-Mannobiose

### 4.1. Production of β-1,3-Mannobiose and NMR Identification

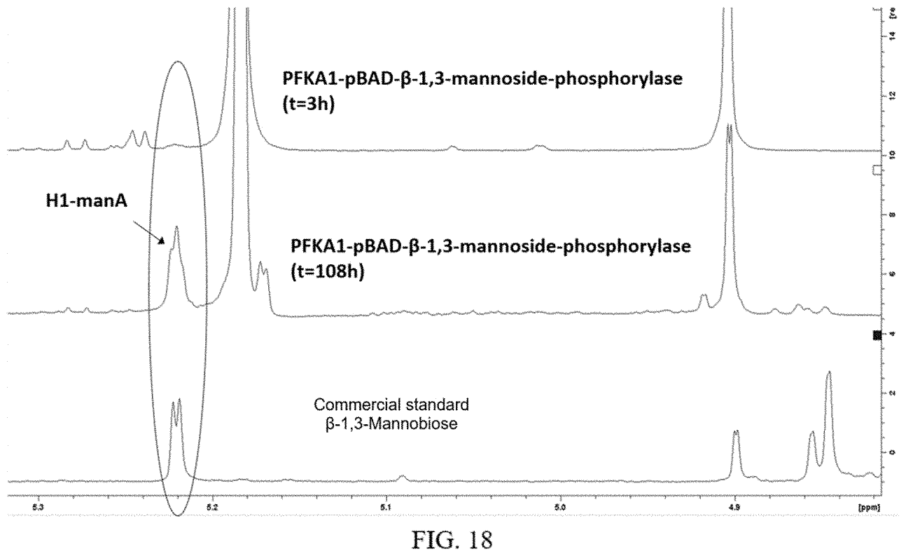

A culture of the E. coli PFKA1 strain transformed with the pBAD-β-1,3-mannooligosaccharide phosphorylase plasmid was carried out in an Erlenmeyer flask according to the protocol described in Example 1. The culture supernatant with the PFKA1-pBAD-β-1,3-mannooligosaccharide phosphorylase strain was analyzed by NMR at the beginning (3 hours) and at the end of culture (4 days) by applying the same parameters as those previously described. FIG. 18 shows the characteristic spectral zone of the δ-1,3-mannobiose of a culture supernatant of the E. coli PFKA1-pBAD-β-1,3-mannooligosaccharide phosphorylase strain after 3 or 108 hours of culture and a solution of the commercial standard δ-1,3-mannobiose (Carbosynth, UK). As represented in FIG. 18, the chemical shift at 5.22 ppm is characteristic of the β-1,3-mannobiose. This is the hydrogen atom bonded to carbon 1 of the reducing part of β-1,3-mannobiose (H1-ManA). This peak is also present in the spectrum of the culture supernatant of the E. coli PFKA1_pBAD-β-1,3-mannooligosaccharide phosphorylase at the end of the culture whereas it is absent in that of the same strain at the beginning of the culture. The obtained results therefore clearly demonstrate that the transformed strain advantageously allows to produce oligosaccharides, in particular the β-1,3-mannobiose. In addition, this example clearly demonstrates that the strain allows the production of oligosaccharides which are excreted in the culture medium.

The obtained results therefore clearly demonstrate that the strain according to the invention advantageously allows the production of oligosaccharides. In addition, the obtained results clearly demonstrate that the strain according to the invention advantageously and surprisingly and unexpectedly allows the excretion of oligosaccharides produced in the culture medium.

In addition, the results clearly demonstrate that the strain according to the invention allows the synthesis of oligosaccharides and advantageously their excretion in the culture medium.

### 4.2. Confirmation of the Production β-1,3-Mannobiose by HPAEC-PAD

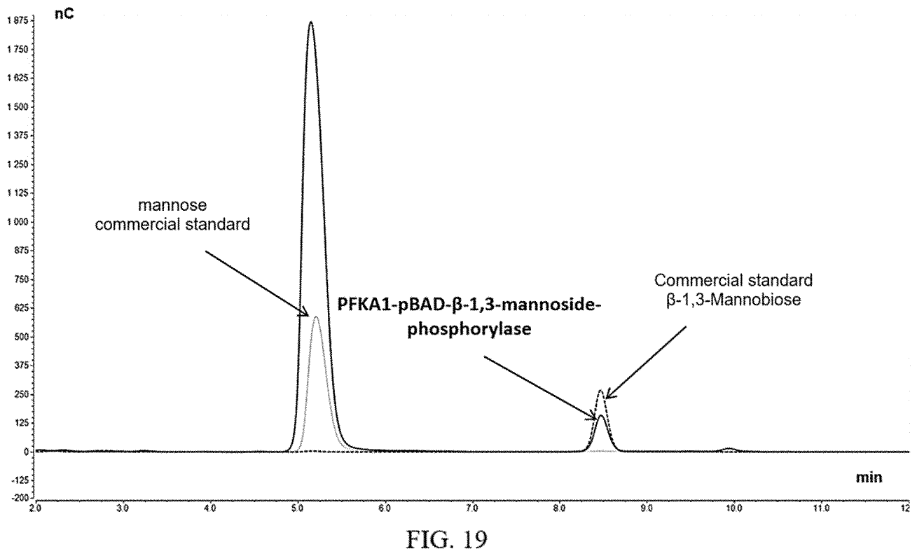

The samples were analyzed by HPAEC-PAD by applying the same protocol as that described previously (see section 3). FIG. 19 presents the chromatograms of the different samples analyzed by this technique. In particular, in this figure, the bold solid line curve corresponds to the chromatogram of the culture supernatant of the PFKA1 strain transformed with pBAD-β-1,3-mannooligosaccharide phosphorylase after 108 hours of culture, the dotted curve corresponds to the chromatogram of a standard de δ-1,3-mannobiose (Carbosynth), the thin solid line curve corresponds to a standard d-mannose (Carbosynth).

As demonstrated in FIG. 19, the strain according to the invention advantageously allows the production of oligosaccharides. In addition, the obtained results demonstrate that the produced oligosaccharides are excreted in the medium allowing a facilitated recovery of the produced oligosaccharide.

As represented in FIG. 19, the δ-1,3-mannobiose is present in the culture supernatant of the PFKA1 strain transformed with pBAD-β-1,3-mannooligosaccharide phosphorylase and therefore confirms the production of the β-1,3-mannobiose by the PFKA1-pBAD-β-1,3-mannooligosaccharide phosphorylase strain.

As demonstrated in FIG. 19, the strain according to the invention advantageously allows the production of oligosaccharides. In addition, the process advantageously allows synthesis and/or production of oligosaccharides at a much lower cost with respect to in vitro enzymatic processes, not using a living biological support. In addition, the obtained results demonstrate that the produced oligosaccharides are excreted in the medium advantageously allowing a facilitated recovery of the produced oligosaccharide.

### 5. Production of Laminaribiose (β-1,3-Glucobiose)

### 5.1. Production of Laminaribiose and NMR Identification

The E. coli PFKA1 strain transformed with the pBAD-His-laminaribiose phosphorylase plasmid (pBAD-ACL0729) according to the classic protocol for transforming chemo-competent cells (https://www.addgene.org/protocols/bacterial-transformation/[50]) and the non-transformed PFKA1 strain were cultured in an Erlenmeyer flask according to the protocol described in Example 2 paragraph 1.4.

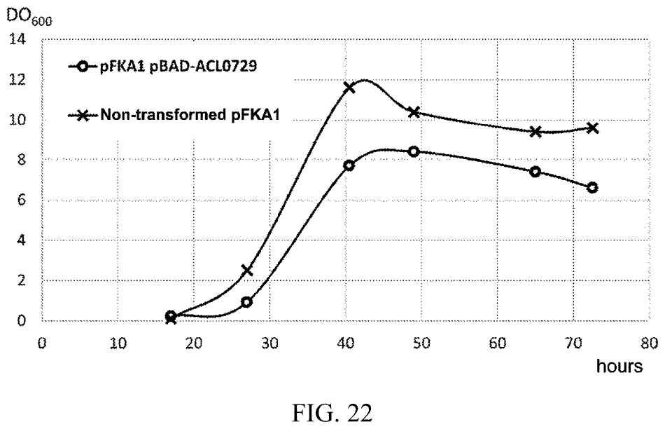

FIG. 22 represents the growth profiles of two cultures. As represented in this figure, the growth profiles are similar for the E. coli PFKA1 and PFKA1-pBAD-ACL0729 strains and reach a plateau after 40 hours.

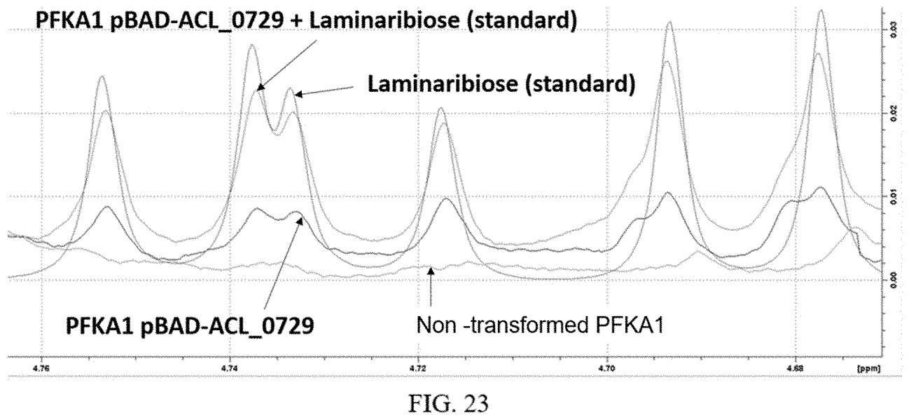

The supernatants of the two cultures, with the PFKA1-pBAD-ACL0729 strain and non-transformed pFKA1 were analyzed by NMR after 49 hours of culture and compared by applying the same parameters as those previously described. FIG. 23 shows a spectral region between 4.76 and 4.67 ppm where signals are present only in the culture supernatant of the E. coli PFKA1-pBAD-ACL0729 strain and commercial laminaribiose (Carbosynth, UK) but not in the culture supernatant with non-transformed PFKA1. An addition of commercial laminaribiose in the culture supernatant of the E. coli PFKA1-pBAD-ACL0729 strain allows to observe an increase in these same signals, thus confirming the presence of laminaribiose in the supernatant of the culture of the E. coli PFKA1-pBAD-ACL0729 strain. The obtained results therefore clearly demonstrate that the transformed strain advantageously allows to produce oligosaccharides, in particular the laminaribiose. In addition, this example clearly demonstrates that the strain allows the production of oligosaccharides which are excreted in the culture medium.

The obtained results therefore clearly demonstrate that the strain according to the invention advantageously allows the production of oligosaccharides. In addition, the obtained results clearly demonstrate that the strain according to the invention advantageously and surprisingly and unexpectedly allows the excretion of oligosaccharides produced in the culture medium.

In addition, the results clearly demonstrate that the strain according to the invention allows the synthesis of oligosaccharides and advantageously their excretion in the culture medium.

### 5.2. Confirmation of the Production Laminaribiose by HPAEC-PAD

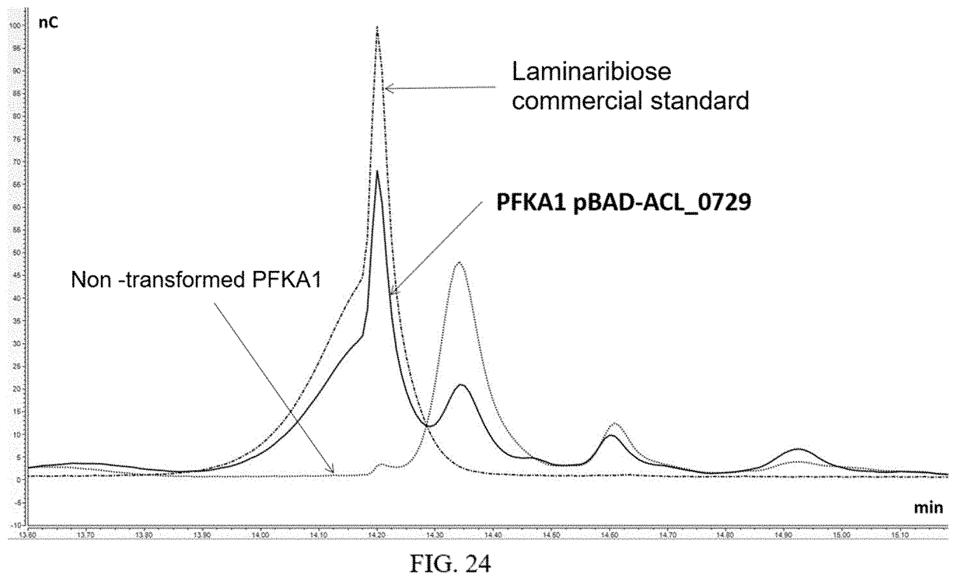

The samples were analyzed by HPAEC-PAD by applying the same protocol as that described above (Example 2 paragraph 3). FIG. 24 represents the chromatograms of the different samples analyzed by this technique. In particular, in this figure, the solid line curve corresponds to the chromatogram of the supernatant of the culture of the E. coli PFKA1 strain transformed with pBAD-ACL0729 after 49 hours of culture, the dotted curve corresponds to the chromatogram of the supernatant of the culture of the non-transformed E. coli PFKA1 strain, and the discontinuous dotted curve corresponds to a commercial standard laminaribiose (Carbosynth, UK),

As demonstrated in FIG. 24, the strain according to the invention advantageously allows the production of oligosaccharides. In addition, the obtained results demonstrate that the produced oligosaccharides are excreted in the medium allowing a facilitated recovery of the produced oligosaccharide.

As represented in FIG. 24, the laminaribiose is present only in the culture supernatant of the E. coli PFKA1 strain transformed with pBAD-ACL-0729 and therefore confirms the production of the laminaribiose by the E. coli PFKA1-pBAD-ACL-0729 strain.

Table 12 corresponds to the integration of the peaks obtained by HPAEC-PAD on a standard range of commercial laminaribiose (std Laminaribiose) at different concentrations: 1 mg/L, 5 mg/L, 10 mg/L, 50 mg/L or 100 mg/L (laminaribiose commercial standard calibrator range from 1 to 100 mg), on samples of supernatants of cultures of the non-transformed E. coli PFKA1 strain (PFKA1) at different culture times: 40 hours, 49 hours or 65 hours and on samples of supernatant of the cultures of the E. coli PFKA1 strain transformed with the pBAD-ACL0729 plasmid (PFKA1 pBAD-ACL0729) at different culture times: 40 hours, 49 hours or 65 hours. Culture supernatant samples were diluted 20 times before measurement.

TABLE 12
INTEGRATION OF THE PEAKS OBTAINED BY HPAEC-PAD AND THE
CORRESPONDING AMOUNT OF LAMINAROBIOSE.

| | Ret.Time | Area | Quantity | Quantity |
|---|---|---|---|---|
| | min | nC*min | (mg · L⁻¹) | (mM) |
| | ED_1 | ED_1 | ED_1 | ED_1 |
| Injection name | Laminaribiose | Laminaribiose | Laminaribiose | Laminaribiose |
| PFKA1 pBAD- | 14.19 | 7.01 | 6.63 | 0.019 |
| ACL0729 40 h | | | | |
| (dil20) | | | | |
| PFKA1 pBAD- | 14.19 | 7.28 | 6.93 | 0.020 |
| ACL0729 49 h | | | | |
| (dil20) | | | | |
| PFKA1 pBAD- | 14.20 | 6.92 | 6.52 | 0.019 |
| ACL0729 65 h | | | | |
| (dil20) | | | | |
| std | 14.19 | 88.09 | 98.66 | 0.288 |
| Laminaribiose | | | | |
| 100 mg/l | | | | |
| std | 14.20 | 47.61 | 52.70 | 0.154 |
| Laminaribiose 50 | | | | |
| mg/l | | | | |
| std | 14.20 | 10.27 | 10.32 | 0.030 |
| Laminaribiose 10 | | | | |
| mg/l | | | | |
| std | 14.18 | 5.08 | 4.44 | 0.013 |
| Laminaribiose 5 | | | | |
| mg/l | | | | |
| std | 14.18 | 1.06 | n.d. | n.d. |
| Laminaribiose 1 | | | | |
| mg/l | | | | |
| PFKA1_40 h | n.d. | n.d. | n.d. | n.d. |
| (dil20) | | | | |
| PFKA1_49 h | 14.19 | 0.10 | n.d. | n.d. |
| (dil20) | | | | |
| PFKA1_65 h | 14.21 | 0.04 | n.d. | n.d. |
| (dil20) | | | | |

In Table 12 above “Ret. Time” means retention time and “n.d.” not determined.

As demonstrated in Table 12, after 49 hours of culture, the culture supernatant of the E. coli PFKA1-pBAD-ACL-0729 strain shows a laminaribiose titer of 139 mg·L⁻¹. As demonstrated in FIG. 24 and in Table 12, the strain according to the invention advantageously allows the production of oligosaccharides. In addition, the process advantageously allows synthesis and/or production of oligosaccharides at a much lower cost with respect to in vitro enzymatic processes, not using a living biological support. In addition, the obtained results demonstrate that the produced oligosaccharides are excreted in the medium advantageously allowing a facilitated recovery of the produced oligosaccharide.

### Example 3: Manufacture and Characterization of the Strain Filed with the CNCM Under Number CNCM I-5681

From the strain described in Example 1, the following modifications were provided to obtain further improved yields for the conversion of mannose into mannobiose. In the example below, the strain filed with the CNCM (Collection Nationale de Culture de Microorganismes, Institut Pasteur, 25 rue du Docteur Roux, 75724 Paris Cedex 15, France), under number CNCM I-5681 is also designated CS1 or CS0ΔmaaΔmanA or OLI-CS1 strain.

1. Preparation and Production of the Sc1 Strain Filed with the CNCM Under Number CNCM I-5681 and Phenotypic Validation

### 1.1 Deletion of the Maa Gene:

The production of mannobiose with the E. coli PFKA1 strain, also referred to as CS0, cultured in minimal medium, comes along with the production of a compound, detected by NMR. This compound would correspond to an acetylated sugar, formed in the cell cytosol by maltose acetyltransferase, encoded by the maa gene (Leila Lo Leggio, Florence Dal Degan, Peter Poulsen, Søren Møller Andersen, and Sine Larsen. The Structure and Specificity of Escherichia coli Maltose Acetyltransferase Give New Insight into the LacA Family of Acyltransferases. Biochemistry 2003, 42, 18, 5225-5235; DOI: 10.1021/bi0271446 [63]). This enzyme is known to be able to add an acetyl group to a wide range of sugars. This reaction is made possible by the particular physiology of the PFKA1 (CS0) strain since the inactivation of genes encoding PTS system elements leads to the internalization of monosaccharides without chemical modification. In order to eliminate this contaminant, the maa gene was inactivated according to the protocol described in Datsenko and Wanner (Kirill A. Datsenko, Barry L. Wanner. One-step inactivation of chromosomal genes in Escherichia coli K-12 using PCR products. Proceedings of the National Academy of Sciences June 2000, 97 (12) 6640-6645; DOI: 10.1073/pnas.120163297 [62]). A DNA fragment containing the kanamycin resistance gene and flanked by Flippase Recognition Target (FRT) sites was amplified by adding to the ends of the two primers, 50 bp corresponding to the 5′ and 3′ ends of the maa gene. The primers used for PCR amplification were as follows:

FW:
(SEQ ID NO: 39)
5′ ATGAGCACAGAAAAAGAAAAGATGATTGCTGGTGAGTTGTATCGCTC
GGCGTGTAGGCTGGAGCTGCTTC 3′
and
RV:
(SEQ ID NO 40)
5′ TTACAATTTTTTAATTATTCTGGCTGGATTACCGCCCACGACAACGT
TGTCATATGAATATCCTCCTTAG 3′

The NEB phusion DNA polymerase was used to amplify the cassette using 1 ng of plasmid as a template, according to the manufacturer's instructions. The PCR amplification was carried out as follows: 1 cycle of 1 min at 98° C., 5 cycles of 30 sec at 98° C., 30 sec at 55° C. and 1 min at 72° C., 30 cycles of 30 sec at 98° C., 1 min at 72° C. and 1 cycle of 5 min at 72° C. The fragment thus amplified by PCR was purified from a gel and then quantified with a Nanodrop spectrophotometer.

The E. Coli PFKA1 (CS0) strain was chemically transformed with the pKD46 plasmid at 30° C. on LB agars supplemented with ampicillin at 50 μg/ml. The pKD46 plasmid possesses lambda, R and exo phage genes under the control of the arabinose promoter. Transformants carrying the pKD46 plasmid were cultured in 5 mL of LB medium containing 50 μg/mL of ampicillin and L-arabinose (0.2% w/v) at 30° C. to an OD₆₀₀ of 0.6, then concentrated 100 times and washed three times with 10% cold glycerol. Electroporation of cells as obtained, was carried out using a Cell-Porator with a voltage amplifier and 0.1 cm chambers according to the manufacturer's instructions, using 50 μL of cells and 200 ng of the PCR fragments obtained above. One milliliter of SOC medium (2% tryptone; 0.5% yeast extract; 10 mM NaCl, 2.5 mM KCl, 10 mM MgCl₂, 10 mM MgSO₄, and 20 mM glucose) was added to the cells having undergone this treatment and, incubated for 2 hours at 37° C., then spread on a LB agar supplemented with 50 μg/mL of kanamycin to select the Km transformants. The elimination of the maa gene was confirmed by PCR on colony as described below using the following primers:

| | FW: |
|---|---|
| (SEQ ID NO: 41) | |
| | 5′ GATGATTGCTGGTGAGTTGTATCG 3′ |
| | and |
| | RV: |
| (SEQ ID NO: 42) | |
| | 5′ TTAATTATTCTGGCTGGATTACCG 3′ |

The NEB taq DNA polymerase was used to amplify a fragment of the maa gene using 1 μL of a cell colony dissolved in 50 μL of sterile water according to the manufacturer's instructions. The PCR amplification was carried out as follows: 1 cycle of 5 min at 95° C., 35 cycles of 30 sec at 95° C., 30 sec at 55° C. and 1.5 min at 68° C., and 1 cycle of 5 min at 68° C. Once the colonies having lost the gene were identified, the elimination of the kanamycin resistance cassette was carried out by transforming, one of the positive colonies with the pCP20 plasmid. The plasmid pCP20 plasmid is an ampicillin-resistance plasmid with temperature sensitive replication and an induction of the FLP synthesis by heat shock. The Kanamycin-resistant (KmR) mutants were transformed with the pCP20 plasmid, and the ampicillin-resistant transformants were selected at 30° C. Transformants were isolated on LB agar non-selectively at 43° C. and then tested for loss of all antibiotic resistances.

The PFKA1 strain, also designated CS0 in which the maa gene has been inactivated, is referred to as CS0Δmaa.

### 1.2 Strain Phenotypic Validation

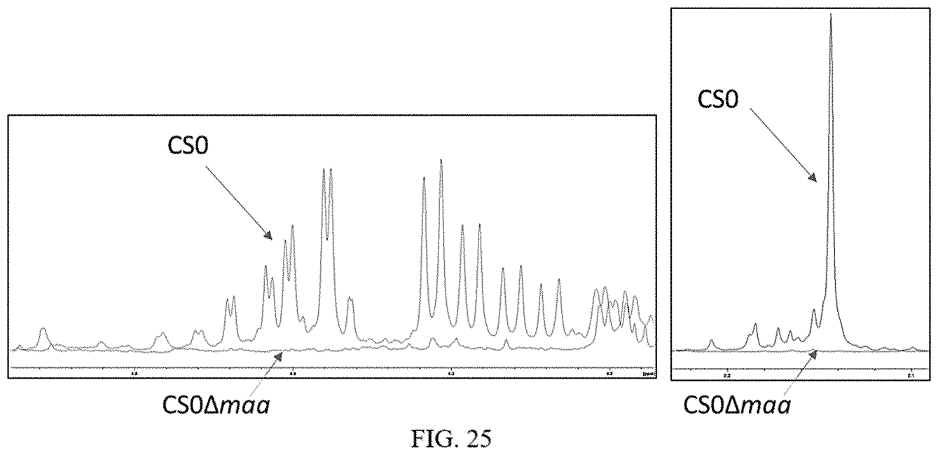

The E. coli PFKA1, also referred to as PFKA1, E. coli CS0 or CSO, and CS0Δmaa carrying the pBAD-Teth1788 plasmid were cultured in M9 mineral medium supplemented with mannose at a concentration of 6 g/L. The cultures were carried out in duplicate or in triplicate in 250 mL baffled Erlenmeyer flasks containing 50 ml of culture medium, at 37° C. and under orbital stirring at 200 rpm. The growth was followed by measuring turbidimetry at 600 nm. Kanamycin and ampicillin were added to a final concentration of 50 μg/mL, to ensure maintenance of the plasmids in the cells. IPTG (final concentration of 60 μM) and arabinose (final concentration of 10 mM) were added to the medium to induce the protein expression. Extracellular metabolites were identified and quantified by NMR. Samples of the culture were collected at different culture times and centrifuged for 2 min at 18,000 g. 500 μL of those supernatants were mixed with 100 μL of D₂O containing 2.35 g/L of tetra-deuterated 3-(trimethylsilyl)-1-propanesulfonic acid (TSPd4), molecule used as an internal standard for NMR analysis. ¹H-NMR spectra were acquired with an Avance 500 MHz NMR spectroscope equipped with a 5 mm BBI probe (Bruker, Rheinstatten, Germany). The processing of the spectra and the quantification of the metabolites were carried out with the Topspin 3.1 software (Bruker, Rheinstatten, Germany). The obtained results are represented in FIG. 25

As represented in FIG. 25, the NMR spectra obtained from samples of taken culture medium clearly demonstrate the presence of the contaminating product, namely an acetylated sugar, only in the sample stem from the culture medium of the CS0 (PFKA1) strain. In addition, the NMR spectrum obtained for the CS0Δmaa strain does not show this contaminant, acetylated sugar, and validates the genetic construction of the CS0Δmaa strain.

This example clearly demonstrates that the CS0Δmaa strain advantageously allows the production of oligosaccharides, for example of mannobiose, without the production of acetylated sugar.

### 1.3. Deletion of the Mana Gene

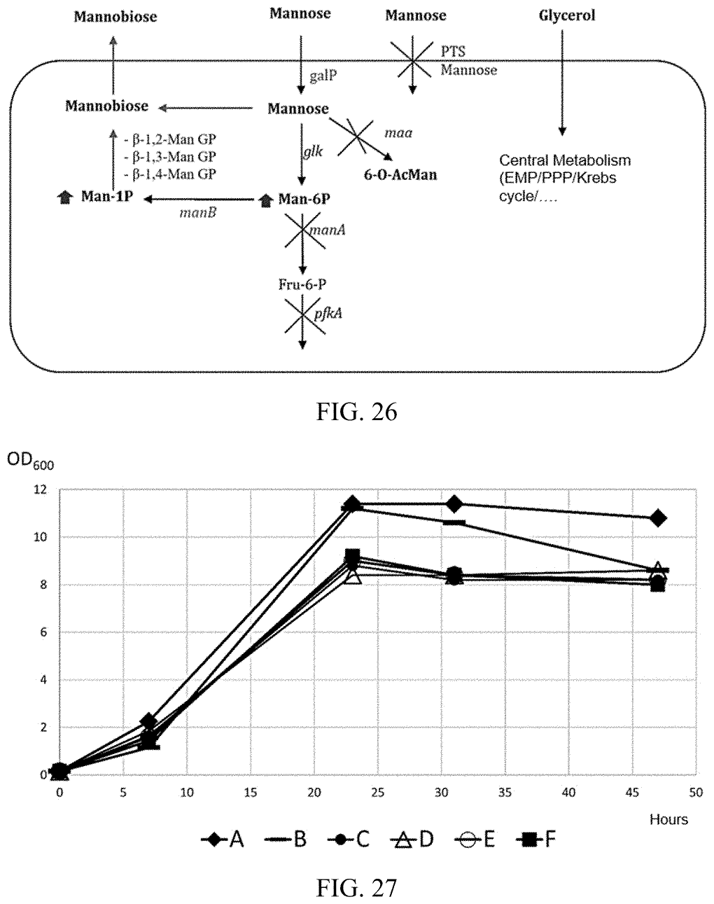

The inventors have surprisingly demonstrated that the deletion of the manA gene advantageously allows to maximize the conversion of mannose into mannobiose. FIG. 26 shows the general mannobiose production strategy in this example. This is a strain metabolic scheme for the production of mannobiose from glycerol and mannose. When mannose is the unique carbon source, it is also the energy source and the substrate for the synthesis of mannobiose. Therefore, the maximum conversion yield of mannose into mannobiose is severely limited. To overcome this limitation, the use of mannose for growth purposes can be greatly reduced by removing the manA gene, which catalyzes the conversion of mannose-6-phosphate into fructose-6-phosphate, a central metabolic intermediate, and adding another carbon and energy source, less expensive than mannose and not interacting with the production of mannobiose. Glycerol was therefore chosen and added as a carbon and energy source.

The deletion of the manA gene was carried out according to the Datsenko and Wanner protocol (Kirill A. Datsenko, Barry L. Wanner. One-step inactivation of chromosomal genes in Escherichia coli K-12 using PCR products. Proceedings of the National Academy of Sciences June 2000, 97 (12) 6640-6645; DOI: 10.1073/pnas.120163297 [62]). A DNA fragment containing the kanamycin resistance gene and flanked by “Flippase Recognition Target” (FRT) sites was amplified by adding to the ends of the two primers, 50 bp corresponding to the 5′ and 3′ ends of the manA gene. The primers used for PCR amplification were as follows:

FW:
(SEQ ID NO: 43)
5′ ATTATGCGCAGCACAGCCACTCTCCATTCAGGTTCATCCAAACAAAC
ACAGTGTAGGCTGGAGCTGCTTC 3′
and
RV:
(SEQ ID NO: 44)
5′ ACACGCGCTAAACGGCCGTGGCCTTTGACAGTCACCGGTGATTCGTT
GGCCATATGAATATCCTCCTTAG 3′

The NEB phusion DNA polymerase was used to amplify the cassette using 1 ng of pKD4 plasmid as a template, according to the manufacturer's instructions. The PCR amplification was carried out as follows: 1 cycle of 1 min at 98° C., 5 cycles of 30 sec at 98° C., 30 sec at 55° C. and 1 min at 72° C., 30 cycles of 30 sec at 98° C., 1 min at 72° C. and 1 cycle of 5 min at 72° C. The fragment thus amplified by PCR was purified from a gel and then quantified with a Nanodrop spectrophotometer.

The CS0Δmaa strain was transformed with the pKD46 plasmid at 30° C. on LB agars supplemented with ampicillin at 50 μg/ml. Transformants carrying the pKD46 plasmid were cultured in 5 mL of LB medium containing 50 μg/mL of ampicillin and L-arabinose (0.2% w/v) at 30° C. to an OD₆₀₀ of 0.6, then concentrated 100 times and washed three times with 10% cold glycerol. Electroporation of cells as obtained, was carried out using a Cell-Porator with a voltage amplifier and 0.1 cm chambers according to the manufacturer's instructions, using 50 μL of cells and 200 ng of the PCR fragments obtained above. 1 ml of SOC medium (see composition above) was added to the cells having undergone this treatment and, incubated for 2 hours at 37° C., then spread on a LB agar supplemented with 50 μg/rnL of kanamycin to select the Km transformants. The elimination of the manA gene was confirmed by PCR on colony (according to the process below) using the following primers:

| | FW: |
|---|---|
| (SEQ ID NO: 45) | |
| | 5′ GTGCGGGTCTGACGCCTAAATAC 3′ |
| | and |
| | RV: |
| (SEQ ID NO: 46) | |
| | 5′ GATGGGTTTCAATCTTCTTGCCT 3′. |

The NEB taq DNA polymerase was used to amplify a fragment of the manA gene using 1 μL of a cell colony dissolved in 50 μL of sterile water according to the manufacturer's instructions. The PCR amplification was carried out as follows: 1 cycle of 5 min at 95° C., 35 cycles of 30 sec at 95° C., 30 sec at 55° C. and 1.5 min at 68° C., and 1 cycle of 5 min at 68° C. Once the colonies having lost the gene were identified, the elimination of the kanamycin resistance cassette was carried out by transforming, one of the positive colonies with the pCP20 plasmid. The Kanamycin-resistant (KmR) mutants were transformed with the pCP20 plasmid, and the ampicillin-resistant transformants were selected at 30° C. Transformants were isolated on LB agar non-selectively at 43° C. and then tested for loss of all antibiotic resistances.

The strain thus obtained corresponds to the PFKA1 strain, also referred to as CSO, in which the maa gene and the manA gene have also been inactivated.

The PFKA1 strain, also referred to as CSO, in which the maa gene and the manA gene have been inactivated is referred to as CS0ΔmaaΔmanA or CS1.

1.4. Construction of the pTC-galP Plasmid

Induction of galP by IPTG in a high copy plasmid (pWKS) results in a significant additional energy cost for the bacteria, which can affect the cell function. Therefore, the galP gene was introduced into a medium copy plasmid under the control of a strong constitutive promoter. For this purpose, the pTC-galP plasmid was constructed, using the constitutive promoter of E. coli pIHF and the pA15 replication origin. The plasmid cloning was carried out by homologous recombination by amplifying 3 different DNA fragments with homologous ends to assemble the different elements of the plasmid.

The ADN fragments were amplified as follows:

a) Replication origin and kanR using the pZA23 plasmid as a template with the following primers:

| | FW: |
|---|---|
| (SEQ ID NO: 47) | |
| | 5′ AAATAGGCGCTCACGATTAAAAGGAAGCTGAGTTGGCTGC 3′ |
| | RV: |
| (SEQ ID NO 48) | |
| | 5′ TAGCTTTGCACTGTTTCAGAGTGAAGACGAAAGGGCCTCG 3′ |

The NEB phusion DNA polymerase was used to amplify the cassette using 1 ng of pKD4 plasmid as a template, according to the manufacturer's instructions. The PCR amplification was carried out as follows: 1 cycle of 1 min at 98° C., 5 cycles of 30 sec at 98° C., 30 sec at 60° C. and 1 min at 72° C., 30 cycles of 30 sec at 98° C., 1 min at 72° C. and 1 cycle of 5 min at 72° C. The fragment thus amplified by PCR was purified from a gel and then quantified with a Nanodrop spectrophotometer.

b) IHF promoter of the pBS1C3-IHF plasmid with the following primers:

| | FW: |
|---|---|
| (SEQ ID NO: 49) | |
| | 5′ CGAGGCCCTTTCGTCTTCACTCTGAAACAGTGCAAAGCTA 3′ |
| | RV: |
| (SEQ ID NO: 50) | |
| | 5′ TGTTTTTTAGCGTCAGGCATCTCTAGGATTCCTCCGGTTC 3′ |

The NEB phusion DNA polymerase was used to amplify the cassette using 1 ng of pBS1C3-IHF plasmid as a template, according to the manufacturer's instructions. The PCR amplification was carried out as follows: 1 cycle of 1 min at 98° C., 5 cycles of 30 sec at 98° C., 30 sec at 60° C. and 1 min at 72° C., 30 cycles of 30 sec at 98° C., 1 min at 72° C. and 1 cycle of 5 min at 72° C. The fragment thus amplified by PCR was purified from a gel and then quantified with a Nanodrop spectrophotometer.

c) GalP transporter using the pWKS-galP plasmid as a template with the following primers:

| | FW: |
|---|---|
| (SEQ ID NO 51) | |
| | 5′ GAACCGGAGGAATCCTAGAGATGCCTGACGCTAAAAAACA 3′ |
| | RV: |
| (SEQ ID NO 52) | |
| | 5′ GCAGCCAACTCAGCTTCCTTTTAATCGTGAGCGCCTATTT 3′ |

The NEB phusion DNA polymerase was used to amplify the cassette using 1 ng of pWKS-galP plasmid as a template, according to the manufacturer's instructions. The PCR amplification was carried out as follows: 1 cycle of 1 min at 98° C., 5 cycles of 30 sec at 98° C., 30 sec at 62° C. and 1 min at 72° C., 30 cycles of 30 sec at 98° C., 1 min at 72° C. and 1 cycle of 5 min at 72° C. The fragment thus amplified by PCR was purified from a gel and then quantified with a Nanodrop spectrophotometer.

The three purified fragments were incubated together and cloning was performed using the inFusion® Takara cloning kit according to the manufacturer's instructions, which allowed to perform the ligation reaction and the transformation of chemically competent E. coli Top10 cells. Selection was performed on LB agars supplemented with 50 μg/mL of kanamycin.

The correct assembly of the plasmid was confirmed by colony PCR using the following primers:

| | FW: |
|---|---|
| (SEQ ID NO: 53) | |
| | 5′ TCTGAAACAGTGCAAAGCTA 3′ |
| | and |
| | RV: |
| (SEQ ID NO: 54 | |
| | 5′ TTAATCGTGAGCGCCTATTT 3′ |

The NEB taq DNA polymerase was used to amplify a DNA fragment between the sequence of the IHF promoter and that of the galP gene using 1 μL of a cell colony dissolved in 50 μL of sterile water according to the manufacturer's instructions. The PCR amplification was carried out as follows:

1 cycle of 5 min at 95° C., 35 cycles of 30 sec at 95° C., 30 sec at 55° C. and 1.5 min at 68° C., and 1 cycle of 5 min at 68° C.

Positive colonies were cultured in 5 ml of LB liquid medium supplemented with 50 μg/mL of kanamycin overnight at 37° C. Then, “miniprep” of the pTC-galP plasmid were carried out with the Qiagen preparation kit to recover and concentrate the pTC-galP plasmid.

### 1.5. Production of Mannobiose on a Mixture of Glycerol and Mannose.

A) The Obtained CS1 (Csoamaaamana) Strain Transformed with the Pairs of PWKS-GALP/PBAD-Teth88 (A1) or Ptc-Galp/Pbad-Teth88 (C1) Plasmids

Growth was carried out in 250 mL baffled Erlenmeyer flasks containing 50 mL of M9 medium, at 37° C. and under orbital stirring of 200 rpm. The M9 medium was supplemented with 6 g/L of glycerol and 1 g/L of mannose, 0.06 mM of IPTG and 10 mM of L-arabinose. The analysis of the culture medium supernatant was carried out by NMR as described above.

The strains were cultured after culture a supernatant sample was analyzed with to determine the concentration of β-1,2-mannobiose, according to the process described in Example 2 above, present as a function of the pWKS-galP/pBAD-Teth88 (A1) or pTC-galP/pBAD-Teth88 (C1) plasmids, present in the CS1 strain. The β-1,2-mannobiose concentration measured for the CS1 strain transformed with the pWKS-galP/pBAD-Teth88 (A1) plasmids was 0.78 mM in the supernatant of the culture medium and 0.91 mM in the supernatant for the CS1 strain transformed with the pTC-galP/pBAD-Teth88 (C1) plasmids. A determination of the conversion rate of mannose into β-1,2-mannobiose was also estimated and corresponded to 48% and 60% respectively.

This example therefore clearly demonstrates that the CS1 strain, also named CS0ΔmaaΔmanA, corresponding to the PFKA1 strain in which the maa gene and the manA gene have been inactivated advantageously allows a production of oligosaccharides. In addition, the obtained results clearly demonstrate that a strain example according to the invention advantageously and surprisingly and unexpectedly allows the excretion of oligosaccharides produced in the culture medium.

This example also demonstrates that a strain example according to the invention advantageously allows a production of oligosaccharides with high production yields.

In other words, the results clearly demonstrate that a strain example according to the invention advantageously allows the synthesis of oligosaccharides and advantageously their excretions in the culture medium.

### B) Production of β-1,2-Mannobiose in a Bioreactor

A bioreactor production of β-1,2-mannobiose by the CS1 strain with the pair of C1 plasmids was carried out. For this bioreactor experiment, the composition of the M9 salts was modified as follows: KH2PO4 3.02 g/L, NaCl 0.51 g/L, NH4C1 2.04 g/L, (NH₄)2SO₄ 5 g/L. All other components for the culture are the same as those described for the culture above. The cultures in a bioreactor were carried out in 500 ml of this M9 medium supplemented with 3 g/L of D-mannose and 20 g/L of glycerol. The analysis of the supernatant was carried out by NMR as described in Example 2. At the end of the culture, a supernatant sample was analyzed to determine the concentration of β-1,2-mannobiose, according to the process described in Example 2 above. The determined β-1,2-mannobiose concentration was 1.21 mM and the conversion rate 58%, similar to the values obtained during the aforementioned culture in Erlenmeyer flasks.

A comparison of the production of β-1,2-mannobiose in bioreactor was also carried out with the MDO, MGX, MGX1, PFKA1, CS0 Δmaa strains, expressing the Teth-1788 enzyme

Table 13 groups together the calculated production characteristics for the different strains from the concentrations of β-1,2-mannobiose and of residual mannose determined by NMR.

TABLE 13
TITERS AND YIELDS OF B-1,2-MANNOBIOSE IN THE CULTURE
SUPERNATANTS IN BIOREACTOR OF THE MDO, MGX, MGX1, PFKA1, CS0 ΔMAA,
AND CSI (ΔMAA ΔMANA) STRAINS EXPRESSING THE TETH-1788 ENZYME. THE
CONCENTRATIONS (TITER) ARE EXPRESSED IN MM AND THE YIELDS IN
PERCENTAGE OF MANNOSE CONVERTED INTO B-1,2-MANNOBIOSE.

| | | Man2 Titer | % Yield (g Man2/ |
|---|---|---|---|
| Strain | Characteristic | (mM) | g Man) |
| MDO-Teth-1788 | Control strain | ND¹ | ND |
| MGX-Teth-1788 | PTS-, no GalP; non-induced | 0.21¹ | 1.02 |
| | man B | | |
| MGX1-Teth- | PTS-with induced GalP, | 0.59¹ | 2.91 |
| 1788 | induced manB | | |
| PFKA1-Teth- | PTS-with GalP; deletion of | 1.8¹ | 9.02 |
| 1788 (CS0) | pfka; induced manB | | |
| CS0 Δmaa-Teth- | PTS-with GalP; deletion of | 1.75¹ | 8.98 |
| 1788 | pfka; induced manB. | | |
| | Deletion of Maa | | |
| CS0 maa-Teth- | PTS-with GalP; deletion of | 0.58² | 18.22 |
| 1788 | pfka; induced manB. | | |
| | Deletion of maa; glycerol | | |
| | growth | | |
| CS1 Δmaa | PTS-with GalP; deletion of | 1.21¹ | 58-72 |
| ΔmanA-Teth- | pfka; induced manB. | | |
| 1788 | Deletion of maa, Deletion | | |
| | of manA; glycerol growth | | |

In the Table
¹means growth with 10 g/L of mannose as carbon and energy source, and
²means growth with 3 g/L of mannose (conversion into β-1,2-mannobiose) and 20 g/L of glycerol (carbon and energy source).

As demonstrated above, the strain according to the invention advantageously and surprisingly allows to significantly increase the production of oligosaccharides. In addition, the results obtained clearly demonstrate that the strain according to the invention allows to significantly increase the production yields of oligosaccharides and thus to optimize/reduce the production costs.

This example therefore clearly demonstrates that the strain that a strain example according to the invention advantageously allows the production of oligosaccharides. In addition, the obtained results clearly demonstrate that a strain example according to the invention advantageously and surprisingly and unexpectedly allows the excretion of oligosaccharides produced in the culture medium.

This example also demonstrates that a strain example according to the invention advantageously allows a production of oligosaccharides with high production yields.

In other words, the results clearly demonstrate that strain examples according to the invention advantageously allow the synthesis of oligosaccharides and advantageously their excretions in the culture medium.

### Example 4: Manufacture and Characterization of the Strain Filed with the Cncm Under Number Cncm I-5682

From the strain described in Example 3, the following modifications were provided to obtain further improved yields for the conversion of mannose into mannobiose.

In the example below, the strain filed with the CNCM (Collection Nationale de Culture de Microorganismes, Institut Pasteur, 25 rue du Docteur Roux, 75724 Paris Cedex 15, France), under number CNCM I-5682 is also designated CS1 IG or OLI-CS1 IG or CS1 IG-galP strain.

The GalP gene is weakly expressed due to two repressors gaIR and galS. The inventors have surprisingly highlighted that it is possible to avoid the use of plasmids for the expression of the GalP gene and to stabilize the bacterial frame by increasing the expression of the GalP gene via the modification of its promoter. The region upstream of the gene (400 base pairs), which is recognized by the repressors, is substituted with a DNA fragment carrying the IHF promoter of E. coli (Zhou, K., Zhou, L., Lim, Q. 'En, Zou, R., Stephanopoulos, G., and Too, H.-P. (2011) Novel reference genes for quantifying transcriptional responses of Escherichia coli to protein overexpression by quantitative PCR. BMC Mol Biol 12: 18 [65]).

In the present, the substitution of a DNA sequence can be carried out by any suitable process known to the person skilled in the art. It may be, for example, the Datsenko and Wanner protocol (Kirill A. Datsenko, Barry L. Wanner. One-step inactivation of chromosomal genes in Escherichia coli K-12 using PCR products. Proceedings of the National Academy of Sciences June 2000, 97 (12) 6640-6645; DOI: 10.1073/pnas.120163297 [62]).

### 1. Construction of the Replacement Fragment Carrying the Ihf Promoter

The first step is the assembly of the fragment intended to be integrated to the genome. The sample comprises the kanamycin-resistance gene (1500 bp) and the IHF promoter. These two fragments were individually amplified and then combined together to form the replacement fragment of 2300 bp.

A DNA fragment containing the kanamycin-resistance gene and flanked by Flippase Recognition Target (FRT) sites was amplified with the following primers:

fw:
(SEQ ID NO: 56)
5′-GCACAATAACATCATTCTTCCTGATCACGTTTCACCGCAGATTATCA
TCAAGATTGCAGCATTACACGTCTT 3′
and
rev:
(SEQ ID NO: 57)
5′-TGTGGGTTTCCGCGAACTGCTGCCACGGGTTAGCTTTGCACTGTTTC
AGATCCATATGAATATCCTCCTTAGTTCCT 3′

The fw primer has 50 bp homology with the region immediately upstream of the sequence to be deleted. The rev primer has 20 bp homology with the 5′ end of the IHF promoter. The Phusion DNA polymerase (NEB) was used to amplify the cassette using 1 ng of pKD4 plasmid as a template, according to the manufacturer's protocol. The PCR cycles are carried out as follows:

1 cycle of 1 min at 98° C., 5 cycles of 30 seconds at 98° C., 30 seconds at 63° C. and 1 min at 72° C., 30 cycles of 30 seconds at 98° C., 1 min at 72° C., and 1 cycle of 5 min at 72° C.

The promoter fragment was amplified with the following primers:

fw:
(SEQ ID NO 58)
5′-AGGAACTAAGGAGGATATTCATATGGATCTGAAACAGTGCAAAGCTA
ACCCGTGGCAGCAGTTCGCGGAAACCCACA 3′
and
rev:
(SEQ ID NO 59)
5′-AAAAACGTCATTGCCTTGTTTGACCGCCCCTGTTTTTTAGCGTCAGG
CATCTCTAGGATTCCTCCGGTTCCT 3′

The FW primer (SEQ ID NO 58) has a 20 bp homology with the 3′ end of the kan cassette sequence. The rev primer (SEQ ID NO 59) has 50 bp homology with the genome region immediately upstream of the sequence to be deleted.

The phusion DNA polymerase (NEB) is used to amplify the cassette using 1 ng of pKD4 plasmid as template, according to the manufacturer's protocol. The PCR cycles are carried out as follows:

1 cycle of 1 min at 98° C., 5 cycles of 30 seconds at 98° C., 30 seconds at 64° C. and 30 at 72° C., 30 cycles of 30 seconds at 98° C., 30 seconds at 72° C., and 1 cycle of 5 min at 72° C.

To generate the final fragment a third PCR is carried out with the with the following primers:

| | fw: |
|---|---|
| (SEQ ID NO 60) | |
| | 5′ GCACAATAACATCATTCTTCCTGATCACG 3′ |
| | and |
| | rev: |
| (SEQ ID NO 61) | |
| | 5′ AAAAACGTCATTGCCTTGTTTGACCG 3′ |

As template 1 μL of PCR1 and 1 μL of PCR2 are used. The PCR cycles are carried out as follows: 1 cycle of 1 min at 98° C., 35 cycles of 30 seconds at 98° C., 30 seconds at 65° C. and 1 min 30 seconds at 72° C., and 1 cycle 5 min at 72° C.

The PCR amplicon is gel purified and quantified. This fragment is used for the substitution of the promoter of the GalP gene.

### 2. Homologous Recombination

The CS1 strain was transformed with the pKD46 plasmid at 30° C. on LB agar agar supplemented with ampicillin at 50 μg/mL. The strain transformed carrying the pKD46 plasmid was cultured in 50 ml of LB medium comprising ampicillin and L-arabinose (0.2% w/v) at a temperature of 30° C. for an OD600 of 0.6 then concentrated 100 times to make it electrocompetent, washed three times with cold water containing 10% glycerol. Electroporation was carried out using a Cell-Porator with a voltage amplifier and 0.1 cm chambers according to the manufacturer's instructions, using 50 μL of cells and 200 ng of the PCR fragments obtained above.

One milliliter of SOC medium (2% tryptone; 0.5% yeast extract; 10 mM NaCl, 2.5 mM KCl, 10 mM MgCl₂, 10 mM MgSO₄, and 20 mM glucose) was added to the cells having undergone this treatment, incubated for 2 hours at 37° C., then spread on a LB agar comprising kanamycin to select the strains. The introduction of the IHF promoter upstream of the GalP gene was confirmed by PCR on a colony using the following primers:

| | fw: |
|---|---|
| (SEQ ID NO: 62) | |
| | 5′ CAGCCTGTCTGTTCGTGCGAAAGA 3′ |
| | and |
| | rev: |
| (SEQ ID NO: 63) | |
| | 5′ TCAGAGAGGTACAGCGGTG 3′ |

The NEB taq DNA polymerase was used to amplify an IHF promoter and galP gene fragment using 1 μL of a cell colony dissolved in 50 μL of sterile water according to the manufacturer's instructions. The PCR amplification was carried out as follows: 1 cycle of 5 min at 95° C., 35 cycles of 30 sec at 95° C., 30 sec at 55° C. and 1.5 min at 68° C., and 1 cycle of 5 min at 68° C. Once the colonies having lost the gene were identified, the elimination of the kanamycin resistance cassette was carried out by transforming, one of the positive colonies with the pCP20 plasmid. The pCP20 plasmid is an ampicillin-resistance plasmid with temperature sensitive replication and an induction of the FLP synthesis by heat shock. The Kanamycin-resistant (KmR) mutants were transformed with the pCP20 plasmid, and the ampicillin-resistant transformants were selected at 30° C. Transformed strains were isolated on LB agar non-selectively at 43° C. and then tested for the loss of all antibiotic resistances.

### 3. Cloning of the Gene Encoding a β-1,2-Mannobiose Phosphorylase Enzyme in the Pbad-Hisa Plasmid (Primers and Plasmid Sequence)

The gene encoding the Lin0857 protein of the Listeria innocua (Lin0857, Genbank accession number CAC96089) belonging to the GH130 family of the CAZy classification (http://www.cazy.org/) (Lombard, V., Golaconda Ramulu, H., Drula, E., Coutinho, P. M., Henrissat, B., 2014. The carbohydrate-active enzymes database (CAZy) in 2013. Nucleic Acids Res. 42, D490-D495 [30]) has been synthesized and cloned into the pET28a vector by the TWIST Bioscience company (San Francisco, Calif. 94080, USA) with an optimization of the use of codons for an expression of the gene in E. coli. The gene encoding Lin0857 and the pBAD-Hisa plasmid were gel purified after digestion with the NcoI-HF and XhoI restriction enzymes according to the protocol of the QIAEX II Gel Extraction Kit (Hilden, Germany). The ligation of the pBAD vector and the gene encoding Lin0857 (insert) was carried out in the presence of 10 μL of vector at 8 ng/μL, 7 μL of insert at 7 ng/μL, 2 μL of DNA ligase buffer 10× and 1.5 μL of 400,000 U/mL T4 DNA ligase (New England Biolabs, Ipswich, Mass., USA). The reaction is incubated for 1 hour at room temperature then 10 min at 65° C. and 10 min in ice and 6 μL of this reaction are deposited in 50 μL of competent commercial Stellar cells (Clontech Laboratories, Mountain View, USA) for transformation. following the classic protocol for transforming chemo-competent cells (https://www.addgene.org/protocols/bacterial-transformation/[50]). The construction was monitored by sequencing from the Eurofins genomics company (Luxembourg) using the following primers: pBAD-fw: 5′-ATGCCATAGCATTTTTATCC-3′ (SEQ ID NO: 64) and pBAD-rev: 5′-GATTTAATCTGTATCAGG-3′ (SEQ ID NO: 65).

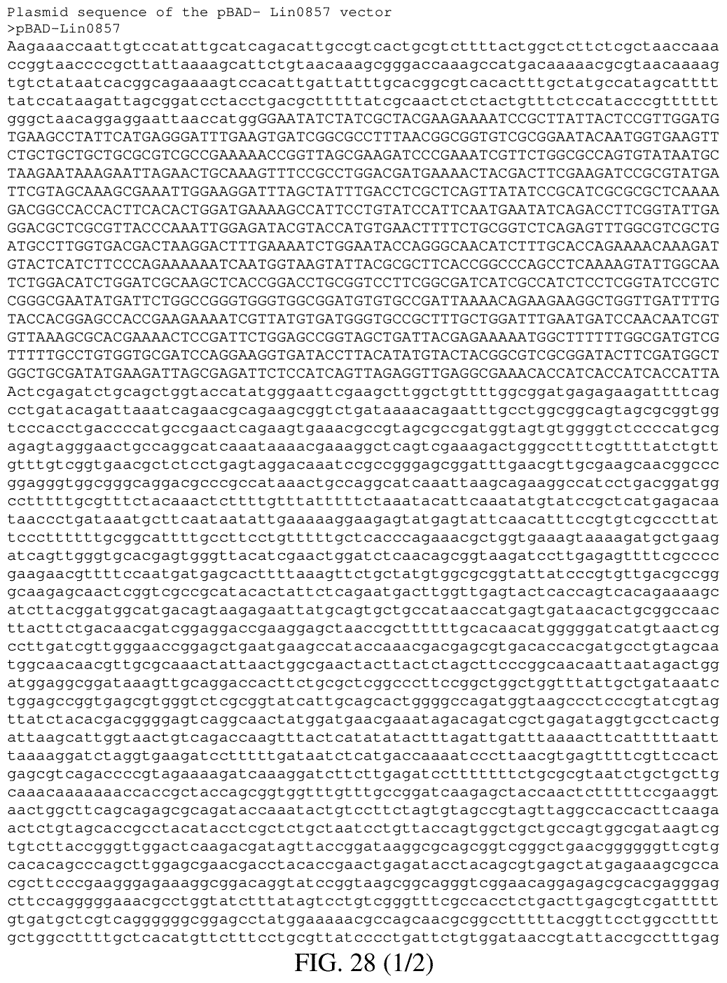

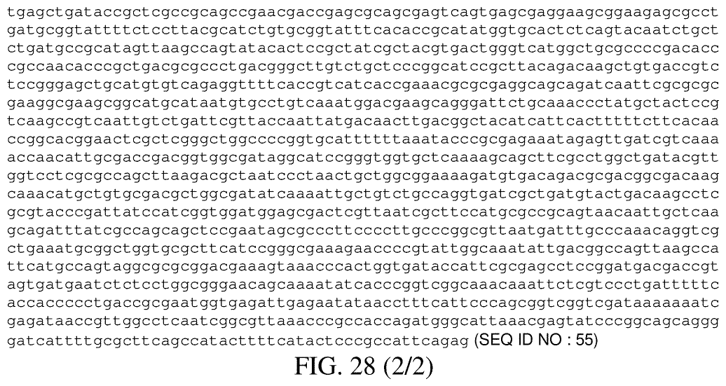

The obtained plasmid sequence corresponds to sequence SED ID NO: 55 represented in FIG. 28.

The CS0, CS1 and CS1 IG strains were transformed according to the classic protocol for transforming chemo-competent cells (https://www.addgene.org/protocols/bacterial-transformation/[50]) with the pWKS-galP, pBAD-Teth1788 and/or pBAD-Lin0857 plasmid as indicated in Table (14) below.

TABLE 14
STRAINS AND CHARACTERISTICS

| Identifier | Strain | Characteristics |
|---|---|---|
| A | CS0 | MDO ptsG manXYZ pfkA |
| B | CS0-galβ-Teth1788 | CS0 pWKS-galP; pBAD-Teth1788 |
| C | CS1-galβ-Teth1788 | CS0 ΔmaaΔmanA; pWKS-galP; pBAD- |
| | | Teth1788 |
| D | CS1 IG-Teth1788 | CS1 galP: Pihf; pBAD-Teth1788 |
| E | CS1-galβ-Lin0857 | CS0 ΔmaaΔmanA; pWKS-galP; pBAD- |
| | | Lin0857 |
| F | CS1 IG-galβ-Lin0857 | CS1 galP: Pihf pWKS-galP; pBAD- |
| | | Lin0857 |

In the table above, “Pihf” means IHF promoter

These 6 strains were placed in precultures overnight (10 to 15 hours) in LB selective medium to inoculate a second preculture (15 to 20 hours) in selective M9 medium supplemented with glycerol (10 g/L) and d-Mannose (5 g/L) as carbon source. The 6 cultures of 50 mL were inoculated with the corresponding precultures to obtain an OD₆₀₀=0.15 at t=0 h in selective M9 medium supplemented with glycerol (10 g/L) and d-Mannose at 5 g/L as carbon source in 500 mL baffled Erlenmeyer flasks. The Erlenmeyer flasks were incubated for 47 h at 37° C. with orbital stirring of 220 revolutions per minute (rpm). The addition of 1-arabinose at 0.1% in the culture medium when the OD₆₀₀ is ≈0.4 allows the expression of the glycoside phosphorylase gene present in the pBAD (Teth1788 or Lin0857) plasmid and the final addition of IPTG at 60 μM allows the expression of the GalP gene in cultures containing the pWKS-galP plasmid.

FIG. 27 shows the growth curves of the different strain cultures mentioned in Table 14 above. The growth determination was performed by measuring the optical density at 600 nm (ordinate) (OD₆₀₀) as a function of time in hours (abscissa).

Supernatant samples were withdrawn after 31 hours of culture in the 6 cultures and diluted 10 times in ultra pure water to be analyzed by HPAEC-PAD according to the protocol previously described (see section 2.3 of example 2). The titers or concentration of β-1,2-mannobiose present in the supernatants and the conversion yields of the consumed mannose converted into mannobiose were calculated and presented in Table 15 below. The concentrations (titer) are expressed in mM and the yields in percentage of mannose converted into β-1,2-mannobiose.

TABLE 15
PERCENTAGE OF CONSUMED MANNOSE,
YIELDS AND CONCENTRATION
OF B-1,2-MANNOBIOSE (MANNOBIOSE) IN
THE SUPERNATANTS AFTER 31 HOURS
OF CULTURES OF THE 6 STRAINS.

| | | | Mannose converted | Mannobiose |
|---|---|---|---|---|
| | | Consumed | into mannobiose | concentration |
| | Strain | mannose (%) | (%) (g Man2/g Man) | (mM) |
| A | CS0 | 68.5 | 0 | 0 |
| B | CS0-galβ- | 91.6 | 9 | 1.22 |
| | Teth1788 | | | |
| C | CS1-galβ- | 21.7 | 71 | 2.24 |
| | Teth1788 | | | |
| D | CS1 IG- | 14.1 | 49 | 1.01 |
| | Teth1788 | | | |
| E | CS1-galβ- | 19.6 | 51 | 1.47 |
| | Lin0857 | | | |
| F | CS1 IG-galβ- | 21.0 | 54 | 1.67 |
| | Lin0857 | | | |

As demonstrated in Table 15 above, the results demonstrate that the conversion yield is markedly improved with the CS1 and CS1 IG strains (>49%). This therefore clearly demonstrates that mannose is used mainly as substrate for the synthesis of mannobiose and glycerol as carbon source for the growth. These results also demonstrate that the replacement of the native promoter of galP by a constitutive strong promoter on the genome allows to obtain a high conversion yield (49%), which presents a significant advantage for the production process because this can advantageously allow to not use the plasmid containing the GalP gene and therefore the antibiotic necessary for its retention in the strain. As demonstrated, the strain comprising a constitutive promoter upstream of the GalP gene, namely the CS1 IG strain (condition F) also allows to obtain a better conversion yield and a higher mannobiose titer in the presence of the pWKS-GalP vector compared to the CS1 strain (condition E) which does not contain this promoter modification.

As demonstrated above, the strains according to the invention advantageously and surprisingly allow to significantly increase the production of oligosaccharides. In addition, the results obtained clearly demonstrate that the strain according to the invention allows to significantly increase the production yields of oligosaccharides and thus to optimize/reduce the production costs.

This example therefore clearly demonstrates that the strain that a strain example according to the invention advantageously allows the production of oligosaccharides. In addition, the obtained results clearly demonstrate that a strain example according to the invention advantageously and surprisingly and unexpectedly allows the excretion of oligosaccharides produced in the culture medium.

This example also demonstrates that a strain example according to the invention advantageously allows a production of oligosaccharides with high production yields.

### LISTS OF REFERENCES

[1] Faille, C., Michalski, J. C., Strecker, G., Mackenzie, D. W., Camus, D., Poulain, D., 1990. Immunoreactivity of neoglycolipids constructed from oligomannosidic residues of the Candida albicans cell wall. Infect. Immun. 58, 3537-3544.
[2] Fierfort, N., Samain, E., 2008. Genetic engineering of Escherichia coli for the economicFaille, C., Michalski, J. C., Strecker, G., Mackenzie, D. W., Camus, D., Poulain, D., 1990. Immunoreactivity of neoglycolipids constructed from oligomannosidic residues of the Candida albicans cell wall. Infect. Immun. 58, 3537-3544.
[3] Fierfort, N., Samain, E., 2008. Genetic engineering of Escherichia coli for the economical production of sialylated oligosaccharides. J. Biotechnol. 134, 261-265.
[4] Ishii, N., Nakahigashi, K., Baba, T., Robert, M., Soga, T., Kanai, A., Hirasawa, T., Naba, M., Hirai, K., Hoque, A., Ho, P. Y., Kakazu, Y., Sugawara, K., Igarashi, S., Harada, S., Masuda, T., Sugiyama, N., Togashi, T., Hasegawa, M., Takai, Y., Yugi, K., Arakawa, K., Iwata, N., Toya, Y., Nakayama, Y., Nishioka, T., Shimizu, K., Mori, H., Tomita, M., 2007. Multiple High-Throughput Analyses Monitor the Response of E. coli to Perturbations. Science 316, 593-597.
[5] Kiefer, P., Nicolas, C., Letisse, F., Portais, J.-C., 2007. Determination of carbon labeling distribution of intracellular metabolites from single fragment ions by ion chromatography tandem mass spectrometry. Anal. Biochem. 360, 182-188.
[6] Liang, Q., Zhang, F., Li, Y., Zhang, X., Li, J., Yang, P., Qi, Q., 2015. Comparison of individual component deletions in a glucose-specific phosphotransferase system revealed their different applications. Sci. Rep. 5, 13200.
[7] Link, A. J., Phillips, D., Church, G. M., 1997. Methods for generating precise deletions and insertions in the genome of wild-type Escherichia coli: application to open reading frame characterization. J. Bacteriol. 179, 6228-6237. https://doi.org/10.1128/jb.179.20.6228-6237.1997.
[8] Lombard, V., Golaconda Ramulu, H., Drula, E., Coutinho, P. M., Henrissat, B., 2014. The carbohydrate-active enzymes database (CAZy) in 2013. Nucleic Acids Res. 42, D490-D495.
[9] Mashego, M. R., Wu, L., Van Dam, J. C., Ras, C., Vinke, J. L., Van Winden, W. A., Van Gulik, W. M., Heijnen, J. J., 2004. MIRACLE: mass isotopomer ratio analysis of U-13C-labeled extracts. A new method for accurate quantification of changes in concentrations of intracellular metabolites. Biotechnol. Bioeng. 85, 620-628.
[10] Rong Fu Wang, Kushner, S. R., 1991. Construction of versatile low-copy-number vectors for cloning, sequencing and gene expression in Escherichia coli. Gene 100, 195-199.
[11] Shibata, N., Hisamichi, K., Kikuchi, T., Kobayashi, H., Okawa, Y., Suzuki, S., 1992. Sequential nuclear magnetic resonance assignment of beta.-1,2-linked mannooligosaccharides isolated from the phosphomannan of the pathogenic yeast Candida albicans NIH B-792 strain. Biochemistry 31.
[12] Wu, L., Mashego, M. R., van Dam, J. C., Proell, A. M., Vinke, J. L., Ras, C., van Winden, W. A., van Gulik, W. M., Heijnen, J. J., 2005. Quantitative analysis of the microbial metabolome by isotope dilution mass spectrometry using uniformly 13C-labeled cell extracts as internal standards. Anal. Biochem. 336, 164-171.
[13] Combes, Monsan, 2009. Biocatalyse ou catalyse enzymatique. Procédés chimie-bio-agro I Chimie verte, Techniques de l'ingénieur 1-18.
[14] Rudroff, F., Mihovilovic, M. D., Gröger, H., Snajdrova, R., Iding, H., Bornscheuer, U. T., 2018. Opportunities and challenges for combining chemo- and biocatalysis. Nature Catalysis 1, 12-22. https://doi.org/10.1038/s41929-017-0010-4.
[15] Anastas et Warner, 2000 Anastas, P. T., Warner, J. C., 2000. Green chemistry: theory and practice, 1. paperback. ed. Oxford Univ. Press, Oxford. Imeson, 2009 Hydrocolloids as thickening and gelling agents in food: a critical review, J Food Sci Technol. 2010 December; 47(6): 587-597.
[16] Tathiana Souza Martins Meyer, Angelo Samir Melim Miguel, Daniel Ernesto Rodríguez Fernández and Gisela Maria Dellamora Ortiz “Biotechnological Production of Oligosaccharides—Applications in the Food Industry” Oct. 22nd 2015.
[17] Rudroff et al., Rudroff, F., Mihovilovic, M. D., Gröger, H., Snajdrova, R., Iding, H., Bornscheuer, U. T., 2018. Opportunities and challenges for combining chemo- and biocatalysis. Nature Catalysis 1, 12-22. 8 Jan. 2018.
[18] Ager, D., 2012. Final Analysis: Biological or Chemical Catalysis? Platinum Metals Review. Volume 56, Number 2, April 2012, pp. 140-142(3).
[19] Reetz, M. T., 2013. Biocatalysis in Organic Chemistry and Biotechnology: Past, Present, and Future. Journal of the American Chemical Society 135, 12480-12496.
[20] https://doi.org/10.1021/ja405051f.
[21] Puchart, V., 2015. Glycoside phosphorylases: Structure, catalytic properties and biotechnological potential. Biotechnology Advances 33, 261-276.
[22] Cobucci-Ponzano, B., Moracci, M., 2012. Glycosynthases as tools for the production of glycan analogs of natural products. Natural Product Reports 29, 697. March 2012.
[23] WO0104341, Samain & Priem (2001) Procédé production d'oligosaccharides.
[24] WO2008040717, Samain (2008) High yield production of sialic acid (Neu5ac) by fermentation.
[25] Murray E. J. (éd.), Methods in Molecular Biology, Vol. 7, Gene Transfer and Expression Protocols, Humana Press (1991).
[26] Johnston, Biolistic transformation: microbes to mice. Nature, 346: 776-777 (1990).
[27] Brash et al., Strontium phosphate transfection of human cells in primary culture: stable expression of the simian virus 40 large-T-antigen gene in primary human bronchial epithelial cells. Mol. Cell Biol. 7: 2031-2034 (1987).
[28] Quanfeng Liang, 1 Fengyu Zhang, 1 Yikui Li, 1 Xu Zhang, 1 Jiaojiao Li, 1 Peng Yang, 1 and Qingsheng Qia, 1 Comparison of individual component deletions in a glucose-specific phosphotransferase system revealed their different applications Sci Rep. 2015; 5: 13200. Published online 2015 Aug. 19.
[29] Sonja Steinsiek and Katja Bettenbrock Glucose Transport in Escherichia coli Mutant Strains with Defects in Sugar Transport Systems, J Bacteriol. 2012 November; 194(21): 5897-5908.
[30] Lombard, V., Golaconda Ramulu, H., Drula, E., Coutinho, P. M., Henrissat, B., 2014. The carbohydrate-active enzymes database (CAZy) in 2013. Nucleic Acids Res. 42, D490-D495.
[31] https://www.addgene.org/mol-bio-reference/cloning.
[32] Erju Tang, Xialin Shen, Jia Wang, Xinxiao Sun, Qipeng Yuan, “Synergetic utilization of glucose and glycerol for efficient myo-inositol biosynthesis” Biotechnology and Bioengineering 2020.
[33] Constantin Ruprecht, Friedericke Bönisch, Nele Ilmbergera, Tanja V. Heyera, Erhard T. K. Haupt, Wolfgang R. Streit, Ulrich Rabausch “High level production of flavonoid rhamnosides by metagenome-derived Glycosyltransferase C in Escherichia coli utilizing dextrins of starch as a single carbon source”, Metabolic Engineering Volume 55, September 2019, Pages 212-219.
[34] Tatiana Antoine, Alain Heyraud Dr., Claude Bosso Dr., Eric Samain Dr “Highly Efficient Biosynthesis of the Oligosaccharide Moiety of the GD3 Ganglioside by Using Metabolically Engineered Escherichia coli”, Angewandte Chemie, Volume117, Issue9, Feb. 18, 2005 Pages 1374-1376.
[35] https://france.promega.com/products/vectors/protein-expression-vectors.
[36]https://www.thermofisher.com/search/browse/category/fr/fr/85801/Bacterial %20Expres sion %20Vectors
[37] WO 83/004261.
[38]https://www.embl.de/pepcore/pepcore_services/strains_vectors/vectors/bacterial_expressio n_vectors/popupbacterial_expression_vectors/index.html.
[39] http://babel.ucmp.umu.se/cpep/web_content/Pages/CPEP_09_vectors.html.
[40]https://www.embl.de/pepcore/pepcore_services/strains_vectors/vectors/gateway_vector s/index.htm.
[41] http://parts.igem.org/Promoters/Catalog/Constitutive.
[42] https://biocyc.org/group?id=:ALL-PROMOTERS&orgid=ECOLI.
[43] http://parts.igem.org/Promoters/Catalog.
[44] Israel Pedruzzi, Eduardo Borges da Silva, Alírio E Rodrigues “Selection of resins, equilibrium and sorption kinetics of lactobionic acid, fructose, lactose and sorbitol” Separation and Purification Technology 2008 Volume 63, Issue 3, 3 Nov. 2008, Pages 600-611.
[45] Clarisse Nobre, José A. Teixeira & LígiaR. Rodrigues (2015) “New Trends and Technological Challenges in the Industrial Production and Purification of Fructo-oligosaccharides”, Critical Reviews in Food Science and Nutrition, 55:10, 1444-1455, October 2013.
[46] https://www.novasep.com/technologies/industrial-technologies-for-evaporation-concentration-and-crystallization.html.
[47] https://international.neb.com/protocols/2014/05/07/double-digest-protocol-with-standard-restriction-enzymes.
[48] https://international.neb.com/protocols/0001/01/01/pcr-protocol-m0530.
[49] https://international.neb.com/protocols/0001/01/01/dna-ligation-with-t4-dna-ligase-m0202.
[50] https://www.addgene.org/protocols/bacterial-transformation/.
[51] http://tools.thermofisher.com/content/sfs/manuals/pentr_dtopo_man.pdf.
[52] Karen L. Elbing Roger Brent, Recipes and Tools for Culture of Escherichia coli, Current Tools, 9 Nov. 2018.
[53] Quantification of Sugar Compounds and Uronic Acids in Enzymatic Hydrolysates of Lignocellulose Using High-Performance Anion Exchange Chromatography with Pulsed Amperometric Detection Energy Fuels 2012, 26, 5, 2942-2947.
[54] WO2007101862, Samain (2007) Method of producing sialylated oligosaccharides.
[55] WO2007023348, SAMAIN et al. (2007) Production of globoside oligosaccharides using metabolically engineered microorganisms.
[56] Fort, S., Birikaki, L., Dubois, M. P., Antoine, T., Samain, E., & Driguez, H. (2005). Biosynthesis of conjugatable saccharidic moieties of GM 2 and GM 3 gangliosides by engineered E. coli. Chemical communications, (20), 2558-2560.
[57] Fierfort, N., & Samain, E. (2008). Genetic engineering of Escherichia coli for the economical production of sialylated oligosaccharides. Journal of biotechnology, 134β-4), 261-265.
[58] https://www.takarabio.com/learning-centers/cloning/in-fusion-cloning-overview.
[59] Nord LI, Vaag P, Duus JØ Quantification of organic and amino acids in beer by ¹H NMR spectroscopy Anal Chem. 2004 Aug. 15; 76(16):4790-8.
[60] Gloriadel CampoIñakiBerregiRaúlCaracenaJ. IgnacioSantos “Quantitative analysis of malic and citric acids in fruit juices using proton nuclear magnetic resonance spectroscopy.
[61] Chiku K, Nihira T, Suzuki E, Nishimoto M, Kitaoka M, et al. (2014) Discovery of Two b-1,2Mannoside Phosphorylases Showing Different Chain-Length Specificities from Thermoanaerobacter sp. X-514. PLoS ONE 9(12): e114882. doi:10.1371/journal.pone.0114882.
[62] Kirill A. Datsenko, Barry L. Wanner. One-step inactivation of chromosomal genes in Escherichia coli K-12 using PCR products. Proceedings of the National Academy of Sciences June 2000, 97 (12) 6640-6645; DOI: 10.1073/pnas.120163297
[63] Leila Lo Leggio, Florence Dal Degan, Peter Poulsen, Søren Møller Andersen, and Sine Larsen. The Structure and Specificity of Escherichia coli Maltose Acetyltransferase Give New Insight into the LacA Family of Acyltransferases. Biochemistry 2003, 42, 18, 5225-5235; DOI: 10.1021/bi0271446
[64] M Freundlich, N Ramani, E Mathew, A Sirko, P Tsui, “The role of integration host factor in gene expression in Escherichia coli” Mol Microbiol. 1992 September; 6(18):2557-63
[65] Zhou, K., Zhou, L., Lim, Q. 'En, Zou, R., Stephanopoulos, G., and Too, H.-P. (2011) Novel reference genes for quantifying transcriptional responses of Escherichia coli to protein overexpression by quantitative PCR. BMC Mol Biol 12: 18

1. An Escherichia coli strain whose recA1, gyrA96, thi-1, glnV44 relA1 hsdR17, endA1, lacZ, nanKETA, lacA, melA, wcaJ, mdoH, ptsG, manX, manY, and pfkA are inactivated.

2. The strain according to claim 1, deposited with the CNCM (Collection Nationale de Culture de Microorganismes, Institut Pasteur, 25 rue du Docteur Roux, 75724 Paris Cedex 15, France), under number CNCM I-5499.

3. The strain according to claim 1 further comprising a Δmaa and a ΔmanA mutation.

4. The strain according to claim 3, deposited with the CNCM (Collection Nationale de Culture de Microorganismes, Institut Pasteur, 25 rue du Docteur Roux, 75724 Paris Cedex 15, France), under number CNCM I-5681.

5. The strain according to claim 3, in which a promoter of the GalP gene is an HIF promoter.

6. The strain according to claim 1 further comprising an expression vector of a glycoside-phosphorylase selected from a β-glycoside- or a α-glycoside-phosphorylases.

7. The strain according to claim 6, in which the β-glycoside—or the α-glycoside-phosphorylase is selected from the group comprising α-1, 3-glucopyranosyl-L-rhamnose-phosphorylases, α-1, 2-glucosyl-glycerol-phosphorylases, trehalose phosphorylases, laminaribiose-phosphorylases, D-galactosyl-β-1, 4-L-rhamnose phosphorylases, β-1, 4-mannosyl-glucose phosphorylases, β-1, 2-oligomannan phosphorylases-1, 2-mannobiose phosphorylases, β-1, 4-mannopyranosyl-[N-glycan]-phosphorylases/β-1, 4-mannopyranosyl-chitobiose-phosphorylases, β-1, 4-mannooligosaccharide phosphorylases, β-1, 3-mannooligosaccharide phosphorylases, β-1, 3-mannosyl-glucose phosphorylases, and β-1, 4-mannosyl-glucuronate phosphorylases.

8. The strain of claim 1 for in vitro use for producing oligosaccharides or in a process for producing oligosaccharides.

9. An in vitro or in cellulo method for producing oligosaccharides comprising the steps of:
a) transforming the strain according to claim 1 with an expression vector of an enzyme,
b) culturing of the transformed strain obtained in step a) or culturing a strain according to claim 1 in a culture medium, and
c) recovering of the produced oligosaccharides.

10. The method according to claim 9, wherein the recovering of the produced oligosaccharides is performed in the culture medium.

11. The method of claim 9, wherein the method at step a) comprises step a′) of transforming said strain with an expression vector of at least one transport protein or permease.

12. The method of claim 11, wherein the culture medium comprises at least one non-phosphorylated carbohydrate selected from the group comprising N-acetyl-(xD-glucosamine, galactose, glucose, lactose, glycerol, mannose, N-acetyl-glucosamine β-1,4-N-acetyl-glucosamine, and fucose.

13. The method according to claim 9, wherein the enzyme is selected from a β-glycoside- or an α-glycoside-phosphorylase.

14. The method according to claim 13, wherein the enzyme is selected from α-1, 3-glucopyranosyl-L-rhamnose-phosphorylases, α-1,2-glucosyl-glycerol-phosphorylases, trehalose-phosphorylases, laminaribiose-phosphorylases, D-galactosyl-β-1,4-L-rhamnose phosphorylases, β-1,4-mannosyl-glucose-phosphorylases, β-1,2-oligomannan phosphorylases, β-1, 2-mannobiose-phosphorylases, β-1,4-mannopyranosyl-[N-glycan]-phosphorylases, β-1, 4-mannopyranosyl-chitobiose-phosphorylases, β-1, 4-mannooligosaccharide-phosphorylases, β-1, 3-mannooligosaccharide phosphorylases, β-1,3-mannosyl-glucose phosphorylases, and β-1,4-mannosyl-glucuronate phosphorylases.

15. The method according to claim 9, wherein the medium is chosen from a rich medium, LB (Lysogeny broth), Superbroth, TB (Terrific Broth), YPD (Yeast Extract-Peptone Dextrose) medium, a minimum medium, M9 medium or M63 medium supplemented with a carbon source, a selective medium, and YNB medium (Yeast Nitrogen Base)

16. The strain according to claim 3 further comprising an expression vector of a glycoside-phosphorylase selected from β-glycoside- or α-glycoside-phosphorylases.

17. The strain according to claim 16, in which the β-glycoside- or α-glycoside-phosphorylase is chosen from α-1, 3-glucopyranosyl-L-rhamnose-phosphorylases, α-1, 2-glucosyl-glycerol-phosphorylases, trehalose phosphorylases, laminaribiose-phosphorylases, D-galactosyl-β-1, 4-L-rhamnose phosphorylases, β-1, 4-mannosyl-glucose phosphorylases, -1, 2-oligomannan phosphorylases, β-1, 2-mannobiose phosphorylases, β-1, 4-mannopyranosyl-[N-glycan]-phosphorylases, β-1, 4-mannopyranosyl-chitobiose-phosphorylases, β-1, 4-mannooligosaccharide phosphorylases, β-1, 3-mannooligosaccharide phosphorylases, β-1, 3-mannosyl-glucose phosphorylases, and β-1, 4-mannosyl-glucuronate phosphorylases.

18. A method for in vitro or in cellulo production of oligosaccharides comprising the steps of:
a) transforming the strain according to claim 3 with a vector for expressing an enzyme,
b) culturing of the transformed strain obtained in step a) or culturing a strain according to claim 3 in a culture medium, and
c) recovering the oligosaccharides produced.

19. The method according to claim 18, wherein the enzyme is selected from a β-glycoside- or an α-glycoside-phosphorylases.

20. The method of claim 19, wherein the enzyme is selected from α-1,3-glucopyranosyl-L-rhamnose-phosphorylases, α-1,2-glucosyl-glycerol-phosphorylases, trehalose phosphorylases, laminaribiose-phosphorylases, D-galactosyl-β-1,4-L-rhamnose phosphorylases, β-1,4-mannosyl-glucose phosphorylases, -1,2-oligomannan phosphorylases, β-1,2-mannobiose phosphorylases, β-1,4-mannopyranosyl-[N-glycan]-phosphorylases, β-1,4-mannopyranosyl-chitobiose-phosphorylases, β-1,4-mannooligosaccharide phosphorylases, β-1,3-mannooligosaccharide phosphorylases, β-1,3-mannosyl-glucose phosphorylases, and β-1,4-mannosyl-glucuronate phosphorylases.


SEQUENCE LISTING (66 sequences)

SEQ ID NO: 1 - DNA, 29 bp, Artificial Sequence
  amorce PL1
ctcgagaggcgatatttttccctgtatcc

SEQ ID NO: 2 - DNA, 32 bp, Artificial Sequence
  amorce PL2
ggtttgcgtattattcagtttcaacgcgttcg

SEQ ID NO: 3 - DNA, 35 bp, Artificial Sequence
  amorce PL5
ctggagctcagaggaataatacatgtcaaaagtcg

SEQ ID NO: 4 - DNA, 29 bp, Artificial Sequence
  amorce PL6
ctcgagacaacagcgataatcacatcacc

SEQ ID NO: 5 - DNA, 32 bp, Artificial Sequence
  amorce PL3
gttgaaactgaataatacgcaaaccgcctctc

SEQ ID NO: 6 - DNA, 34 bp, Artificial Sequence
  amorce PL4
atgtattattcctctgagctccagcttttgttcc

SEQ ID NO: 7 - DNA, 19 bp, Artificial Sequence
  amorce PL7
tcgaggtcattagccacca

SEQ ID NO: 8 - DNA, 33 bp, Artificial Sequence
  amorce
ctaggatccgccgaaaattgggcggtgaataac

SEQ ID NO: 9 - DNA, 30 bp, Artificial Sequence
  amorce
gtaaagcttctggtaagcagcccagagaag

SEQ ID NO: 10 - DNA, 31 bp, Artificial Sequence
  amorce
ctcaagcttgcgccgatcctgtacatcatcc

SEQ ID NO: 11 - DNA, 34 bp, Artificial Sequence
  amorce
cgtgtcgaccgttaatcctaatctgccacgcacc

SEQ ID NO: 12 - DNA, 24 bp, Artificial Sequence
  amorce
actcgagaatggcgatgaagagag

SEQ ID NO: 13 - DNA, 19 bp, Artificial Sequence
  amorce
ccagtgccaccagttcatc

SEQ ID NO: 14 - DNA, 26 bp, Artificial Sequence
  amorce
caagcttcgattccggaagtggtgac

SEQ ID NO: 15 - DNA, 26 bp, Artificial Sequence
  amorce
aggatccatgcccccatgacaaacag

SEQ ID NO: 16 - DNA, 25 bp, Artificial Sequence
  amorce
ggatccaggcgtcggggatatcgtg

SEQ ID NO: 17 - DNA, 31 bp, Artificial Sequence
  amorce
aagcttcggtcttcatacagacccagatagc

SEQ ID NO: 18 - DNA, 26 bp, Artificial Sequence
  amorce
aagcttggccattgccgggggctgtg

SEQ ID NO: 19 - DNA, 27 bp, Artificial Sequence
  amorce
ctcgagccaccgtgtgactgacgaatc

SEQ ID NO: 20 - DNA, 31 bp, Artificial Sequence
  amorce
ttgtcgacttaaggagggcatcatgcctgac

SEQ ID NO: 21 - DNA, 29 bp, Artificial Sequence
  amorce
gttctagatgactgcaagaggtggcttcc

SEQ ID NO: 22 - DNA, 8217 bp, Artificial Sequence
  sequence plasmidique pWKS-GalP
agatctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgctcccggctcgtatgttgtgtggaattgtgagcggataacaatttcacacaggaaacagctatgaccatgattacgccaagcgcgcaattaaccctcactaaagggaacaaaagctgggtaccgggccccccctcgaggtcgacttaaggagggcatcatgcctgacgctaaaaaacaggggcggtcaaacaaggcaatgacgtttttcgtctgcttccttgccgctctggcgggattactctttggcctggatatcggtgtaattgctggcgcactgccgtttattgcagatgaattccagattacttcgcacacgcaagaatgggtcgtaagctccatgatgttcggtgcggcagtcggtgcggtgggcagcggctggctctcctttaaactcgggcgcaaaaagagcctgatgatcggcgcaattttgtttgttgccggttcgctgttctctgcggctgcgccaaacgttgaagtactgattctttcccgcgttctactggggctggcggtgggtgtggcctcttataccgcaccgctgtacctctctgaaattgcgccggaaaaaattcgtggcagtatgatctcgatgtatcagttgatgatcactatcgggatcctcggtgcttatctttctgataccgccttcagctacaccggtgcatggcgctggatgctgggtgtgattatcatcccggcaattttgctgctgattggtgtcttcttcctgccagacagcccacgttggtttgccgccaaacgccgttttgttgatgccgaacgcgtgctgctacgcctgcgtgacaccagcgcggaagcgaaacgcgaactggatgaaatccgtgaaagtttgcaggttaaacagagtggctgggcgctgtttaaagagaacagcaacttccgccgcgcggtgttccttggcgtactgttgcaggtaatgcagcaattcaccgggatgaacgtcatcatgtattacgcgccgaaaatcttcgaactggcgggttataccaacactaccgagcaaatgtgggggaccgtgattgtcggcctgaccaacgtacttgccacctttatcgcaatcggccttgttgaccgctggggacgtaaaccaacgctaacgctgggcttcctggtgatggctgctggcatgggcgtactcggtacaatgatgcatatcggtattcactctccgtcggcgcagtatttcgccatcgccatgctgctgatgtttattgtcggttttgccatgagtgccggtccgctgatttgggtactgtgctccgaaattcagccgctgaaaggccgcgattttggcatcacctgctccactgccaccaactggattgccaacatgatcgttggcgcaacgttcctgaccatgctcaacacgctgggtaacgccaacaccttctgggtgtatgcggctctgaacgtactgtttatcctgctgacattgtggctggtaccggaaaccaaacacgtttcgctggaacatattgaacgtaatctgatgaaaggtcgtaaactgcgcgaaataggcgctcacgattaatctccccaagcttcctcccatcgcggaggaagccacctcttgcagtcatctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaaaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgaaagcaaattcgacccggtcgtcggttcagggcagggtcgttaaatagccgcttatgtctattgctggtttaccggtttattgactaccggaagcagtgtgaccgtgtgcttctcaaatgcctgaggccagtttgctcaggctctccccgtggaggtaataattgacgatatgatcatttattctgcctcccagagcctgataaaaacggttagcgccggggttggatttttcagcgttccagctaaggctaaggcattcctgttcaagcgcaagccgggctataaagcgcattatcgctttacccgtgcccttatttcgatcgcactgagaaacatacagctctttaatatgcaggtaatccgggcagcgcaacggaacattcatcagtgtaaaaatggaatcaataaagccctgcgcagcgcgcagggtcagcctgaatacgcgtttaatgaccagcacagtcgtgatggcaaggtcagaatagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctattctgaccttgccatcacgactgtgctggtcattaaacgcgtattcaggctgaccctgcgcgctgcgcagggctttattgattccatttttacactgatgaatgttccgttgcgctgcccggattacagctgttcgggggttcactgacgctgcgtgactacgatggtcaggttgcggaggctatggccctggtacgagcgctgaacaaaatgacgaaagcaggtatgcctgaaagcgtgcgtattgcctgaaaacacaacccgctacgggggagacttacccgaaatctgatttattcaacaaagccactttcagtatggatttgggtgttacgggggggggttggtgcttaaaatcgcgctgaggatttctgatggtgttaagcgggcggttttgagatgtaaactcgcccgtttaacataatggatcttgcgcgcaccgcccgaacaccactcgccacaaaaaaccgccggaacgtccaaaagtacgggttttgctgcccgcaaacgggctgttctggtgttgctagtttgttatcagaatcgcagatccggcttcagccggtttgccggctgaaagcgctatttcttccagaattgccatgattttttccccacgggaggcgtcactggctcccgtgttgtcggcagctttgattcgataagcagcatcgcctgtttcaggctgtctatgtgtgactgttgagctgtaacaagttgtctcaggtgttcaatttcatgttctagttgctttgttttactggtttcacctgttctattaggtgttacatgctgttcatctgttacattgtcgatctgttcatggtgaacagctttgaatgcaccaaaaactcgtaaaagctctgatgtatctatcttttttacaccgttttcatctgtgcatatggacagttttccctttgatatgtaacggtgaacagttgttctacttttgtttgttagtcttgatgcttcactgatagatacaagagccataagaacctcagatccttccgtatttagccagtatgttctctagtgtggttcgttgtttttgcgtgagccatgagaacgaaccattgagatcatacttactttgcatgtcactcaaaaattttgcctcaaaactggtgagctgaatttttgcagttaaagcatcgtgtagtgtttttcttagtccgttatgtaggtaggaatctgatgtaatggttgttggtattttgtcaccattcatttttatctggttgttctcaagttcggttacgagatccatttgtctatctagttcaacttggaaaatcaacgtatcagtcgggcggcctcgcttatcaaccaccaatttcatattgctgtaagtgtttaaatctttacttattggtttcaaaacccattggttaagccttttaaactcatggtagttattttcaagcattaacatgaacttaaattcatcaaggctaatctctatatttgccttgtgagttttcttttgtgttagttcttttaataaccactcataaatcctcatagagtatttgttttcaaaagacttaacatgttccagattatattttatgaatttttttaactggaaaagataaggcaatatctcttcactaaaaactaattctaatttttcgcttgagaacttggcatagtttgtccactggaaaatctcaaagcctttaaccaaaggattcctgatttccacagttctcgtcatcagctctctggttgctttagctaatacaccataagcattttccctactgatgttcatcatctgagcgtattggttataagtgaacgataccgtccgttctttccttgtagggttttcaatcgtggggttgagtagtgccacacagcataaaattagcttggtttcatgctccgttaagtcatagcgactaatcgctagttcatttgctttgaaaacaactaattcagacatacatctcaattggtctaggtgattttaatcactataccaattgagatgggctagtcaatgataattactagtccttttcctttgagttgtgggtatctgtaaattctgctagacctttgctggaaaacttgtaaattctgctagaccctctgtaaattccgctagacctttgtgtgttttttttgtttatattcaagtggttataatttatagaataaagaaagaataaaaaaagataaaaagaatagatcccagccctgtgtataactcactactttagtcagttccgcagtattacaaaaggatgtcgcaaacgctgtttgctcctctacaaaacagaccttaaaaccctaaaggcttaagtagcaccctcgcaagctcgggcaaatcgctgaatattccttttgtctccgaccatcaggcacctgagtcgctgtctttttcgtgacattcagttcgctgcgctcacggctctggcagtgaatgggggtaaatggcactacaggcgccttttatggattcatgcaaggaaactacccataatacaagaaaagcccgtcacgggcttctcagggcgttttatggcgggtctgcggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccaatgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcg

SEQ ID NO: 23 - DNA, 25 bp, Artificial Sequence
  amorce ptsG FW
cttctctcagtgggctgcttaccag

SEQ ID NO: 24 - DNA, 26 bp, Artificial Sequence
  amorce ptsG RV
ccgttaatcctaatctgccacgcacc

SEQ ID NO: 25 - DNA, 24 bp, Artificial Sequence
  amorce manXYZ FW
tgttaggcgagcaggaaaacgtcg

SEQ ID NO: 26 - DNA, 24 bp, Artificial Sequence
  amorce manXYZ RV
accaatcacacccagagcaaccag

SEQ ID NO: 27 - DNA, 25 bp, Artificial Sequence
  amorce pfkA FW
cattttgcattccaaagttcagagg

SEQ ID NO: 28 - DNA, 26 bp, Artificial Sequence
  amorce pfkA RV
tcatcggtttcagggtaaaggaatct

SEQ ID NO: 29 - DNA, 4903 bp, Artificial Sequence
  Sequence plasmidique pBAD HisA comprenant le gene codant pour la protein Teth514-1788
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgggcattaaactgaaacgcctgagcgataaaccggtgttaatgccgaaagcagaaaacgagtgggaacgcgcagccgtgtttaataccgccgcaatatacgataatggtctgtttcatctgatataccgtgccaccgatattggtcctcacgccaaatatggcaaatatattagtcgcttaggctatgcagtgagtaaagatggtattaattttatgcgcctggataaaccggttatgtctaacgaaaccgaacaggaactgcgcggcttagaagatccgcgcattgtgaaaattgatggcatatattatatgatgtataccggctttggcgatcgctttcaggatgattatcgcatttgtctggccacctctaaaaatctgattgattgggaacgtaaaggcgttgtgctggatgaaccgaacaaggatgcctcactgtttccggaaaaaattaatggcaaatatgtgatgttacatcgtcgctatccggatatttggattgcctttagcgatgatctgaaaaattggtatgatcataaaccgattctgaaaccgattccgaatacctgggaatcagccagagtgggcattggcggcccgccgattaaaaccaaagatggttggtttttaatataccatgcagcagatgataataacgtgtatcgcttaggtgcagtgctgctggatctggaagatccgagcaaagttattgcacgtcagaaagaaccgattctggaaccggaactgggttgggaaaaagaaggctatattccgaatgtggtgttttcttgtggcaatgccgttaaagatgataccatatatgtgtattatggcggtgcagataccgttattggcgttgccattttagagatgaaagatattaaatttcaccatcaccatcaccattaactcgagatctgcagctggtaccatatgggaattcgaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 30 - DNA, 5522 bp, Artificial Sequence
  plasmide pBAD-HisA-Uhgb_MP
agaagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagaaataattttgtttaactttaagaaggagatatacatacccatgggatctgataaaattattcatctgactgatgattcttttgatactgatgtacttaaggcagatggtgcaatcctggttgatttctgggcacactggtgcggtccgtgcaaaatgatcgctccgattctggatgaaatcgctgacgaatatcagggcaaactgaccgttgcaaaactgaacatcgatcacaacccgggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggctctggatccggtgatgacgatgacaagctgggaattatcacaagtttgtacaaaaaagcaggctccgcggccgcccccttcaccatgagtatgagtagcaaagttattattccttgggaagaacgtccggcaggatgcaaggacgtattatggcgcagtgtggcaaaccccattattccgcgtgatttgcttcccacttccaacagtattttcaacagtgcggtagtacctttcggcgacgggtttgcaggagtattccgctgcgacgatactagccgtcgcatgcggctgcacgtagggttcagtaaagatgccataaactggaacatcaaggaagaaccgttgaagttccaatgcgatgatgaggaaatcggtacatgggtgtatggctacgacccgcgcgtgtgctttatcgaagaccgctattatgttacctggtgcaacggctatcacggtcctactatcggtgtggcctatacgttcgatttcgagacgttccaccagttggaaaacgcttttattcctttcaaccgtaacggagtacttttcccacgcaagataaacggtcgttttgccatgttgagccgtccgagcgacaatgggcatactccctttggagatattttctacagtgagtcgcccgatatggagttttggggacgccaccgccacgtgatgtcacctgccgcttttgaggtaagtgcatggcaatgcaccaaaatcggtgcaggtcctattcctgtcgagactcccgaaggctggctgctgatttatcacggtgtgctgcattcctgcaacggttatgtttacagtttcggctctgctctgctcgatttggacgaaccgtggaaggtaaagttccgctcagggccttatctgcttgccccgcgcgaaccgtacgaatgtatgggcgatgttcctaatgtatgtttcccgtgcgccgctttgcacgacaatgaaacgggcaggattgccatatactatggctgtgccgataccgtaaccggactggcattcggctatattcccgaaattatagaatttaccaaacgtacaagcatcatcaagggtgggcgcgccgacccagctttcttgtacaaagtggtgatcaagcttgaaggtaagcctatccctaaccctctcctcggtctcgattctacgcgtaccggtcatcatcaccatcaccattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcag

SEQ ID NO: 31 - DNA, 27 bp, Artificial Sequence
  amorce fwd
gaggaaggatccatgggcattaaactg

SEQ ID NO: 32 - DNA, 24 bp, Artificial Sequence
  amorce rev
gctgcagatgaattcttaatggtg

SEQ ID NO: 33 - DNA, 28 bp, Artificial Sequence
  amorce Uhgb fw
caccatgagtatgagtagcaaagttatt

SEQ ID NO: 34 - DNA, 27 bp, Artificial Sequence
  amorce Uhgb rev
gatgatgcttgtacgtttggtaaattc

SEQ ID NO: 35 - DNA, 35 bp, Artificial Sequence
  amorce Fw
gaggaattaaccatgctgagcgtggaaaaacgctg

SEQ ID NO: 36 - DNA, 41 bp, Artificial Sequence
  amorce rev
agctgcagatctcgatcagtggtggtggtggtggtgctcga

SEQ ID NO: 37 - DNA, 5175 bp, Artificial Sequence
  pBAD- beta-1,3-mannooligosaccharide phosphorylase
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgctgagcgtggaaaaacgctgtattctgagtccggtgccgggtagcccgtgggctggtaaaatggttctgaatccggcaatgattgcagatccggaagatccggaaaaaattcacatgctgtttcgtgccaccggcccgtggccgcaggctcgtttaccggaacgtccgctgccgtatccgatttttctgggttatggtgtgagttgcaatggcggtgcagattggcagtttgatctgagtcgtccggcaatggcaccgcgcctggaatttgatcgcgatcgctttctggcacgcggcatgcgtgatggcgtgatgtttgattatgcaaatggttgtattgaggatccgcgtctgttttattttgaagaacagctgtatctgagtgtggcatgtcgcgcatttccgccgggtccgtattgggaaaaagatgatccggttcagtgcatgccggaatgggcattcaaactgaatccggccgttcgcgaaaatagcaccgttaccatgctgtatcgcgttgatctggccgcactggcagccggccgctatgctgaagcctttagcccggttggtccgctgcatgatccggatctgagtgatgatcgtgatgcaatgctgtttccgcgtcgcctggaaattggcggtaaacgcaaaattgcctgcctgcatcgtccgaaacagccgtggaattatgcctgcggtaaagaagccgccgcaccgagcatttttctggcctttggcgatgaactgagcgattttcataccggtaaagcccgtacccagctgtttgccggtccggcctttgcccatgaagccaatcgcgtgggtgcaagttgggcaccgctggaactggaaccgggcctgtggctgctgccgtatcatggcaaacaggatgatcgtgttggttatacccagaattttatgctgctgcgcgaaaatggccgccaggccccggatattattgcacgcccggccgaacgcctgctgtatgccaccgaaccgtgggaactggaaggcgaattcactattccgtgtctgtttacctgtagtggtctgctgcgcccggatggcaccctgctgatgggttatggcgccgccgatcgtaaagttggcctggccagcattcgtctggaagaactgctggccgtgctgcgtccgctcgagcaccaccaccaccaccactgatcgagatctgcagctggtaccatatgggaattcgaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 38 - DNA, 6505 bp, Artificial Sequence
  pBAD-ACL0729 6505 bp
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatgggccaccaccaccaccatcacaaaaaaaaacaaagcatcaaaaagaactacggccaatacctgccactgtacaacctgggccaattaaaaagcagcatcacccccttcttcggcggcgatattaaaaccgaccaccaccactacctgttagaaccgaccaccgaaaacgatttatatgccaaccatatgagccgtaatattatttttaccatcgacgacgaaatgtactttctgaatggtaacacctatctgcagcagaatgatgagctgcaagttgaatatgacctgctgtatcagcgcgttgttagaagcaataccctgtttagcatcgaaagcctgtcctttattcccctgcatgacaacctggaattattagaagtgaccatcaaaaatacctccaataaaaatctgacccttgatgttattaccgcaattcctatttatgcccgttcagcagataatattcgtgaccaccgtcatgttaccagcctgctgaataaagttgaaatcaatgaacagaccgtgctggttaaaccgaccctgagctttgacgaacgtgggcatcatctgaatcatgaagcgtataccgtttctgcatatagcccgaccaccaaagtaaatcgcatcatcaccagcttagaagattttattgacggcggtagctttaattttcctaaaggtatttaccgtaaggattttgaaccaaagcctggatatgaggttatcggcgcaattggttttgaaccgtttattctgaaaccggaggagaccgcaacatttaatattagcatgggcatttttaaccagaatgaagatgcactgcacattattaacaaatataagacaaagaagagcgtggaagaagcgtttaaacacatgagaaattatttcgaagccgaaagcagcaaattaagcattagcatggttaataaggacgttgaactgctgctgcggtgggtgcgctttcagccggttctgcgtcgtatctttggtaacagctacctgccgcatcatgattatggccgtggcggtcgtggatggcgggacttatggcaggatttattaagcttaattctgattaacgacgagagtgttgaaacactgttatttaataacttccagggtattcgtatcgacggtagcaatgcaacaattattggtgataagccgggtgagtttctggcagatcgcaataaaatcgttagagtttggagcgaccacggtgcctggccgctgttaacaaccctgagctatattcatagcacgggacacattgaatttctgtttaaacagcagacctattttcatgaccaatttacccactataccaataaaaccagagatattgaaaccagcgaacatatattacgtgtgcacaacaaaccgtacttagggaccattctggaacatttactgctgcagaacattgttgcacactttaataccggcaaatttggctttgtgcggctggaaaatgcagattggaatgatggtctggacatggccaaaaataatggtgaaacaattgcatttacccatttctacgcaagcaatctgatgaaaatggcagaggtgatcgaaaaaatcaaccgtccggtgcgtctgtttaaaagcctgtacgatctgatctttgaaacaatcgacctgaagaccttttttgataatgttagcgatttcaaggacaccaccgttgaagtggacgcaaccaaattaagctatcaactggtagaactgtataaaaaacgcattgatcacattaacgagaatgcatggattaaagacacccacctgcagagctatatcaccgatagcaatgtttttctggacaccgcagataccatgaatctgaccggtcaagcaatggcactgttaaacgaaaccctgaccatgagccaggcaaaaagcgtcgcagataccaccaaaaaactgctgtttaataaagataacggcggttaccacctgaatagcaattaccataaaataatgatggacatgggccgtgcatttggatttagttacaaccataaagaaaacggcgcagtgttttctcatatggcaataatgtatgtttacggcctttataattacaaccttgttcagctgggtaatgagggttataaaagcttaattatgcgtgccattcatccggacgcacatacggttctgggtattccggaatactttaccgataaaggtgtcggtaagtatttatatcttactggtagcgcaagttggctgctgaaactgctgcaagagcaggtttttggtattacattttattacgggaagctgacgctgaatccgaagctggttcgtgaagattttatcaatcatcgcgcgagcattcagacgtttattcatggtaaattaatcaagatcacctacattaacctgaaaaatctggactatggcgcatacaaaatcgataaggttctgtatcagggtcaaccgattaatatagaacatacaaagatcgaaggggatattgaagtgtacctggatgaactgtaactcgagatctgcagctggtaccatatgggaattcgaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 39 - DNA, 70 bp, Artificial Sequence
  amorce FW
atgagcacagaaaaagaaaagatgattgctggtgagttgtatcgctcggcgtgtaggctggagctgcttc

SEQ ID NO: 40 - DNA, 70 bp, Artificial Sequence
  amorce RV
ttacaattttttaattattctggctggattaccgcccacgacaacgttgtcatatgaatatcctccttag

SEQ ID NO: 41 - DNA, 24 bp, Artificial Sequence
  amorce fw
gatgattgctggtgagttgtatcg

SEQ ID NO: 42 - DNA, 24 bp, Artificial Sequence
  amorce rv
ttaattattctggctggattaccg

SEQ ID NO: 43 - DNA, 70 bp, Artificial Sequence
  amorce FW
attatgcgcagcacagccactctccattcaggttcatccaaacaaacacagtgtaggctggagctgcttc

SEQ ID NO: 44 - DNA, 70 bp, Artificial Sequence
  amorce RV
acacgcgctaaacggccgtggcctttgacagtcaccggtgattcgttggccatatgaatatcctccttag

SEQ ID NO: 45 - DNA, 23 bp, Artificial Sequence
  amorce FW
gtgcgggtctgacgcctaaatac

SEQ ID NO: 46 - DNA, 23 bp, Artificial Sequence
  amorce rv
gatgggtttcaatcttcttgcct

SEQ ID NO: 47 - DNA, 40 bp, Artificial Sequence
  amorce FW
aaataggcgctcacgattaaaaggaagctgagttggctgc

SEQ ID NO: 48 - DNA, 40 bp, Artificial Sequence
  amorce RV
tagctttgcactgtttcagagtgaagacgaaagggcctcg

SEQ ID NO: 49 - DNA, 40 bp, Artificial Sequence
  amorce FW
cgaggccctttcgtcttcactctgaaacagtgcaaagcta

SEQ ID NO: 50 - DNA, 40 bp, Artificial Sequence
  amorce RV
tgttttttagcgtcaggcatctctaggattcctccggttc

SEQ ID NO: 51 - DNA, 40 bp, Artificial Sequence
  amorce FW
gaaccggaggaatcctagagatgcctgacgctaaaaaaca

SEQ ID NO: 52 - DNA, 40 bp, Artificial Sequence
  amorce RV
gcagccaactcagcttccttttaatcgtgagcgcctattt

SEQ ID NO: 53 - DNA, 20 bp, Artificial Sequence
  amorce FW
tctgaaacagtgcaaagcta

SEQ ID NO: 54 - DNA, 20 bp, Artificial Sequence
  amorce RV
ttaatcgtgagcgcctattt

SEQ ID NO: 55 - DNA, 5080 bp, Artificial Sequence
  sequence plasmidique du vecteur pBAD- Lin0857
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgttttttgggctaacaggaggaattaaccatggggaatatctatcgctacgaagaaaatccgcttattactccgttggatgtgaagcctattcatgagggatttgaagtgatcggcgcctttaacggcggtgtcgcggaatacaatggtgaagttctgctgctgctgcgcgtcgccgaaaaaccggttagcgaagatcccgaaatcgttctggcgccagtgtataatgctaagaataaagaattagaactgcaaagtttccgcctggacgatgaaaactacgacttcgaagatccgcgtatgattcgtagcaaagcgaaattggaaggatttagctatttgacctcgctcagttatatccgcatcgcgcgctcaaaagacggccaccacttcacactggatgaaaagccattcctgtatccattcaatgaatatcagaccttcggtattgaggacgctcgcgttacccaaattggagatacgtaccatgtgaacttttctgcggtctcagagtttggcgtcgctgatgccttggtgacgactaaggactttgaaaatctggaataccagggcaacatctttgcaccagaaaacaaagatgtactcatcttcccagaaaaaatcaatggtaagtattacgcgcttcaccggcccagcctcaaaagtattggcaatctggacatctggatcgcaagctcaccggacctgcggtccttcggcgatcatcgccatctcctcggtatccgtccgggcgaatatgattctggccgggtgggtggcggatgtgtgccgattaaaacagaagaaggctggttgattttgtaccacggagccaccgaagaaaatcgttatgtgatgggtgccgctttgctggatttgaatgatccaacaatcgtgttaaagcgcacgaaaactccgattctggagccggtagctgattacgagaaaaatggcttttttggcgatgtcgtttttgcctgtggtgcgatccaggaaggtgataccttacatatgtactacggcgtcgcggatacttcgatggctggctgcgatatgaagattagcgagattctccatcagttagaggttgaggcgaaacaccatcaccatcaccattaactcgagatctgcagctggtaccatatgggaattcgaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 56 - DNA, 72 bp, Artificial Sequence
  amorce fw
gcacaataacatcattcttcctgatcacgtttcaccgcagattatcatcaagattgcagcattacacgtctt

SEQ ID NO: 57 - DNA, 77 bp, Artificial Sequence
  amorce rev
tgtgggtttccgcgaactgctgccacgggttagctttgcactgtttcagatccatatgaatatcctccttagttcct

SEQ ID NO: 58 - DNA, 77 bp, Artificial Sequence
  amorce fw
aggaactaaggaggatattcatatggatctgaaacagtgcaaagctaacccgtggcagcagttcgcggaaacccaca

SEQ ID NO: 59 - DNA, 72 bp, Artificial Sequence
  amorce rev
aaaaacgtcattgccttgtttgaccgcccctgttttttagcgtcaggcatctctaggattcctccggttcct

SEQ ID NO: 60 - DNA, 29 bp, Artificial Sequence
  amorce fw
gcacaataacatcattcttcctgatcacg

SEQ ID NO: 61 - DNA, 26 bp, Artificial Sequence
  amorce rev
aaaaacgtcattgccttgtttgaccg

SEQ ID NO: 62 - DNA, 24 bp, Artificial Sequence
  amorce fw
cagcctgtctgttcgtgcgaaaga

SEQ ID NO: 63 - DNA, 19 bp, Artificial Sequence
  amorce rev
tcagagaggtacagcggtg

SEQ ID NO: 64 - DNA, 20 bp, Artificial Sequence
  amorce pBAB-fw
atgccatagcatttttatcc

SEQ ID NO: 65 - DNA, 18 bp, Artificial Sequence
  amorce pBAD-rev
gatttaatctgtatcagg

SEQ ID NO: 66 - DNA, 799 bp, Artificial Sequence
  promoteur IHF
tctgaaacagtgcaaagctaacccgtggcagcagttcgcggaaacccacaacaagggcgaccgtgttgaaggtaaaatcaagtctatcactgacttcggtatcttcatcggcttggacggcggcatcgacggcctggttcacctgtctgacatctcctggaacgttgcaggcgaagaagcagttcgtgaatacaaaaaaggcgacgaaatcgctgcagttgttctgcaggttgacgcagaacgtgaacgtatctccctgggcgttaaacagctcgcagaagatccgttcaacaactgggttgctctgaacaagaaaggcgctatcgtaaccggtaaagtaactgcagttgacgctaaaggcgcaaccgtagaactggctgacggcgttgaaggttacctgcgtgcttctgaagcatcccgtgaccgcgttgaagacgctaccctggttctgagcgttggcgacgaagttgaagctaaattcaccggcgttgatcgtaaaaaccgcgcaatcagcctgtctgttcgtgcgaaagacgaagctgacgagaaagatgcaatcgcaactgttaacaaacaggaagatgcaaacttctccaacaacgcaatggctgaagctttcaaagcagctaaaggcgagtaattctctgactcttcgggatttttattccgaagtttgttgagtttacttgacagattgcaggtttcgtcctgtaatcaagcactaagggcggctacggccgcccttaatcaatgcagcaacagcagccgcttaatttgcctttaaggaaccggaggaatc